SEQUENCE LISTING (152 sequences)

SEQ ID NO: 1 - DNA, 4378 bp, Oryza longistaminata
tgccaatatctcttgcctcttggaagctgggactaattgggagctggaataatagatttcaagaccttattttaaacgtgcagtgatagtattactcctattctcctactgtccaagcccacaaaggtcatcacctgcaccgttgcctgactatttatacaggagtaacaggaccccaagatgacagcatgctactgctccatctagagcgtcgttgttcaccagcagcaaccatcttttcacgctatggcgtagccagagtacacatctagtgctcctatatgcctcttggtctttgactcgcctccttcttgcctctctggcctgcagaaccatgcatggaccgaaaggagaacagatccattccttctgtaccatatgccccatcagcaatccctcctgccatcattcttccttatggtctaatttcggtgagctgttttaatcaaagacacccaacattatggcattattctccaaccttgtcaaaataattgcatacagtaaaaacagatggacactgtcatgctaagtaagagtactaacagtagtactccagcatacactatggcacctggcagatcagttgcactacctgaattaagtgctggattaggatacatacccccaatatcttcaacttaacattacacacacacattggcattcatggatggtctaaaaattgagtcccatggctgcataatggttgctactgtcggttggatctgatatgatttctctctttgctgtctcctcagtcttccaggaatggcaggctagtcaaagctcccgaccccatgacccaggtggcaatcggagcaaggctaaatagtatagccgacctgttgactatgaagtaagtttttttagtcttctctcattccacttattagagcgagccaatctaatagttcatttatacaatagttaactataaaaatatactacactattaatactcggtcccacctctcatacacacataatatcttggagcccgtgttgcagccggctacaaatctgtagcccgctttctcctctctcttctcttttcttctcgatatatggttatagccggcttatagcatgctattgtacctgctcttatatagttactatctctttacaattaatatagggtctacttgtctctctcacagaattttttggttcttgtgtcccctgcttctcctctcttctctcctccacctcagcattcagccggcttgtaccctactgccactgtacttgctctcaatggctggcccggtcggtaaaacctcaccgttttaccgtggttggtcgcatcttgtccagatatatagccagaaatttgttcttttttgatcggatggagtactattatacttgctctcaatggctggcccggtcggtaaacctcaccgttttaccgtggttgctcgcacccgtacgtacactactgttgcccagcctgcacctgcactgcagcagctagacaggcacgagtcagagcagagatctgatctcacctcactcgcctcgatcacattgcctgtctagaagagatgagatgagagcagcaggactgaactctcaccattttaaaatataagaattttaagattattttttaagaaaagtactgtactacaagatactacgtataagaaaaaacattctcctgtgatcagctaaaatttaaatttctttccctaaaaaaagataaattttaaatttcttagatttttttatcaggcatcacatttaaactctcaccgttttaatatatgactttttagattattttagtaaaatacaaagatatataagaaaaaaaattatcttttagtcactttaggagctgagctaaattttaagttgcttagactttcttgtctgacatctcattggttagaaatatattttcctcccaatggatgagctagaaagtgccccatcggaagctagatagaacactttgtgacagaggaagtagtacaaggctatgtttagttccatccaaagtttggattttggttgaaattgagaatgatgtgactgaaaagttgtgtgtgtatgataggttgatgtgatgggaaaatgcaaagtttggaggcaaactttggatctaaacacaccctaaattttcatgcattgaaatcaataaatatttatattagagtaaaattttaaatcatagaaatgcttccatttgagaacgacgtagtagcatagcagacttttaaagccctaacttcataggattcacgacctacatggaaatttttctaccctttgttacaaagaattggaactttttaaatcccataaaattcttatggaatggtaattgacgtataggttttgaagaaaacttagcaagaatttcaacttcttgaaaaattttctttaagtcgtctctctcattcaactcgtatattttttctgtgatctaatcaaacccaaaaaaacttagcaagccctttcatatgttttatgtgtcttttaattctatattttacacttcaaatttatctttttatttctacgtttagggtgggaaattaaagcgagagattaggctgaaagatctagaaatcctaggtccaaaaaagacagggtcgtgacggtgccgagagccgtcctgccgccctggaaattgtgcctatgtaaagtgtagccggccacacgttgcttcttcctttccttcacatctcacagcccccagtccagctcgctcgccgtctcgccattttgaccagaccacctcgctgcctctgctctgctcagtgctgctcactgcctcactcacactgtcacagtgctcttctgggtataagtagctgggcgcgcggcgtcaccttccttggctgcccgtgagcttcccgcgcgcgccatggcacgggctcgtggccgtgggcgagcttcttggtaggcgaggcgtcggcggcgatcgagttgtctcgagagctactcagctatgaggagagcggccacggcgcctctcgccgccgccgccgcggtgttcctctctgcgttgctgctcgcctccgcctccgcctccgccttcaggctccctcctcctcgccgtcttcttcccctggtacgcgtacgcctcgccgccgccgttcttgatcgacctgagattttccttttttgttcggttggttgatgtgctgtgtgcgtccatggcgatgatgcaggttggtggcgaggtggcggtggcggtggcagctggggaggaggagaaggtgcggctggggtcgagcccgccgagctgctacagcaagtgctacgggtgcagcccgtgcgtcgcggtgcaggtgcccaccctgtccgccccgtccgtccccgccgccgccgccgccgcgcacgacgccgcgccgctcgtggccacgttcaccaactacaagccgctcgggtggaagtgccagtgccgcgaccgcctgttcgacccctgaggcgcgcgcgcgctctcgctgtgaattacggtgtgtgtggccgcgtcggcccgtgcccggcagcacatggcgctgcactgctgctgatgctggtggtggtggaatcgttgtcgtcgcgtcgggttgagagggattgttgatagattccgtgtaatatgccaggacaaaattttgtcaccgctgctgctgcccatgcagctggatcggctcggctactctcccacctacatatactgtatctttcactggcatctgctcgccgttttggaatctctgcggtggtggggttgcatcttgcatgtacacatgtttttcatggatcgatccatgcctactccatggaatctaatgggaatccatcatcattcacgctggatggatggatggatgtagtgaatggtagtttttcttatttttgttggagatggatattttttactttacgtctaatcagatatatggtgtcttttaaattgagaatttagctactcaaacaatccattctgaaattcgttcaaatgaagaattgaacttaggatcttaggctgcgttcggtagaccaggttctcaactcttcctcattttccacgcgcacgtttttcaaacagctaaacggtgcgtttttacaaaaagtttctatacgaaagttgcttaaaaaatcaaattaatctatttttttaaaaaatatctaatatttaattaaatcacacgctaattgctgctccgttttacatgccagggaatgagggttcgagtgttattcagatcgctgcaatctacctgccctttcgcaaagtgaacggtatagttctagtggtaaattggtaattgatccctctctccatatgcatgcatgcatttgcttgatagtatgtttttgtgcgttgatgtgcataggtcagttttgttcatttgaggtttcatgccaggga

SEQ ID NO: 2 - DNA, 4397 bp, Artificial sequence
  OLLS1 nucleic acid sequence from NIL_107B-12
tgccaatatctcttgcctcttggaagttgggactaattgggagctggaagaatagatttcaagaccttattttaaacgtgcagtgatagtattactcctattctcctactgtccaaacccacgaaggtcatcacctgcaccgttgcctgactatttatacaggagtaacaggaccccaagatgacagcatgctactgctccatctagagcgtcgttgttcaccagcagcaaccatcttttcacgctatggcgtagccagagtacacatctagtgctcctatatgcctcttggtctttgactcgcctccttcttgcctctctggcctgcagaaccatgcatggaccgaaaggagaacagatccattccttctgtaccatactgccccatcagcaatccctcctgccatcattcttccttgtggtctgatttcggtgagctgttttaatcaaagacacccaatattatggcattattctccaaccttgtcaaaataattgcatacagtaaaaacagatggacactgtcatgctaagtaagagtactaacagtagtactccagcatactccgtactacactatatggcacctgacagatcagttgcaccacctgaattaagtgctggttttggatacatacccccaatatcaacttaacattacacacacacattggcattcatgcatggtctaaaaaattgagtcccatagctgcataagagcaggtacaatagcaggctataagccagctacaaacatattttaagaagataaattaggagagagaagagcagcgggctacagatttgtagccagctgtagcacggacttcaagacacagtgtgtctatgacaggtgggaccaggtattaatagtgtattcagtatgtaactattgtatgaataagctattagattggttatagatgaattgaaactagtactccagttggctatactattgaacttgctctaatggttgctactgtcggttggatctgatatgatttctctctttgctgtctcctcagtcttccaggaatggcaggctagtcaaagctcccgaccccatgacccaggtggcaatcaatggctggcccggttggtaaaacctcaccgtttatttaccgtggttactcgcacccgtacgtacactactgttgcccagcctgcacctgcactgcagcagctagacaggcacgagtcagagcagagatctgatctcacctcactcgcctcgatcacattgcctgtctagaagagatgagatgagagcagcagaactctcaccattttaaaatataagaattttaagattattttttaagaaaagtactgtactacaagatactaatccctccgtccaaaaaaaaaaaaaagcaaactctagatttccgtgtccaattttaactatctgtcttatatgaaatttttttataattcttattttcattgttattagatgataaaacatgattaatattttatgtgtgacttgtctttttaatttttttcataattttttcaaataagacgaacggtcaaacattgggcacggaaatcagggtttgtcttttttttttgggacggagggagtacgtataagaaaaaacattctcttgtgatcagctaaattttaaatttctttccctaaaaaaagataaattttaaatttcttagatttttttatcaggcatcacatttaaactctcaccgttttaatatatgactttttagattattttagtaatatacaaagatatatagaaaaaaaattatcttttagtcactttaggagcttagctaaattttaagttgcttagactttcttgtctgacatctcagggtgtgtttagttgggaaaaggaaattttttagtttcacatcgaacgtttgaccggatgtcggaaggggttttcggatacgaatgaaaaaactaatttcagaactcgcctggaaaccgtgaaacgaatcttttgagactaattaagccgtcattagcacatgtgggttactgtagcacttgtggctaatcacggactaattaggcttaaaagattcgtctcacgatttcccccataactgtgcaattagttttttttatctatatttaatgcttcatgcatatgtccaaagattcgatgtgatgtttttgggaaaaaaatttggagaactaatcgggccctcattggttagaaatatattttcctcccaatggatgaggtagaaagtgccccatcggaagctagatagaacactttgtgacagaggaagtagtataaattttcatgcattgaaatcaataaatatttatattagagtaaaattttaaatcatagaaatgcttccatttgagaacgacgtagtagcatagcagacttttaaagccctttcgaatcgagttataaagaatttcataggattcacgacctacatggaaatttttctaccctttgttacaaagaattggaactttttaaatcccataaaattcttatggaatgataattgacgtataggttttgaagaaaacttagcaagaattttaacttcttgaaaatttttctgagtcgtctctctcattcaactcgtacattttttctgtgatctaatcaaacccaaaaaaaaaacttagcaagccctttcatatgttttatgtgtcttttaattctatattttacacttcaaatttatctttttatttctacgtttagggtgggaaattaaagcgagagattaggctgaaagatctagaaatcctaggtccaaaagaagacagggtcgtgacggtgccgagagccgtcctgccgccctggaaattgtgcctatgtaaagtgtagccggccacacgttgtttcttcctttccttcacatctcacagccccgtctcgctgcttccagtccagcccgctcgccgtctcgccattttgaccagaccacctcgctgcctctgctctgctctgctcagtgctgctcactgcctcactcacactgtcacagtgctcttctgggtataagtagctgggcgcgcggcgtcgccttccttggctgcccgtgagcttccctcgcgcgccatggcacgggctcgtggccgtgggcgagcttcttggtaggcgtcggcggcgatcgagttgtctcgagagctactcagctatgaggacggcggccacgctgcctctcgccgccgtcgccgcggtgttcctctctgcgttgctgctcgcctccgcctccgcctccaggctccctcctcctcgccgtcttcttcccctggtacgcgtacgccctcgccgccgccgttcttgatctacctgagttttttttttaaaaaaaaatgttcggttagttgatgcgctgtgtgtgcgtccatggcgatgcaggttggtggcgaggtggtggtggctggggaggaggagaaggtgcggctaggctcgagcccgccgagctgctacagcaagtgctacgggtgcagcccgtgcatcgcggtgcaggtgcccaccctgtccgccccgtccgtccccgccgccgccgccgccgcgcacgacgccgcgccgctcgtggccacgttcaccaactacaagccgctcgggtggaagtgccagtgccgcgaccgcctgttcgacccctgaggcgcgcgcgcgccccgtggcgcggcgcgctcgcgctctcgctgtgaattacggtgtgtgtggccgcgtcgcgcgcgcgcggcgcgcgcggcccgtgcccggcagcacatggcgctgcactgctgctgatgctggtggtggtggaatcgttgtcgtcgcgtcgggttgagagggattgttgatagattccgtgtaatatgccaggacaaaattttgtcaccgctgctgctgcccatgcagctggatcggctcggctactctcccacctatactgtgtatctttcactggcatctgctcgccgttttggaatctctgcggtggtggggttgcatcttgcatgtacacatgtttttcatgtatcgatccatgcctcctccatggaatctaatggaaatccatcatcattcatgctggatggatggatggatgtagtgagtggtagtttttcttatttttgttggagatggatattttttacttggcctttacatctaaccggatatttgccgtcttttaaattgagaatttagctactcaaacaatacattctgaaattcgttcaaatgaagatttgaacttagaatcttagagtgttattcagatcgctgcaatctacatgccctttcgcaaagtgaacggtatatttctagtggtaaattggtaattggtccctctctgcatctgctatgcacgcatgcatttgcttgatagtatgtttttgtgcgttgatgtgcataagtcagttttgttcatttgaggtttcatgccaggga

SEQ ID NO: 3 - DNA, 4373 bp, Oryza glumaepatula
tgccaatatctcttgcctcttggaagtgggactaattgggagctggaagaatagatttcaagaccttattttaagggtatgtttggttgttgcctaacattgctataactcaatttgccataggtgtggcatggcaaacaaagtgtggccttgttggccacaagtgtgacaatgtttggctagaaaatgagtatatgacatgtagacccatgaaataagaaagtgtggcttgccacaactgaaacaagcaaccaaacgctaagctaacaatctgtggcatgcctaacatgtggtgtggcaatgtgtggcaccaaaccaaacaacccaaacgtgcagtgataatactcctattctcctactgtccaaacccacaaaggtcatcacctgcaccgttgcctgactatttatacaggagtaacaggaccccaagatgacagcatgctactgctccatctagagcgtcgttgttcaccagcagcaaccatcttttcacgctatggcgtagccagagacccagagtacacatctagtgctcctatatgcctcttggtctttgactcgcctccttcttgcctctctggcctgccgaaccatgcatggaccgaaaggagaacagatccattccttctgtaccatactgccccatcagcaatccctcctgccatcattcttccttatggtctgatttcggtgagctgttttaatcaaagacacccaatattatggcattattctccaaccttgtcaaaataattgcatacagtaagaacagatggacactgtcatgctaagtaagagtactaactgtagtactccagcatacactatggcacctggcagatcagttgcaccacctgaattaaatgctggatttggatacatacccccaatatcttcaacttaacattacacacacacattggcattcatgcattgtctaaaaattgagtcccatggctgctaataatggttgctactgtcggttggatctgatatgatttctctctttgctgtctcctcagtcttccaggaatggcaggctagtcaaagctcccgaccccatgacccaggtggcaatcaatggctggcccggttggtaaaacctcaccgttttatttaccgtggttactcgcacccgtacgtacactactgttgcccagcctgcacctgcgctgcagcagctagacaggcacgagtcagagcagagatctgatctcacctcactcgcctcgatcactttgcctgtgtagaagagatgagatgagagcagcaggactgaactctcgccatttcaaaatataaaaattttaagattgttttttaagtaaagtattgtataagatataagaaaaaacactcttgtgatcactttaggagctaaattttaaatcttatttttttttatcaggcatcacatttaaactctcaccattttaatatatgactttttagattattttagtaaaatacaaagatatataagaaaaaaaaattctcttttagtcactttaggagctgagctaaattttaagttgcttagactttcttgtctgacatctcattggttagaaatatattttcctcccaatggataagctagaaagtgtcccatcggaagctaggtagaacactttgtgacagaggaagtagtataaattttcatgcattgaaatcaataaatatttatattagagtaaaattttaaatcatagaaatgcttccatttgagaacgacgtaggagcatagcagacttttaaagccctttcgaatcgagttataaagaatttcataggattcacgacctacatggaaatttttctaccctttgttacaaagaattggaactttttaaatcccataaaatttttatggaatggtaattgacgtataggttttgaagaaaacttagcaaaaatttcaacttcttgaaaatttttctttgagtcgtctctctcattcaactcgtacattttttctgtgatctaatcaaaccgaaaaaaacttagcaagccctttcatatgttttatgtgtcttttaattctctattttacacttcaaatttatctttttatttctacgtttagggcccctttgaatcgcagggttgaaaaaaatagaggaataggaaaaacataggattctgataggaatcgaagtgtaaaacagaggattgcaaaacacaggaatggtcgtttgattggagcgcaggaaaaacgcaggaatcgaatgagagagatagattcaaaggaaaattttcaagaggttagagctcttgctaaattttctccaaaatccacatgctatgtgtcatttcataggaatttcataggatttgaaaaacttcaattctttgaatcaaagggccaaataaaaaaaatttctatagaatttaaatcctataaaatttctacataaatcctgaaaaattaaagcgagagattaggctgaaagatctagagatcctaggtccaaaagaagacagggtcgtgacggtgccgagagccgtcctgccgccctggaatttgtgcctatgtaaagtgtagccggccacacgttgtttcttcctttccttcacatctcacagccccgtcttgctgcttccagtccagcccgctcgccgtctcgccattttgaccagaccacctcgctgcctctgctctgctcagtgctgctcactgcctcactcacactgtcacagtgctcttctgggtataagtagctgggcgcgcggcgtcaccttccttggctgcccgtgagcttcccgcgcgcgccatggcacggcctcgtgggcgtgggcgagcttcttggtaggcgaggtgtcgccggcgatcgagttgtctcgagagctactcagctatgaggacggcgggcacgccgcctctcgccgccgccgccgccgccgtcgcggcagtgttcctctctgcgttgctgctcgcctccgcctccgcctccaggctccctcctcctcgccgtcttcttcccctggtacgcgtacgccctcgccgccgccgttcttgatcgacctgagtgttttttaaaaattttgttcggttagttgatgcgctgtgtgttcgtccatggcgatgccgatgcaggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgcgcacgacgccgcgccgctcgtggcgacgttcaccaactacaagccgctagggtggaagtgccagtgccgcgaccgcctgttcgacccctgaccctgaggcgcgcgcgccccgtggcgcgccgtggcgtggcgtggcgcgtgcatggcggggctcgcgctcgcgctctcgctgtgaattacggtgtgtgtggccgcgtcgcgcgcgcgcgcgcggcccgtgcccggcagcacatggcgctgcactgctgctgatgctggtggtggtggaatcgttgtcgtcgcgtcgggctgagagggattgttgatagattccgtgtaatatgccaggacaaaattttgtcaccgctgctgctgcccatgcagctggatcggctcggctactctcacccacctatactgtatctttcactggcatctgctcgccgttttggaatctctgcggtgtggggttgcctcttgcatgtacacatgtttttcatgtatcgatccatgcctcctccatggaatctaatgggaatccatcatcgttcatgctggatggatggatggatgtagtgaatggtagtttttcttatttttgttggagatggatattttttactttacatctaatcggatatatgttgtcttttaaattgagaatttagctactcaaacaatccattctgaaattcgttcaaacaaagatttgaatttagtatcttaggctgcgttcggcagaccaggttcccaactcctccttattttccgcgcgcacgcttttcaaactactaaacggtgctttttgcaaaaagtttctatacgaaagttgcttaaaaaatcaaattaatctatttttgaaaaaaaatagctaatatttaattaaatcacgcgctaatcactattccgttttgcgtgccggggagtgagggttccccgaacacagccttagagtgttattcagatcgctgcaatctacatgccctttcgcaaagtgaacggtatagttctagtggtaaattggtaattggtccctctctgcatctgctatgcatgcatgcatttgcttgatagtatgtttttgtgcgttgatgtgcataggtcagttttgttcatttgaggtttcatgccaggga

SEQ ID NO: 4 - DNA, 4382 bp, Oryza barthii
tgccaatacctcttgcctcttggaagttgggactaattgggagctggaagaatagatttcaagaccttattttaagggtatgtttggttgttgcctaacattgccataactcaatttgccataggtgtggcatggcaaacaaagtgtgttgttggccacaagtgtggcaatgtttggctagaaaatgagtatatgacatgtaggcccatgaaacaagaaagtgtggcttgccacaactgaaacaagcaaccaaacgctaagctaacaatctgtggcatgcttatcatgtggtgtggcaatgtgtggcaccaaaccaaacaaccccaaaacgtgcagtgatagtactcctattctcctactgtccaaacccacaaaggtcatcacctgcaccgttgcctgactatttatacaggagtaacaggaccccaagatgacagcatgctactgctccatctagagcgtcgttgttcaccagcagcaaccatcttttcacgctatggcgtagccagagacccagagtacacatctagtgctcctatatgcctcttggtctttgactcgcctccttcttgcctctctggcctgcagaaccatgcatggaccgaaaggagaacagatccattccttctgtaccatactgccccatcagcaatccctcctgccatcattcttccttatggtctgatttcggtgagctgttttaatcaaagacacccaatattatggcattattctccaaccttgtcaaaataattgcatacagtaagaacagatggacactgtcatgctaagtaagagtactaactgtagtactccagcatacactatggcacctggcagatcagttgcaccacctgaattaaatgctggatttggatacatacccccaatatcttcaacttaacattacacacacacattggcattcatgcattgtctaaaaattgagtcccatggctgctaataatggttgctactgtcggttggatctgatatgatttctctctttgctgtctcctcagtcttccaggaatggcaggctagtcaaagctcccgaccccatgacccaggtggcaatcaatggctggcccggttggtaaaacctcaccgttttatttaccgtggttactcgcacccgtacgtacactactgttgcccagcctgcacctgcactgcagcagctagacaggcacgagtcagagcagagatctgatctcacctcactcgcctcgatcacattgcctgtctagaagagatgagatgagagcagcaggactgaactctcgccattacaaaatataaaaattttaagattttttttaagtaaagtattgtataagatataagaaaaaacactcttgtgatcactttcggagctaaattttaaatttcttagattttttttatcaggcatcacatttaaactctcaccattttaatatatgactttttagattattttagtaaaatacaaagatatataagaaaaaaaattctcttttagtcactttaggagctgagctaaattttaagttgcttagactttcttgtctgacatctcattggttagaaatatattttcctcccaatggataagctagaaagtgtcccatcggaagctaggtagaacactttgtgacagaggaagtagtataaattttcatgcattgaaatcaataaatatttatattagagtaaaattttaaatcatagaaatgcttccatttgagaacgacgtaggagcatagcagacatttaaagccctttcgaatcgagttataaagaatttcataggattcacgacctacatggaaatttttctaccctttgttacaaagaattggaactttttaaatcccataaaatttttatggaatggtaattgacgtataggttttgaagaaaacttagcaagaatttcaacttcttgaaaatttttctttgagtcgtctctctcattcaactcgtacattttttctgtgatctaatcaaaccaaaaaaaacttagcaagccctttcatatgttttatgtgtcttttaattctctattttacacttcaaatttatctttttatttctacgtttagggcccctttgaatcgtagggttgaaaaaaatagaggaataggaaaaacataggattctgataggaatcgaagtgtaaaacagaggattgcaaaacacaggaatggtcgtttgattggagcgcaggaaaaacgcaggaatcgaatgagagagatagattcaaaggaaaatttttaataggttagagctcttgctaaattttctctaaaatccacatgctatgtgtcatttcataggaatttcataggatttaaaaagcttcaattctttaaatcaaagggccaaataaaaaaaaatttctatagaatttaaatgctataaaatttctacataaatcctgaaaaattaaagcgagagattaggctgaaagatctagagatcctaggtccaaaagaagagagggtcgtgacggtgccgagagccgtcctgccgccctggaaattgtgcctatgtaaagtgtagccggccacacgttgtttcttcctttccttcacatctcacagccccctcttgctgcttccagtccagcccgctcgccgtctcgccattttgaccagaccacctcgctgcctctgctctgctcagtgctgctcactgcctcactcacactgtcacagtgctcttctgggtataagtagctgggcgcgcggcgtcaccttccttggctgcccgtgagcttcccgcgcgcgccatggcacggcctcgtgggcgtgggcgagcttcttggtaggcgaggtgtcgccggcgatcgagttgtctcgagagctactcagctatgaggacggcggccacgccgcctctcgccgccgccgccgccgccgtcgcggcagtgttcctctctgcgttgctgctcgcctccgcctccgcctccgcctccaggctccctcctcctcgccgtcttcttcccctggtacgcgtacgccctcgccgccgccgttcttgatcgacctgagtgttttttaaaaattttgttcggttagttgatgcgctgtgtgttcgtccatggcgatgcgatgcaggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgccgcgcacgacgccgcgccgctcgtggcgacgttcaccaactacaagccgctagggtggaagtgccagtgccgcgaccgcctgttcgacccctgaccctgaggcgcgcgcgccccgtggcgcgccgtggcgtggcgtggcgcgtgcatggcggggctcgcgctcgcgctctcgctgtgaattacggtgtgtgtggccgcgtcgcgcgcgcgcgcgcggcccgtgcccggcagcacatggcgctgcactgctgctgatgctggtggtggtggaatcgttgtcgtcgcgtcgggctgagagggattgttgatagattccgtgtaatatgccaggacaaaattttgtcaccgctgctgctgcccatgcagctggatcggctcggctactctcaccacctatactgtatctttcactggcatctgctcgccgttttggaatctctgcggtgtggggttgcctcttgcatgtacacatgtttttcatgtatcgatccatgcctcctccatggaatctaatgggaatccatcatcgttcatgctggatggatggatggatgtagtgaatggtagtttttcttatttttgttggagatggatattttttactttacatctaatcggatatatgttgtcttttaaattgagaatttagctactcaaacaatccattctgaaattcgttcaaacgaagatttgaacttagtatcttaggctgcgttcggcagactaggttcccaactcctccttattttccgcgcgcacgcttttcaaactactaaacggtgcgtttttacaaaaagtttctatacgaaagttacttaaaaaattaaattaatctatttttgaaaaaaatagctaatatttaattaaatcacgcgctaatcgctactccgttttgcgtgccggggagtgagggttccccgaacacagccttaaagtattattcagatcgctgcaatctacatgccctttcacaaagtgaacggtatagttctagtggtaaattggtaattggtccctctctgcatctgctatgcatgcatgcatttgcttgatagtatgtttttgtgcgttgatgtgcataggtcagttttgttcatttgaggtttcatgccaggga

SEQ ID NO: 5 - DNA, 4429 bp, Oryza nivara
tgccaatatctcttgcctcttggaagttgggactaattgggagctggaagaatagatttcaagaccttattttaagggtatgtttggttgttgcctaacattgtcataactcaatttgccataggtgtggtatggcaaacaaagggtggccttgttggccacaagtgtggcaatgtttagctagaaaatgagtatatgacatataggcccatgaaacaagtggcttgccacaactgaaacaagcaaccaaacgctaagctaacaatctgtggcatgcctaacatgtggtgtggcaatgtgtggcaccaaaccaaacaaccccaaaacgtgcagtgatagtactcctattctcctactgtccaaacccacaaaggtcatcacctgcaccgttgcctgactatttatacaggagtaacaggaccccaagatgacagcatgctactgctccatctagagcgtcgttgttcaccagcagcaaccatcttttcacgctatggcgtagccagagacccagagtacacatctagtgctcctatatgcctcttggtctttgactcgcctccttgcctctctggtctgcagaaccatgcatggaccgaaaggagaacagatccattccttctgtaccatactgccccatcagcaatccctcctgccatcattcttccttatggtctgatttcggtgagctgttttaatcaaagacacccaatattatggcattattctccaaccttgtcaaaataattgcatacagtaagaacagatggacactgtcatgctaagtaagagtactaactgtagtactccagcatacactatggcacctggcagatcagttgcaccacctgaattaaatgctggatttggatacatacccccaatatcttcaacttaacattacacacacacattggcattcatgcattgtctaaaaattgagtcccatggctgctaataatggttgctactgtcggttggatctgatatgatttctctctttgctgtctcctcagtcttccaggaatggcaggctagtcaaagctcccgaccccatgacccaggtggcaatcaatggctggcccggttggtaaaacctcaccgttttatttaccgtggttactcgcacccgtacgtacgctactgttgcccagcctgcacctgcactgcagcagctagacaggcacgagtcagagcagagatctgatctcacctcactcgcctcgatcacattgcctgtctagaagagatgagatgagagcagcaggactgaactctcgccatttcaaaatataaaaattttaagattttttttaagtaaagtattgtataagatataagaaaaaacactcttgtgatcactttcggagctaaattttaaatttcttagattttttttatcaggcatcacatttaaactctcaccattttaatatatgactttttagattattttagtaaaatacaaagatatataagaaaaaaaattctcttttagtcactttaggagctgagctaaattttaagttgcttagactttcttgtctgacatctcattggttagaaatatattttcctcccaatggataagctagaaagtgtcccaacggaagctaggtagaacactttgtgacagagaaagtagtataaattttcatgcattgaaatcaataaatatttatattagagtaaaattttaaatcatagaaatgcttccatttgagaacgacgtaggagcatagcagacttttaaagccctttcgaatcgagttataaagaatttcataggattcacgacctacatggaaatttttctaccctttgttacaaagaattggaactttttaaatcccataaaatttttatggaatggtaattgacgtataggttttgaagaaaacttagcaagaatttcaacttcttgaaaatttttctttgagtcgtctctctcattcaactcgtacattttttctgtgatctaatcaaaccgaaaaaaacttagcaagccctttcatatgttttatgtgtcttttaattctctattttacacttcaaatttatctttttatttctacgtttagggcccctttgaatcgcagggttgaaaaaaatagaagaataggaaaaacataggattctgataggaatcgaagtgtaaaacagaagattgcaaaacacatgaatggtcgtttgattgaagcgcaggaaaaacgcaggaatcgaatgagagagatagattcaaaggaaaattttcaagaggttagagctattgctaaattttctccaaaatccacatgctatgtgtcatttcataggaatttcataggatttgaaaagcttcaattctttgaattaaagggccaaataaaaaaaattctatagaatttaaatcctataaaatttctatataaatcctgaaaaattaaagcgagagattaggctgaaagatctagagatcctaggtccaaaagaagagagggtcgtgacggtgccgagagccgtcctgccgccctggaaattgtgcctatgtaaagtgtagccggccacacgttgtttcttcctttccttcacatctcacagccccgtcttgctgcttccagtccagcccgctcgccgtctcgccattttgaccagaccacctcgctgcctctgctctgctcagtgctgctcactgcctcactcacactgtcacagtgctcttctgggtataagtagctgggcgcgcggcgtcaccttccttggctgcccgtgagcttcccgcgcgcgccatggcacggcctcgtgggcgtgggcgagcttcttggtaggcgaggtgtcgccggcgatcgagttgtctcgagagctactcagctatgaggacggcggccacgccgcctctcgccgccgccgccgccgccgtcgcggcagtgttcctctctgcgttgctgctcgcctccgcctccgcctccgcctccaggctccctcctcctcgccgtcttcttcccctggtacgcgtacgccctcgccgccgccgttcttgatcgacctgagtgttttttaaaaattttgttcggttagttgatgcgctgtgtgttcgtccatggcgatgccgatgcaggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgccgccgccgcgcacgacgccgcgccgctcgtggcgacgttcaccaactacaagccgctagggtggaagtgccagtgccgcgaccgcctgttcgacccctgaccctgaggcgcgcgcgccccgtggcgcgccgtggcgtggcgtggcgcgtgcatggcggggctcgcgctcgcgctctcgctgtgaattacggtgtgtgtggccgcgtcgcgcgcgcgcgcgcggcccgtgcccggcagcacatggcgctgcactgctgctgatgctggtggtggtggaatcgttgtcgtcgcgtcgggctgagagggattgttgatagattccgtgtaatatgccaggacaaaattttgtcaccgctgctgctgcccatgcagctggatcggctcggctactctcaccacctatactgtatctttcactggcatctgctcgccgttttggaatctctgcggtgtggggttgcctcttgcatgtacacatgtttttcatgtatcgatccatgcctcctccatggaatctaatgggaatccatcatcgttcatgctggatggatggatggatgtagtgaatggtagtttttcttatttttgttggagatggatattttttactttatatctaatcggatatatgttgccttttaaattgagaatttagctactcaaacaatccattctgaaattcattcaaacgaagatttgaacttagtatcttaggctgcgttcggcagaccaggctcctaactcctccttattttccgcgcggacgcttttcaaactattaaacggtgcgtttttgcaaaaagtttctatacgaaagttgcttaaaaaattaaattaatctatttttgaaaaaaatagctaatatttaattaaatcacgcgctaatcgctactccgttttgcgtgccagggagtgaggattccccgaacacagccatagagtgttaagtgttattcagatcgctgcaatctacatgcacagccttagagtgttattcagatcgctgcaatctacatgccctttcgcaaagtgaacggtatagttctagtggtaaattggtaattggtccctctctgcatctgctatgcatgcatgcatttgcttgatagtatgtttttgtgcgttgatgtgcataggtcagttttgttcatttgaggtttcatgccaggga

SEQ ID NO: 6 - DNA, 4394 bp, Oryza rufipogon
tgccaatatctcttgcctcttggaagttgggactaattgggagctggaagaatagatttcaagaccttattttaagggtatgtttggttgttgcctaacattgccataactcaatttgccataggtgtggcatggcaaacaaagtgtggccaacaaattattggccacaagtgtggcaatgtttggctagaaaatgagtatataacatgtaggcccatgaaacaagaaagtgtggcttgtcacaactgaaacaagcaaccaaacgctaagctaacaatctgtggcatgcctaacatgtggtgtggcaatgtgtggcaccaaaccaaacaaccccaaaacgtgcagtgatagtactcctattctcctactgtccaaacccacaaaggtcatcacctgcaccgttgcctgactatttatacaggagtaacaggaccccaagatgacagcatgctactgctccatctagagcgtcgttgttcaccagcagcaaccatcttttcacgctatggcgtagccagagacccagagtacacatctagtgctcctatatgcctcttggtctttgactcgcctccttgcctctctggcctgcagaaccatgcatggaccgaaaggagaacagatccattccttctgtaccatactgccccatcagcaatccctcctgccatcattcttccttatggtctgatttcggtgagctgttttaatcaaagacacccaatattatggcattattctccaaccttgtcaaaataattgcatacagtaagaacagatggacactgtcatgctaagtaagagtactaactgtagtactccagcatacactatggcacctggcagatcagttgcaccacctgaattaaatgctggatttggatacatacccccaatatcttcaacttaacattacacacacacacattggcattcatgcattgtctaaaaattgagtcccatggctgctaataatggttgctactgtcggttggatctgatatgatttctctctttgctgtctcctcagtcttccaggaatggcaggctagtcaaagctcccgaccccatgacccaggtggcaatcaatggctggcccggttggtaaaacctcaccgttttatttaccgtggttactcgcacccgtacgtacactactgttgcccagcctgcacctgcactgcagcagctagacaggcacgagtcagagcagagatctgatctcacctcactcgcctcgatcacattgcctgtctagaagagatgagatgagagcagcaggactgaactctcgccatttcaaaatataaaaattttaagattttttttaagtaaagtattgtataagatataagaaaaaaacactcttgtgatcactttcggagctaaattttaaatttcttagatttttttatcaggcatcacatttaaactctcaccattttaatatatgactttttagattattttagtaaaatacaaagatatataagaaaaaaaattctcttttagtcactttaggagctgagctaaattttaagttgcttagactttcttgtctgacatctcattggttagaaatatattttcctcccaatggataagctagaaagtgtcccatcggaagctaggtagaacactttgtgacagaggaagtagtataaattttcatgcattgaaatcaataaatatttatattagagtaaaattttaaatcatagaaatgcttccatttgagaacgacgtaggagcatagcagacttttaaagccctttcgaatcgagttataaagaatttcataggattcacgacctacatggaaatttttctaccctttgttacaaagaattggaactttttaaatcccataaaatttttatggaatggtaattgacgtataggttttgaagaaaacttagcaagaatttcaacttcttgagaatttttctttgagtcgtctctctcattcaactcgtacattttttctgtgatctaatcaaaccgaaaaaaacttagcaagccctttcatatgttttatgtgtcttttaattctctattttacacttcaaatttatctttttatttctacgtttagggcccctttgaatcgcatggttgaaaaaaatagaggaataggaaaaacataggattctgataggaatcgaagtgtaaaacagaagattgcaaaacccaggaatggtcgtttgattgaagcgcaggaaaaatgcaggaatcgaatgagagagatagattcaaaggaaaattttcaagaggctagagctattgctaaattttctccaaaatccacatgctatgtgtcatttcataggaatttcataggatttgaaaagcttcaattctttgaattaaagggccaaataaaaaaaatttctatagaatttaaattctataaaatttctatataaatcctgaaaaattaaagcgagagattaggctgaaagatctagagatcctaggtccaaaagaagagagggtcgtgacggtgccgagagccgtcctgccgccctggaaattgtgcctatgtaaagtgtagccggccacacgttgtttcttcctttccttcacatctcacagccccgtcttgctgcttccagtccagcccgctcgccgtctcgccattttgaccagaccacctcgctgcctctgctctgctcagtgctgctcactgcctcactcacactgtcacagtgctcttctgggtataagtagctgggcgcgcggcgtcaccttccttggctgcccgtgagcttcccgcgcgcgccatggcacggcctcgtgggcgtgggcgagcttcttggtaggcgaggtgtcgccggcgatcgagttgtctcgagagctactcagctatgaggacggcggccacgccgcctctcgccgccgccgccgccgccgtcgcggcagtgttcctctctgcgttgctgctcgcctccgcctccgcctccgcctccaggctccctcctcctcgccgtcttcttcccctggtacgcgtacgccctcgccgccgccgttcttgatcgacctgagtgttttttaaaaattttgttcggttagttgatgcgctgtgtgttcgtccatggcgatgccgatgcaggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgccgccgccgcgcacgacgccgcgccgctcgtggcgacgttcaccaactacaagccgctagggtggaagtgccagtgccgcgaccgcctgttcgacccctgaccctgaggcgcgcgcgccccgtggcgcgccgtggcgtggcgtggcgcgtgcatggcggggctcgcgctcgcgctctcgctgtgaattacggtgtgtgtggccgcgtcgcgcgcgcgccgctgcccgtgcccggcagcacatggcgctgcactgctgctgatgctggtggtggtggaatcgttgtcgtcgcgtcgggctgagagggattgttgatagattccgtgtaatatgccaggacaaaattttgtcaccgctgctgctgcccatgcagctggatcggctcggctactctcaccacctatactgtatctttcactggcatctgctcgccgttttggaatctctgcggtgtggggttgcctcttgcatgtacacatgtttttcatgtatcgatccatgcctcctccatggaatctaatgggaatccatcatcgttcatgctggatggatggatggatgtagtgaatggtagtttttcttatttttgttggagatggatattttttactttacatctaatcggatatatgttgccttttaaattgagaatttagctactcaaacaatccattctgaaattcgttcaaacgaagatttgaacttagtatcttaggctgcgttcggcagaccaggctcccaactcctccttattttccgcgcgcacgcttttcaaactattaaacggtgcgtttttgcaaaaagtttctatacgaaagttgtttaaaaaattaaattaatctatttttgaaaaaaatagctaatatttaattaaatcacgcgctaattgctactccgttttgcgtgccggggagtaaggattccccgaacacagactagagtgttattcagatcgctgcaatctacatgccctttcgcaaagtgaacggtatagttctagtggtaaattggtaattggtccctctctgcatctgctatgcatgcatgcatttgcttgatagtatgtttttgtgcgttgatgtgcataggtcagttttgttcatttgaggtttcatgccaggga

SEQ ID NO: 7 - DNA, 4370 bp, Oryza glaberrima
tgccaatacctcttgcctcttggaagttgggactaattgggagctggaagaatagatttcaagaccttattttaagggtatgtttggttgttgcctaacattgccataactcaatttgccataggtgtggcatggcaaacaaagtgtgttgttggccacaagtgtggcaatgtttggctagaaaatgagtatatgacatgtaggcccatgaaacaagaaagtgtggcttgccacaactgaaacaagcaaccaaacgctaagctaacaatctgtggcatgcttatcatgtggtgtggcaatgtgtggcaccaaaccaaacaaccccaaaacgtgcagtgatagtactcctattctcctactgtccaaacccacaaaggtcatcacctgcaccgttgcctgactatttatacaggagtaacaggaccccaagatgacagcatgctactgctccatctagagcgtcgttgttcaccagcagcaaccatcttttcacgctatggcgtagccagagacccagagtacacatctagtgctcctatatgcctcttggtctttgactcgcctccttcttgcctctctggcctgcagaaccatgcatggaccgaaaggagaacagatccattccttctgtaccatactgccccatcagcaatccctcctgccatcattcttccttatggtctgatttcggtgagctgttttaatcaaagacacccaatattatggcattattctccaaccttgtcaaaataattgcatacagtaagaacagatggacactgtcatgctaagtaagagtactaactgtagtactccagcatacactatggcacctggcagatcagttgcaccacctgaattaaatgctggatttggatacatacccccaatatcttcaacttaacattacacacacacattggcattcatgcattgtctaaaaattgagtcccatggctgctaataatggttgctactgtcggttggatctgatatgatttctctctttgctgtctcctcagtcttccaggaatggcaggctagtcaaagctcccgaccccatgacccaggtggcaatcaatggctggcccggttggtaaaacctcaccgttttatttaccgtggttactcgcacccgtacgtacactactgttgcccagcctgcacctgcactgcagcagctagacaggcacgagtcagagcagagatctgatctcacctcactcgcctcgatcacattgcctgtctagaagagatgagatgagagcagcaggactgaactctcgccattacaaaatataaaaattttaagattttttttaagtaaagtattgtataagatataagaaaaaacactcttgtgatcactttcggagctaaattttaaatttcttagattttttttatcaggcatcacatttaaactctcaccattttaatatatgactttttagattattttagtaaaatacaaagatatataagaaaaaaaattctcttttagtcactttaggagctgagctaaattttaagttgcttagactttcttgtctgacatctcattggttagaaatatattttcctcccaatggataagctagaaagtgtcccatcggaagctaggtagaacactttgtgacagaggaagtagtataaattttcatgcattgaaatcaataaatatttatattagagtaaaattttaaatcatagaaatgcttccatttgagaacgacgtaggagcatagcagacatttaaagccctttcgaatcgagttataaagaatttcataggattcacgacctacatggaaatttttctaccctttgttacaaagaattggaactttttaaatcccataaaatttttatggaatggtaattgacgtataggttttgaagaaaacttagcaagaatttcaacttcttgaaaatttttctttgagtcgtctctctcattcaactcgtacattttttctgtgatctaatcaaaccaaaaaaaacttagcaagccctttcatatgttttatgtgtcttttaattctctattttacacttcaaatttatctttttatttctacgtttagggcccctttgaatcgtagggttgaaaaaaatagaggaataggaaaaacataggattctgataggaatcgaagtgtaaaacagaggattgcaaaacacaggaatggtcgtttgattggagcgcaggaaaaacgcaggaatcgaatgagagagatagattcaaaggaaaatttttaagaggttagagctcttgctaaattttctctaaaatccacatgctatgtgtcatttcataggaatttcataggatttaaaaagcttcaattctttaaatcaaagggccaaataaaaaaaaatttctatagaatttaaatgctataaaatttctacataaatcctgaaaaattaaagcgagagattaggctgaaagatctagagatcctaggtccaaaagaagagagggtcgtgacggtgccgagagccgtcctgccgccctggaaattgtgcctatgtaaagtgtagccggccacacgttgtttcttcctttccttcacatctcacagccccctcttgctgcttccagtccagcccgctcgccgtctcgccattttgaccagaccacctcgctgcctctgctctgctcagtgctgctcactgcctcactcacactgtcacagtgctcttctgggtataagtagctgggcgcgcggcgtcaccttccttggctgcccgtgagcttcccgcgcgcgccatggcacggcctcgtgggcgtgggcgagcttcttggtaggcgaggtgtcgccggcgatcgagttgtctcgagagctactcagctatgaggacggcggccacgccgcctctcgccgccgccgccgccgccgtcgcggcagtgttcctctctgcgttgctgctcgcctccgcctccaggctccctcctcctcgccgtcttcttcccctggtacgcgtacgccctcgccgccgccgttcttgatcgacctgagtgttttttaaaaattttgttcggttagttgatgcgctgtgtgttcgtccatggcgatgcgatgcaggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgccgcgcacgacgccgcgccgctcgtggcgacgttcaccaactacaagccgctagggtggaagtgccagtgccgcgaccgcctgttcgacccctgaccctgaggcgcgcgcgccccgtggcgcgccgtggcgtggcgtggcgcgtgcatggcggggctcgcgctcgcgctctcgctgtgaattacggtgtgtgtggccgcgtcgcgcgcgcgcgcgcggcccgtgcccggcagcacatggcgctgcactgctgctgatgctggtggtggtggaatcgttgtcgtcgcgtcgggctgagagggattgttgatagattccgtgtaatatgccaggacaaaattttgtcaccactgctgctgcccatgcagctggatcggctcggctactctcaccacctatactgtatctttcactggcatctgctcgccgttttggaatctctgcggtgtggggttgcctcttgcatgtacacatgtttttcatgtatcgatccatgcctcctccatggaatctaatgggaatccatcatcgttcatgctggatggatggatggatgtagtgaatggtagtttttcttatttttgttggagatggatattttttactttacatctaatcggatatatgttgtcttttaaattgagaatttagctactcaaacaatccattctgaaattcgttcaaacgaagatttgaacttagtatcttaggctgcgttcggcagactaggttcccaattcctccttattttccgcgcgcacgcttttcaaactactaaacggtgcgtttttgcaaaaagtttctatacgaaagttacttaaaaaattaaattaatctatttttgaaaaaaatagctaatatttaattaaatcacgcgctaatcgctactccgttttgcgtgccggggagtgagggttccccgaacacagccttagagtattattcagatcgctgcaatctacatgccctttcacaaagtgaacggtatagttctagtggtaaattggtaattggtccctctctgcatctgctatgcatgcatgcatttgcttgatagtatgtttttgtgcgttgatgtgcataggtcagttttgttcatttgaggtttcatgccaggga

SEQ ID NO: 8 - DNA, 4371 bp, oryza sativa
tgccaatatctcttgcctcttggaagttgggactaattgggagctggaagaatagatttcaagaccttattttaagggtatgtttggttgcctaacattgccataactcaatttgccataggtgtggcatggcaaacaaagtgtggccaacaaattgttggccacaagtgtggcaatgtttggctagaaaatgagtatataacatgtaggcccatgaaacaagaaagtgtggcttgtcacaactgaaacaagcaaccaaacgctaaactaacaatctgtggcatacctaacatgtggtgtggcaatgtgtggcaccaagccaaacaacccaaaacgtgcagtgatagtactcctattctcctactgtccaaacccacaaaggtcatcacctgcaccgttgcctgactatttatacaggagtaacaggaccccaagatgacagcatgctactgctccatctagagcgtcgttgttcaccagcagcaaccatcttttcacgctatggcgtagccagagacccagagtacacatctagtgctcctatatgcctcttggtctttgactcgcctccttcttgcctctctggcctgcagaaccatgcatggaccggaaggagaacagatccattccttctgtaccatactgccccatcagcaatccctcctgccatcattcttccttatggtctgatttcggtgagctgttttaatcaaagacacccaatattatggcattattctccaaccttgtcaaaataattgcatacagtaagaacagatggacactgtcatgctaagtaagagtactaactgtagtactccagcatacactatggcacctggcagatcagttgcaccacctgaattaaatgctggatttggatacatacccccaatatcttcaacttaacattacacacacacattggcattcatgcattgtctaaaaattgagtcccatggctgctaataatggttgctactgtcggttggatctgatatgatttctctctttgctgtctcctcagtcttccaggaatggcaggctagtcaaagctcccgaccccatgacccaggtggcaatcaatggctggcccggttggtaaaacctcaccgttttatttaccgtggttactcgcacccgtacgtacactactgttgcccagcctgcacctgcactgcagcagctagacaggcacgagtcagagcagagatctgatctcacctcactcgcctcgatcacattgcctgtctagaagagatgagatgagagcagcaggactgaactctcgccatttcaaaatataaaaattttaagattttttttaagtaaagtattgtataagatataagaaaaaaacactcttgtgatcactttcggagctaaattttaaatttcttagattttttttatcaggcatcacatttaaactctcaccattttaatatatgactttttagattattttagtaaaatacaaagatatataagaaaaaaaattctcttttagtcactttaggagctgagctaaattttaagttgcttagactttcttgtctgacatctcattggttagaaatatattttcctcccaatggataagctagaaagtgtcccatcggaagctaggtagaacactttgtgacagaggaagtagtataaattttcatgcattgaaatcaataaatatttatattagagtaaaattttaaatcatagaaatgcttccatttgagaacgacgtaggagcatagcagacttttaaagccctttcgaatcgagttataaagaatttcataggattcacgacctacatggaaatttttctaccctttgttacaaagaattggaactttttaaatcccataaaatttttatggaatggtaattgacgtataggttttgaagaaaacttagcaagaatttcaacttcttgaaaatttttctttgagtcgtctctctcattcaactcgtacattttttctgtgatctaatcaaaccgaaaaaaacttagcaagccctttcatatgttttatgtgtcttttaattctctattttacacttcaaatttatctttttatttctacgtttagggcccttttgaatcgcagggttgaaaaaaatagaggaataggaaaaacataggattctgataggaatcgaagggtaaaacagaagattgcaaaacacaggaatggtcgtttgattgaagcgcaggaaaaacgcaggaatcgaatgagagagatagattcaaaggaaaattttcaagaggctagagctattgctaaattttctccaaaatccacatgctatgtgtcatttcataggaatttcataggatttgaaaagcttcaattctttgaattaaagggccaaataaaaaaaatttctatagaatttaaattctataaaatttctatataaatcctgaaaaattaaagcgagagattaggctgaaagatctagagatcctaggtccaaaagaagagagggtcgtgacggtgccgagagccgtcctgccgccctggaaattgtgcctatgtaaagtgtagccggccacacgttgtttcttcctttccttcacatctcacagccccgtcttgctgcttccagtccagcccgctcgccgtctcgccattttgaccagaccacctcgctgcctctgctctgctcagtgctgctcactgcctcactcacactgtcacagtgctcttctgggtataagtagctgggcgcgcggcgtcaccttccttggctgcccgtgagcttcccgcgcgcgccatggcacggcctcgtgggcgtgggcgagcttcttggtaggcgaggtgtcgccggcgatcgagttgtctcgagaactcagctatgaggacggcggccacgccgcctctcgccgccgccgccgccgccgtcgcggcagtgttcctctctgcgttgctgctcgcctccgcctccgcctccgcctccaggctccctcctcctcgccgtcttcttcccctggtacgcgtacgccctcgccgccgccgttcttgatcgacctgagtgttttttaaaaattttgttcggttagttgatgcgctgtgtgttcgtccatggcgatgccgatgcaggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgccgccgccgcacgacgccgcgccgctcgtggcgacgttcaccaactacaagccgctagggtggaagtgccagtgccgcgaccgcctgttcgacccctgaccctgaggcgcgcgcgccccgtggcgcgccgtggcgtggcgtggcgcgtgcatggcggggctcgcgctcgcgctctcgctgtgaattacggtgtgtgtggccgcgtcgcgtgcccggcagcacatggcgctgcactgctgctgatgctggtggtggtggaatcgttgtcgtcgcgtcgggctgagagggattgttgatagattccgtgtaatatgccaggacaaaattttgtcaccgctgctgctgcccatgcagctggatcggctcggctactttcaccacctatactgtatctttcactggcatctgctcgccgttttggaatctctgcggtgtggggttgcctcttgcatgtacacatgtttttcatgtatcgatccatgcctcctccatggaatctaatgggaatccatcatcgttcatgctggatggatggatggatgtagtgaatggtagtttttcttatttttgttggagatggatattttttactttacatctaatcggatatatgttgtcttttaaattgagaatttagctactcaaacaatccattctgaaattcgttcaaacgaagatttgaacttaagtatcttaggctgcgttcggcagaccaggttcccaactccttattttccgcgcgcacgcttttcaaactattaaacggtgcgtttttgtaaaaagtttctatactaaagttgcttaaaaaattaaattaatctatttttgaaaaaaaatagctaatatttaattaaatcacgcgctaatcgctactccgttttgcgtgccggggagtgagggttccccgaacacagccttagagtgttattcagatcgctgcaatctacatgccctttctcaaagtgaacggtatagttctagtggtaaattggtaattggtccctctctgcatctgctatgcatgcatgcatttgcttgatagtatgtttttgtgcgttgatgtgcataggtcagttttgttcatttgaggtttcatgccaggga

SEQ ID NO: 9 - DNA, 4323 bp, oryza sativa
tgccaatatctcttgcctcttggaagttgggactaattgggagctggaagaatagatttcaagaccttattttaagggtatgtttggttgcctaacattgccataactcaatttgccataagtgtggcaatgtttggctagaaaatgagtatataacatgtaggcccatgaaacaagaaagtgtggcttgtcacaactgaaacaagcaaccaaacgctaaactaacaatctgtggcatacctaacatgtggtgtggcaatgtgtggcaccaagccaaacaacccaaaacgtgcagtgatagtactcctattctcctactgtccaaacccacaaaggtcatcacctgcaccgttgcctgactatttatacaggagtaacaggaccccaagatgacagcatgctactgctccatctagagcgtcgttgttcaccagcagcaaccatcttttcacgctatggcgtagccagagacccagagtacacatctagtgctcctatatgcctcttggtctttgactcgcctccttcttgcctctctggcctgcagaaccatgcatggaccggaaggagaacagatccattccttctgtaccatactgccccatcagcaatccctcctgccatcattcttccttatggtctgatttcggtgagctgttttaatcaaagacacccaatattatggcattattctccaaccttgtcaaaataattgcatacagtaagaacagatggacactgtcatgctaagtaagagtactaactgtagtactccagcatacactatggcacctggcagatcagttgcaccacctgaattaaatgctggatttggatacatacccccaatatcttcaacttaacattacacacacacattggcattcatgcattgtctaaaaattgagtcccatggctgctaataatggttgctactgtcggttggatctgatatgatttctctctttgctgtctcctcagtcttccaggaatggcaggctagtcaaagctcccgaccccatgacccaggtggcaatcaatggctggcccggttggtaaaacctcaccgttttatttaccgtggttactcgcacccgtacgtacactactgttgcccagcctgcacctgcactgcagcagctagacaggcacgagtcagagcagagatctgatctcacctcactcgcctcgatcacattgcctgtctagaagagatgagatgagagcagcaggactgaactctcgccatttcaaaatataaaaattttaagattttttttaagtaaagtattgtataagatataagaaaaaaacactcttgtgatcactttcggagctaaattttaaatttcttagattttttttatcaggcatcacatttaaactctcaccattttaatatatgactttttagattattttagtaaaatacaaagatatataagaaaaaaaattctcttttagtcactttaggagctgagctaaattttaagttgcttagactttcttgtctgacatctcattggttagaaatatattttcctcccaatggataagctagaaagtgtcccatcggaagctaggtagaacactttgtgacagaggaagtagtataaattttcatgcattgaaatcaataaatatttatattagagtaaaattttaaatcatagaaatgcttccatttgagaacgacgtaggagcatagcagacttttaaagccctttcgaatcgagttataaagaatttcataggattcacgacctacatggaaatttttctaccctttgttacaaagaattggaactttttaaatcccataaaatttttatggaatggtaattgacgtataggttttgaagaaaacttagcaagaatttcaacttcttgaaaatttttctttgagtcgtctctctcattcaactcgtacattttttctgtgatctaatcaaaccgaaaaaaacttagcaagccctttcatatgttttatgtgtcttttaattctctattttacacttcaaatttatctttttatttctacgtttagggcccttttgaatcgcagggttgaaaaaaatagaggaataggaaaaacataggattctgataggaatcgaagggtaaaacagaagattgcaaaacacaggaatggtcgtttgattgaagcgcaggaaaaacgcaggaatcgaatgagagagatagattcaaaggaaaattttcaagaggctagagctattgctaaattttctccaaaatccacatgctatgtgtcatttcataggaatttcataggatttgaaaagcttcaattctttgaattaaagggccaaataaaaaaaatttctatagaatttaaattctataaaatttctatataaatcctgaaaaattaaagcgagagattaggctgaaagatctagagatcctaggtccaaaagaagagagggtcgtgacggtgccgagagccgtcctgccgccctggaaattgtgcctatgtaaagtgtagccggccacacgttgtttcttcctttccttcacatctcacagccccgtcttgctgcttccagtccagcccgctcgccgtctcgccattttgaccagaccacctcgctgcctctgctctgctcagtgctgctcactgcctcactcacactgtcacagtgctcttctgggtataagtagctgggcgcgcggcgtcaccttccttggctgcccgtgagcttcccgcgcgcgccatggcacggcctcgtgggcgtgggcgagcttcttggtaggcgaggtgtcgccggcgatcgagttgtctcgagaactcagctatgaggacggcggccacgccgcctctcgccgccgccgccgccgccgtcgcggcagtgttcctctctgcgttgctgctcgcctccgcctccgcctccgcctccaggctccctcctcctcgccgtcttcttcccctggtacgcgtacgccctcgccgccgccgttcttgatcgacctgagtgttttttaaaaattttgttcggttagttgatgcgctgtgtgttcgtccatggcgatgccgatgcaggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgcgccgcgcacgacgccgcgccgctcgtggcgacgttcaccaactacaagccgctagggtggaagtgccagtgccgcgaccgcctgttcgacccctgaccctgaggcgcgcgcgccccgtggcgcgccgtggcgtggcgtggcgcgtgcatggcggggctcgcgctcgcgctctcgctgtgaattacggtgtgtgtggccgcgtcgcgtgcccggcagcacatggcgctgcactgctgctgatgctggtggtggtggaatcgttgtcgtcgcgtcgggctgagagggattgttgatagattccgtgtaatatgccaggacaaaattttgtcaccgctgctgctgcccatgcagctggatcggctcggctactttcaccacctatactgtatctttcactggcatctgctcgccgttttggaatctctgcggtgtggggttgcctcttgcatgtacacatgtttttcatgtatcgatccatgcctcctccatggaatctaatgggaatccatcatcgttcatgctggatggatggatggatgtagtgaatggtagtttttcttatttttgttggagatggatattttttactttacatctaatcggatatatgttgtcttttaaattgagaatttagctactcaaacaatccattctgaaattcgttcaaacgaagatttgaacttaagtatcttaggctgcgttcggcagaccaggttcccaactccttattttccgcgcgcacgcttttcaaactattaaacggtgcgtttttgtaaaaagtttctatactaaagttgcttaaaaaattaaattaatctatttttgaaaaaaaatagctaatatttaattaaatcacgcgctaatcgctactccgttttgcgtgccggggagtgagggttccccgaacacagccttagagtgttattcagatcgctgcaatctacatgccctttctcaaagtgaacggtatagttctagtggtaaattggtaattggtccctctctgcatctgctatgcatgcatgcatttgcttgatagtatgtttttgtgcgttgatgtgcataggtcagttttgttcatttgaggtttcatgccaggga

SEQ ID NO: 10 - DNA, 2965 bp, Oryza longistaminata
tgccaatatctcttgcctcttggaagctgggactaattgggagctggaataatagatttcaagaccttattttaaacgtgcagtgatagtattactcctattctcctactgtccaagcccacaaaggtcatcacctgcaccgttgcctgactatttatacaggagtaacaggaccccaagatgacagcatgctactgctccatctagagcgtcgttgttcaccagcagcaaccatcttttcacgctatggcgtagccagagtacacatctagtgctcctatatgcctcttggtctttgactcgcctccttcttgcctctctggcctgcagaaccatgcatggaccgaaaggagaacagatccattccttctgtaccatatgccccatcagcaatccctcctgccatcattcttccttatggtctaatttcggtgagctgttttaatcaaagacacccaacattatggcattattctccaaccttgtcaaaataattgcatacagtaaaaacagatggacactgtcatgctaagtaagagtactaacagtagtactccagcatacactatggcacctggcagatcagttgcactacctgaattaagtgctggattaggatacatacccccaatatcttcaacttaacattacacacacacattggcattcatggatggtctaaaaattgagtcccatggctgcataatggttgctactgtcggttggatctgatatgatttctctctttgctgtctcctcagtcttccaggaatggcaggctagtcaaagctcccgaccccatgacccaggtggcaatcggagcaaggctaaatagtatagccgacctgttgactatgaagtaagtttttttagtcttctctcattccacttattagagcgagccaatctaatagttcatttatacaatagttaactataaaaatatactacactattaatactcggtcccacctctcatacacacataatatcttggagcccgtgttgcagccggctacaaatctgtagcccgctttctcctctctcttctcttttcttctcgatatatggttatagccggcttatagcatgctattgtacctgctcttatatagttactatctctttacaattaatatagggtctacttgtctctctcacagaattttttggttcttgtgtcccctgcttctcctctcttctctcctccacctcagcattcagccggcttgtaccctactgccactgtacttgctctcaatggctggcccggtcggtaaaacctcaccgttttaccgtggttggtcgcatcttgtccagatatatagccagaaatttgttcttttttgatcggatggagtactattatacttgctctcaatggctggcccggtcggtaaacctcaccgttttaccgtggttgctcgcacccgtacgtacactactgttgcccagcctgcacctgcactgcagcagctagacaggcacgagtcagagcagagatctgatctcacctcactcgcctcgatcacattgcctgtctagaagagatgagatgagagcagcaggactgaactctcaccattttaaaatataagaattttaagattattttttaagaaaagtactgtactacaagatactacgtataagaaaaaacattctcctgtgatcagctaaaatttaaatttctttccctaaaaaaagataaattttaaatttcttagatttttttatcaggcatcacatttaaactctcaccgttttaatatatgactttttagattattttagtaaaatacaaagatatataagaaaaaaaattatcttttagtcactttaggagctgagctaaattttaagttgcttagactttcttgtctgacatctcattggttagaaatatattttcctcccaatggatgagctagaaagtgccccatcggaagctagatagaacactttgtgacagaggaagtagtacaaggctatgtttagttccatccaaagtttggattttggttgaaattgagaatgatgtgactgaaaagttgtgtgtgtatgataggttgatgtgatgggaaaatgcaaagtttggaggcaaactttggatctaaacacaccctaaattttcatgcattgaaatcaataaatatttatattagagtaaaattttaaatcatagaaatgcttccatttgagaacgacgtagtagcatagcagacttttaaagccctaacttcataggattcacgacctacatggaaatttttctaccctttgttacaaagaattggaactttttaaatcccataaaattcttatggaatggtaattgacgtataggttttgaagaaaacttagcaagaatttcaacttcttgaaaaattttctttaagtcgtctctctcattcaactcgtatattttttctgtgatctaatcaaacccaaaaaaacttagcaagccctttcatatgttttatgtgtcttttaattctatattttacacttcaaatttatctttttatttctacgtttagggtgggaaattaaagcgagagattaggctgaaagatctagaaatcctaggtccaaaaaagacagggtcgtgacggtgccgagagccgtcctgccgccctggaaattgtgcctatgtaaagtgtagccggccacacgttgcttcttcctttccttcacatctcacagcccccagtccagctcgctcgccgtctcgccattttgaccagaccacctcgctgcctctgctctgctcagtgctgctcactgcctcactcacactgtcacagtgctcttctgggtataagtagctgggcgcgcggcgtcaccttccttggctgcccgtgagcttcccgcgcgcgccatggcacgggctcgtggccgtgggcgagcttcttggtaggcgaggcgtcggcggcgatcgagttgtctcgagagctactcagct

SEQ ID NO: 11 - DNA, 3150 bp, Oryza longistaminata
tgccaatatctcttgcctcttggaagttgggactaattgggagctggaagaatagatttcaagaccttattttaaacgtgcagtgatagtattactcctattctcctactgtccaaacccacgaaggtcatcacctgcaccgttgcctgactatttatacaggagtaacaggaccccaagatgacagcatgctactgctccatctagagcgtcgttgttcaccagcagcaaccatcttttcacgctatggcgtagccagagtacacatctagtgctcctatatgcctcttggtctttgactcgcctccttcttgcctctctggcctgcagaaccatgcatggaccgaaaggagaacagatccattccttctgtaccatactgccccatcagcaatccctcctgccatcattcttccttgtggtctgatttcggtgagctgttttaatcaaagacacccaatattatggcattattctccaaccttgtcaaaataattgcatacagtaaaaacagatggacactgtcatgctaagtaagagtactaacagtagtactccagcatactccgtactacactatatggcacctgacagatcagttgcaccacctgaattaagtgctggttttggatacatacccccaatatcaacttaacattacacacacacattggcattcatgcatggtctaaaaaattgagtcccatagctgcataagagcaggtacaatagcaggctataagccagctacaaacatattttaagaagataaattaggagagagaagagcagcgggctacagatttgtagccagctgtagcacggacttcaagacacagtgtgtctatgacaggtgggaccaggtattaatagtgtattcagtatgtaactattgtatgaataagctattagattggttatagatgaattgaaactagtactccagttggctatactattgaacttgctctaatggttgctactgtcggttggatctgatatgatttctctctttgctgtctcctcagtcttccaggaatggcaggctagtcaaagctcccgaccccatgacccaggtggcaatcaatggctggcccggttggtaaaacctcaccgtttatttaccgtggttactcgcacccgtacgtacactactgttgcccagcctgcacctgcactgcagcagctagacaggcacgagtcagagcagagatctgatctcacctcactcgcctcgatcacattgcctgtctagaagagatgagatgagagcagcagaactctcaccattttaaaatataagaattttaagattattttttaagaaaagtactgtactacaagatactaatccctccgtccaaaaaaaaaaaaaagcaaactctagatttccgtgtccaattttaactatctgtcttatatgaaatttttttataattcttattttcattgttattagatgataaaacatgattaatattttatgtgtgacttgtctttttaatttttttcataattttttcaaataagacgaacggtcaaacattgggcacggaaatcagggtttgtcttttttttttgggacggagggagtacgtataagaaaaaacattctcttgtgatcagctaaattttaaatttctttccctaaaaaaagataaattttaaatttcttagatttttttatcaggcatcacatttaaactctcaccgttttaatatatgactttttagattattttagtaatatacaaagatatatagaaaaaaaattatcttttagtcactttaggagcttagctaaattttaagttgcttagactttcttgtctgacatctcagggtgtgtttagttgggaaaaggaaattttttagtttcacatcgaacgtttgaccggatgtcggaaggggttttcggatacgaatgaaaaaactaatttcagaactcgcctggaaaccgtgaaacgaatcttttgagactaattaagccgtcattagcacatgtgggttactgtagcacttgtggctaatcacggactaattaggcttaaaagattcgtctcacgatttcccccataactgtgcaattagttttttttatctatatttaatgcttcatgcatatgtccaaagattcgatgtgatgtttttgggaaaaaaatttggagaactaatcgggccctcattggttagaaatatattttcctcccaatggatgaggtagaaagtgccccatcggaagctagatagaacactttgtgacagaggaagtagtataaattttcatgcattgaaatcaataaatatttatattagagtaaaattttaaatcatagaaatgcttccatttgagaacgacgtagtagcatagcagacttttaaagccctttcgaatcgagttataaagaatttcataggattcacgacctacatggaaatttttctaccctttgttacaaagaattggaactttttaaatcccataaaattcttatggaatgataattgacgtataggttttgaagaaaacttagcaagaattttaacttcttgaaaatttttctgagtcgtctctctcattcaactcgtacattttttctgtgatctaatcaaacccaaaaaaaaaacttagcaagccctttcatatgttttatgtgtcttttaattctatattttacacttcaaatttatctttttatttctacgtttagggtgggaaattaaagcgagagattaggctgaaagatctagaaatcctaggtccaaaagaagacagggtcgtgacggtgccgagagccgtcctgccgccctggaaattgtgcctatgtaaagtgtagccggccacacgttgtttcttcctttccttcacatctcacagccccgtctcgctgcttccagtccagcccgctcgccgtctcgccattttgaccagaccacctcgctgcctctgctctgctctgctcagtgctgctcactgcctcactcacactgtcacagtgctcttctgggtataagtagctgggcgcgcggcgtcgccttccttggctgcccgtgagcttccctcgcgcgccatggcacgggctcgtggccgtgggcgagcttcttggtaggcgtcggcggcgatcgagttgtctcgagagctactcagct

SEQ ID NO: 12 - protein, 116 aa, oryza sativa
MRTAATPPLAAAAAAVAAVFLSALLLASASASASRLPPPRRLLPLVGGEVAVAVVAGEEEKVRLGSSPPSCYSKCYGCSPCVAVQVPTLSAPSVPAAAAPRTTPRRSWRRSPTTSR

SEQ ID NO: 13 - protein, 196 aa, oryza sativa
MRTAATPPLAAAAAAVAAVFLSALLLASASASASRLPPPRRLLPLVGGEVAVAVVAGEEEKVRLGSSPPSCYSKCYGCSPCVAVQVPTLSAPSVPAAAAAAARRRAARGDVHQLQAARVEVPVPRPPVRPLTLRRARPVARRGVAWRVHGGARARALAVNYGVCGRVACPAAHGAALLLMLVVVESLSSRRAERDC

SEQ ID NO: 14 - protein, 125 aa, Oryza glaberrima
MRTAATPPLAAAAAAVAAVFLSALLLASASRLPPPRRLLPLVGGEVAVAVVAGEEEKVRLGSSPPSCYSKCYGCSPCVAVQVPTLSAPSVPAAAAAHDAAPLVATFTNYKPLGWKCQCRDRLFDP

SEQ ID NO: 15 - protein, 131 aa, Oryza rufipogon
MRTAATPPLAAAAAAVAAVFLSALLLASASASASRLPPPRRLLPLVGGEVAVAVVAGEEEKVRLGSSPPSCYSKCYGCSPCVAVQVPTLSAPSVPAAAAAAAHDAAPLVATFTNYKPLGWKCQCRDRLFDP

SEQ ID NO: 16 - protein, 131 aa, Oryza nivara; identical to SEQ ID NO: 15

SEQ ID NO: 17 - protein, 129 aa, Oryza barthii
MRTAATPPLAAAAAAVAAVFLSALLLASASASASRLPPPRRLLPLVGGEVAVAVVAGEEEKVRLGSSPPSCYSKCYGCSPCVAVQVPTLSAPSVPAAAAAHDAAPLVATFTNYKPLGWKCQCRDRLFDP

SEQ ID NO: 18 - protein, 126 aa, Oryza glumaepatula
MRTAGTPPLAAAAAAVAAVFLSALLLASASASRLPPPRRLLPLVGGEVAVAVVAGEEEKVRLGSSPPSCYSKCYGCSPCVAVQVPTLSAPSVPAAAAHDAAPLVATFTNYKPLGWKCQCRDRLFDP

SEQ ID NO: 19 - protein, 121 aa, Artificial sequence
  NIL_107B-12 originated from O. longistaminata (IRGC92664)
MRTAATLPLAAVAAVFLSALLLASASASRLPPPRRLLPLVGGEVVVAGEEEKVRLGSSPPSCYSKCYGCSPCIAVQVPTLSAPSVPAAAAAAHDAAPLVATFTNYKPLGWKCQCRDRLFDP

SEQ ID NO: 20 - protein, 126 aa, Oryza longistaminata
MRRAATAPLAAAAAVFLSALLLASASASAFRLPPPRRLLPLVGGEVAVAVAAGEEEKVRLGSSPPSCYSKCYGCSPCVAVQVPTLSAPSVPAAAAAAHDAAPLVATFTNYKPLGWKCQCRDRLFDP

SEQ ID NO: 21 - DNA, 351 bp, oryza sativa
atgaggacggcggccacgccgcctctcgccgccgccgccgccgccgtcgcggcagtgttcctctctgcgttgctgctcgcctccgcctccgcctccgcctccaggctccctcctcctcgccgtcttcttcccctggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgcgccgcgcacgacgccgcgccgctcgtggcgacgttcaccaactacaagccgctag

SEQ ID NO: 22 - DNA, 591 bp, oryza sativa
atgaggacggcggccacgccgcctctcgccgccgccgccgccgccgtcgcggcagtgttcctctctgcgttgctgctcgcctccgcctccgcctccgcctccaggctccctcctcctcgccgtcttcttcccctggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgccgccgccgcacgacgccgcgccgctcgtggcgacgttcaccaactacaagccgctagggtggaagtgccagtgccgcgaccgcctgttcgacccctgaccctgaggcgcgcgcgccccgtggcgcgccgtggcgtggcgtggcgcgtgcatggcggggctcgcgctcgcgctctcgctgtgaattacggtgtgtgtggccgcgtcgcgtgcccggcagcacatggcgctgcactgctgctgatgctggtggtggtggaatcgttgtcgtcgcgtcgggctgagagggattgttga

SEQ ID NO: 23 - DNA, 378 bp, Oryza glaberrima
atgaggacggcggccacgccgcctctcgccgccgccgccgccgccgtcgcggcagtgttcctctctgcgttgctgctcgcctccgcctccaggctccctcctcctcgccgtcttcttcccctggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgccgcgcacgacgccgcgccgctcgtggcgacgttcaccaactacaagccgctagggtggaagtgccagtgccgcgaccgcctgttcgacccctga

SEQ ID NO: 24 - DNA, 396 bp, Oryza rufipogon
atgaggacggcggccacgccgcctctcgccgccgccgccgccgccgtcgcggcagtgttcctctctgcgttgctgctcgcctccgcctccgcctccgcctccaggctccctcctcctcgccgtcttcttcccctggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgccgccgccgcgcacgacgccgcgccgctcgtggcgacgttcaccaactacaagccgctagggtggaagtgccagtgccgcgaccgcctgttcgacccctga

SEQ ID NO: 25 - DNA, 396 bp, Oryza nivara; identical to SEQ ID NO: 24

SEQ ID NO: 26 - DNA, 390 bp, Oryza barthii
atgaggacggcggccacgccgcctctcgccgccgccgccgccgccgtcgcggcagtgttcctctctgcgttgctgctcgcctccgcctccgcctccgcctccaggctccctcctcctcgccgtcttcttcccctggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgccgcgcacgacgccgcgccgctcgtggcgacgttcaccaactacaagccgctagggtggaagtgccagtgccgcgaccgcctgttcgacccctga

SEQ ID NO: 27 - DNA, 381 bp, Oryza glumaepatula
atgaggacggcgggcacgccgcctctcgccgccgccgccgccgccgtcgcggcagtgttcctctctgcgttgctgctcgcctccgcctccgcctccaggctccctcctcctcgccgtcttcttcccctggttggtggcgaggtggcggtggcggtggtggctggggaggaggagaaggtgcggctggggtcgagcccgccgagctgctacagcaagtgctacgggtgcagcccgtgcgtcgcggtgcaggtgcccaccttgtccgccccgtccgtccccgccgccgccgcgcacgacgccgcgccgctcgtggcgacgttcaccaactacaagccgctagggtggaagtgccagtgccgcgaccgcctgttcgacccctga

SEQ ID NO: 28 - DNA, 366 bp, Artificial sequence
  NIL_107B-12 originated from O. longistaminata (IRGC92664)
atgaggacggcggccacgctgcctctcgccgccgtcgccgcggtgttcctctctgcgttgctgctcgcctccgcctccgcctccaggctccctcctcctcgccgtcttcttcccctggttggtggcgaggtggtggtggctggggaggaggagaaggtgcggctaggctcgagcccgccgagctgctacagcaagtgctacgggtgcagcccgtgcatcgcggtgcaggtgcccaccctgtccgccccgtccgtccccgccgccgccgccgccgcgcacgacgccgcgccgctcgtggccacgttcaccaactacaagccgctcgggtggaagtgccagtgccgcgaccgcctgttcgacccctga

SEQ ID NO: 29 - DNA, 381 bp, Oryza longistaminata
atgaggagagcggccacggcgcctctcgccgccgccgccgcggtgttcctctctgcgttgctgctcgcctccgcctccgcctccgccttcaggctccctcctcctcgccgtcttcttcccctggttggtggcgaggtggcggtggcggtggcagctggggaggaggagaaggtgcggctggggtcgagcccgccgagctgctacagcaagtgctacgggtgcagcccgtgcgtcgcggtgcaggtgcccaccctgtccgccccgtccgtccccgccgccgccgccgccgcgcacgacgccgcgccgctcgtggccacgttcaccaactacaagccgctcgggtggaagtgccagtgccgcgaccgcctgttcgacccctga

SEQ ID NO: 30 - DNA, 20 bp, Artificial sequence
  Single strand DNA oligonucleotide
ccaaggacacatatgcatgc

SEQ ID NO: 31 - DNA, 20 bp, Artificial sequence
  Single strand DNA oligonucleotide
aattgttccggtggactcat

SEQ ID NO: 32 - DNA, 21 bp, Artificial sequence
  Single strand DNA oligonucleotide
atgcgtccactcacgaaatgg

SEQ ID NO: 33 - DNA, 20 bp, Artificial sequence
  Single strand DNA oligonucleotide
actccacaaaaggcagttgg

SEQ ID NO: 34 - DNA, 21 bp, Artificial sequence
  Single strand DNA oligonucleotide
tgcccatttttcaattctacg

SEQ ID NO: 35 - DNA, 20 bp, Artificial sequence
  Single strand DNA oligonucleotide
cggagagaaaaggacatgga

SEQ ID NO: 36 - DNA, 20 bp, Artificial sequence
  Single strand DNA oligonucleotide
aggtgtggtggacctacctg

SEQ ID NO: 37 - DNA, 20 bp, Artificial sequence
  Single strand DNA oligonucleotide
actccatcgctttaaggctg

SEQ ID NO: 38 - DNA, 20 bp, Artificial sequence
  Single strand DNA oligonucleotide
ggtgttgtaggttgccgttt

SEQ ID NO: 39 - DNA, 21 bp, Artificial sequence
  Single strand DNA oligonucleotide
cttggagctaattcctgtctc

SEQ ID NO: 40 - DNA, 20 bp, Artificial sequence
  Single strand DNA oligonucleotide
tgagctgttctgcatcctgt

SEQ ID NO: 41 - DNA, 22 bp, Artificial sequence
  Single strand DNA oligonucleotide
tggacctaaatatctgcagcac

SEQ ID NO: 42 - DNA, 20 bp, Artificial sequence
  Single strand DNA oligonucleotide
agcaacgaccatcatttcgt

SEQ ID NO: 43 - DNA, 22 bp, Artificial sequence
  Single strand DNA oligonucleotide
atgtcaagaaaatgagtagacg

SEQ ID NO: 44 - DNA, 19 bp, Artificial sequence
  Single strand DNA oligonucleotide
acgtacggcaaaaggctgt

SEQ ID NO: 45 - DNA, 20 bp, Artificial sequence
  Single strand DNA oligonucleotide
caggatgcattcagtagcag

SEQ ID NO: 46 - DNA, 21 bp, Artificial sequence
  Single strand DNA oligonucleotide
ctactattgctcccaccattc

SEQ ID NO: 47 - DNA, 21 bp, Artificial sequence
  Single strand DNA oligonucleotide
tgatgcgtgtttcatgacaac

SEQ ID NO: 48 - DNA, 20 bp, Artificial sequence
  Single strand DNA oligonucleotide
tgccaatatctcttgcctct

SEQ ID NO: 49 - DNA, 22 bp, Artificial sequence
  Single strand DNA oligonucleotide
acttagcaagccctttcatatg

SEQ ID NO: 50 - DNA, 20 bp, Artificial sequence
  Single strand DNA oligonucleotide
gacaaatcttcgtcgtgagg

SEQ ID NO: 51 - DNA, 21 bp, Artificial sequence
  Single strand DNA oligonucleotide
acgataccatgtttcttcagc

SEQ ID NO: 52 - DNA, 20 bp, Artificial sequence
  Single strand DNA oligonucleotide
gaactgcaagaccctgcatc

SEQ ID NO: 53 - DNA, 20 bp, Artificial sequence
  Single strand DNA oligonucleotide
gattgcatctgcatcactgc

SEQ ID NO: 54 - DNA, 21 bp, Artificial sequence
  Single strand DNA oligonucleotide
gttaactgagcaatgaggact

SEQ ID NO: 55 - DNA, 22 bp, Artificial sequence
  Single strand DNA oligonucleotide
ccaaacatctgattggatttga

SEQ ID NO: 56 - DNA, 20 bp, Artificial sequence
  Single strand DNA oligonucleotide
ctagtgcagaacagaggctt

SEQ ID NO: 57 - DNA, 20 bp, Artificial sequence
  Single strand DNA oligonucleotide
ggttctcatttcctcggttc

SEQ ID NO: 58 - DNA, 20 bp, Artificial sequence
  Single strand DNA oligonucleotide
tcaagatgcacctggtgtct

SEQ ID NO: 59 - DNA, 20 bp, Artificial sequence
  Single strand DNA oligonucleotide
cggagacgaaatcacgtcga

SEQ ID NO: 60 - DNA, 21 bp, Artificial sequence
  Single strand DNA oligonucleotide
gtcatgcaattgtagctaagc

SEQ ID NO: 61 - DNA, 21 bp, Artificial sequence
  Single strand DNA oligonucleotide
ctccatcaatctcgaagaatc

SEQ ID NO: 62 - DNA, 20 bp, Artificial sequence
  Single strand DNA oligonucleotide
gacagggagtgattgaaggc

SEQ ID NO: 63 - DNA, 19 bp, Artificial sequence
  Single strand DNA oligonucleotide
tgggaagaggtggtttcgc

SEQ ID NO: 64 - DNA, 21 bp, Artificial sequence
  Single strand DNA oligonucleotide
cacaagctcgaataaactagc

SEQ ID NO: 65 - DNA, 19 bp, Artificial sequence
  Single strand DNA oligonucleotide
gcaattcatggcgctgttc

SEQ ID NO: 66 - DNA, 21 bp, Artificial sequence
  Single strand DNA oligonucleotide
ttagaatgcaccccatgttct

SEQ ID NO: 67 - DNA, 24 bp, Artificial sequence
  Single strand DNA oligonucleotide
gctagtatatagttcgtacgcacg

SEQ ID NO: 68 - DNA, 20 bp, Artificial sequence
  Single strand DNA oligonucleotide
aatggtccaaggtgtgcatt

SEQ ID NO: 69 - DNA, 20 bp, Artificial sequence
  Single strand DNA oligonucleotide
actaaaccaccatgccgttg

SEQ ID NO: 70 - DNA, 21 bp, Artificial sequence
  Single strand DNA oligonucleotide
gttggaggagctctagaattc

SEQ ID NO: 71 - DNA, 20 bp, Artificial sequence
  Single strand DNA oligonucleotide
ccattgcacaaccttttcct

SEQ ID NO: 72 - DNA, 21 bp, Artificial sequence
  Single strand DNA oligonucleotide
cgtcagaattatggaactgag

SEQ ID NO: 73 - DNA, 20 bp, Artificial sequence
  Single strand DNA oligonucleotide
ctggcaagctactgttttag

SEQ ID NO: 74 - DNA, 20 bp, Artificial sequence
  Single strand DNA oligonucleotide
aaggctcattctgggtcaac

SEQ ID NO: 75 - DNA, 20 bp, Artificial sequence
  Single strand DNA oligonucleotide
tgtcttagcaggtgtgcttg

SEQ ID NO: 76 - DNA, 21 bp, Artificial sequence
  Single strand DNA oligonucleotide
ggctagtacatctgcgtcacg

SEQ ID NO: 77 - DNA, 22 bp, Artificial sequence
  Single strand DNA oligonucleotide
ctttgtaatgttgaatgggagg

SEQ ID NO: 78 - DNA, 23 bp, Artificial sequence
  Single strand DNA oligonucleotide
cacactctgttaccattttacag

SEQ ID NO: 79 - DNA, 20 bp, Artificial sequence
  Single strand DNA oligonucleotide
gacttggatactacggcaag

SEQ ID NO: 80 - DNA, 21 bp, Artificial sequence
  Single strand DNA oligonucleotide
ctgtgaaacacaagcacaagt

SEQ ID NO: 81 - DNA, 21 bp, Artificial sequence
  Single strand DNA oligonucleotide
ctcaggccttatatgtgcatg

SEQ ID NO: 82 - DNA, 19 bp, Artificial sequence
  Single strand DNA oligonucleotide
ggaccagcctagaacagca

SEQ ID NO: 83 - DNA, 20 bp, Artificial sequence
  Single strand DNA oligonucleotide
ggtgaacaacgacgctctag

SEQ ID NO: 84 - DNA, 19 bp, Artificial sequence
  Single strand DNA oligonucleotide
cagcgaggtggtctggtca

SEQ ID NO: 85 - DNA, 20 bp, Artificial sequence
  Single strand DNA oligonucleotide
aggtttggcattgtgcccaa

SEQ ID NO: 86 - DNA, 20 bp, Artificial sequence
  Single strand DNA oligonucleotide
gtcaggagctggtaatgcct

SEQ ID NO: 87 - DNA, 20 bp, Artificial sequence
  Single strand DNA oligonucleotide
cagcgctctttcagatttcg

SEQ ID NO: 88 - DNA, 20 bp, Artificial sequence
  Single strand DNA oligonucleotide
ccacctgaccaacctgtttt

SEQ ID NO: 89 - DNA, 20 bp, Artificial sequence
  Single strand DNA oligonucleotide
cttcgttgcaaggtcggcta

SEQ ID NO: 90 - DNA, 21 bp, Artificial sequence
  Single strand DNA oligonucleotide
ctacttttctccgatacggtc

SEQ ID NO: 91 - DNA, 22 bp, Artificial sequence
  Single strand DNA oligonucleotide
gagtatctcagaacaatcttgg

SEQ ID NO: 92 - DNA, 21 bp, Artificial sequence
  Single strand DNA oligonucleotide
gacacgatttcatcagttcca

SEQ ID NO: 93 - DNA, 21 bp, Artificial sequence
  Single strand DNA oligonucleotide
caagcacagtgcatatagaga

SEQ ID NO: 94 - DNA, 20 bp, Artificial sequence
  Single strand DNA oligonucleotide
gcctctgactagcaatcagc

SEQ ID NO: 95 - DNA, 20 bp, Artificial sequence
  Single strand DNA oligonucleotide
gcttagctttcgcgacgact

SEQ ID NO: 96 - DNA, 20 bp, Artificial sequence
  Single strand DNA oligonucleotide
catatgtatccgctgaacga

SEQ ID NO: 97 - DNA, 18 bp, Artificial sequence
  Single strand DNA oligonucleotide
gttgatttcgccaagggc

SEQ ID NO: 98 - DNA, 22 bp, Artificial sequence
  Single strand DNA oligonucleotide
gcattagcatatcaaatgaacg

SEQ ID NO: 99 - DNA, 19 bp, Artificial sequence
  Single strand DNA oligonucleotide
cgcacgatcgagagatcag

SEQ ID NO: 100 - DNA, 21 bp, Artificial sequence
  Single strand DNA oligonucleotide
cacccgaatccgcctccacca

SEQ ID NO: 101 - DNA, 21 bp, Artificial sequence
  Single strand DNA oligonucleotide
aaggcggaggagacagggagc

SEQ ID NO: 102 - DNA, 22 bp, Artificial sequence
  Single strand DNA oligonucleotide
gagagcgacgcgtcgagcacct

SEQ ID NO: 103 - DNA, 21 bp, Artificial sequence
  Single strand DNA oligonucleotide
gcccactggaccaagctcacc

SEQ ID NO: 104 - DNA, 24 bp, Artificial sequence
  Single strand DNA oligonucleotide
ctcagtgctgctcactgcctcact

SEQ ID NO: 105 - DNA, 23 bp, Artificial sequence
  Single strand DNA oligonucleotide
ccacctcgccaccaacctgcatc

SEQ ID NO: 106 - DNA, 19 bp, Artificial sequence
  Single strand DNA oligonucleotide
cgtcttgctgcttccagtc

SEQ ID NO: 107 - DNA, 24 bp, Artificial sequence
  Single strand DNA oligonucleotide
gcatcaactaaccgaacaaaattt

SEQ ID NO: 108 - DNA, 18 bp, Artificial sequence
  Single strand DNA oligonucleotide
atctcacagcccccagtc

SEQ ID NO: 109 - DNA, 19 bp, Artificial sequence
  Single strand DNA oligonucleotide
gccatggacgcacacagca

SEQ ID NO: 110 - DNA, 32 bp, Artificial sequence
  Single strand DNA oligonucleotide
gaagcttgccaatatctcttgcctcttggaag

SEQ ID NO: 111 - DNA, 31 bp, Artificial sequence
  Single strand DNA oligonucleotide
tgaattccctggcatgaaacctcaaatgaac

SEQ ID NO: 112 - DNA, 20 bp, Artificial sequence
  Single strand DNA oligonucleotide
tggaatcgttgtcgtcgcgt

SEQ ID NO: 113 - DNA, 20 bp, Artificial sequence
  Single strand DNA oligonucleotide
gagcagatgccagtgaaaga

SEQ ID NO: 114 - DNA, 20 bp, Artificial sequence
  Single strand DNA oligonucleotide
tgctccgcattggtcttgac

SEQ ID NO: 115 - DNA, 21 bp, Artificial sequence
  Single strand DNA oligonucleotide
ggagaaactcgagcttgtcga

SEQ ID NO: 116 - DNA, 24 bp, Artificial sequence
  Single strand DNA oligonucleotide
ggcagctcaaggcgcagcagtggg

SEQ ID NO: 117 - DNA, 24 bp, Artificial sequence
  Single strand DNA oligonucleotide
aaaccccactgctgcgccttgagc

SEQ ID NO: 118 - DNA, 24 bp, Artificial sequence
  Single strand DNA oligonucleotide
ggcacaccacggccagctgctcac

SEQ ID NO: 119 - DNA, 24 bp, Artificial sequence
  Single strand DNA oligonucleotide
aaacgtgagcagctggccgtggtg

SEQ ID NO: 120 - DNA, 23 bp, Artificial sequence
  Single strand DNA oligonucleotide
ggcaggcgagcagcaacgcagag

SEQ ID NO: 121 - DNA, 23 bp, Artificial sequence
  Single strand DNA oligonucleotide
aaacctctgcgttgctgctcgcc

SEQ ID NO: 122 - DNA, 106 bp, Artificial sequence
  IR64 partial sequece for alignment
atgaggacggcggccacgccgcctctcgccgccgccgccgccgccgtcgcggcagtgttcctctctgcgttgctgctcgcctccgcctccgcctccgcctccaggc

SEQ ID NO: 123 - DNA, 106 bp, Artificial sequence; identical to SEQ ID NO: 122
  Plant # IRS1493-121 (IR64/IR64)  partial sequencing product for alignment

SEQ ID NO: 124 - DNA, 106 bp, Artificial sequence; identical to SEQ ID NO: 122
  Plant # IRS1493-121 (IR64/IR64)  partial sequencing product for alignment

SEQ ID NO: 125 - DNA, 106 bp, Artificial sequence; identical to SEQ ID NO: 122
  IR64 partial sequence for alignment

SEQ ID NO: 126 - DNA, 105 bp, Artificial sequence
  Plant # IRS1493-033 (IR64/IR64) & IRS1493-062 (IR64/IR64) partial sequencing product for alignment
atgaggacggcggccacgccgcctctcgccgccgccgccgccgccgtcgcggcagtgttcctctcgcgttgctgctcgcctccgcctccgcctccgcctccaggc

SEQ ID NO: 127 - DNA, 102 bp, Artificial sequence
  Plant # IRS1493-033 (IR64/IR64) & IRS1493-062 (IR64/IR64) partial sequencing product for alignment
atgaggacggcggccacgccgcctctcgccgccgccgccgccgccgtcgcggcagtgttcctctcttgctgctcgcctccgcctccgcctccgcctccaggc

SEQ ID NO: 128 - DNA, 94 bp, Artificial sequence
  OL(IRGC 110404) partial sequence for alignment
atgaggagagcggccacggcgcctctcgccgccgccgccgcggtgttcctctctgcgttgctgctcgcctccgcctccgcctccgccttcaggc

SEQ ID NO: 129 - DNA, 87 bp, Artificial sequence
  Plant # IRS1493-041 (OL/OL) partial sequencing product for alignment
atgaggagagcggccacggcgcctctcgccgccgccgccgcggtgttcctctcctgctcgcctccgcctccgcctccgccttcaggc

SEQ ID NO: 130 - DNA, 95 bp, Artificial sequence
  Plant # IRS1493-041 (OL/OL) partial sequencing product for alignment
atgaggagagcggccacggcgcctctcgccgccgccgccgcggtgttcctctcttgcgttgctgctcgcctccgcctccgcctccgccttcaggc

SEQ ID NO: 131 - protein, 116 aa, Artificial sequence; identical to SEQ ID NO: 12
  Nipponbare partial sequence for alignment

SEQ ID NO: 132 - protein, 196 aa, Artificial sequence; identical to SEQ ID NO: 13
  IR64 partial sequence for alignment

SEQ ID NO: 133 - protein, 127 aa, Artificial sequence
  GAD4 partial sequence for alignment
MRTAATPPLAAAAAAAVAAVFLSALLLASASASRLPPPRRLLPLVGGEVAVAVVAGEEEKVRLGSSPPSCYSKCYGCSPCVAVQVPTLSAPSVPAAAAHDAAPLVATFTNYKPLGWKCQCRDRLFDP

SEQ ID NO: 134 - protein, 125 aa, Artificial sequence; identical to SEQ ID NO: 14
  RAE2 partial sequence for alignment

SEQ ID NO: 135 - protein, 126 aa, Artificial sequence; identical to SEQ ID NO: 20
  NIL-91B-42 partial sequence for alignment

SEQ ID NO: 136 - protein, 121 aa, Artificial sequence; identical to SEQ ID NO: 19
  NIL_107B-12  partial sequence for alignment

SEQ ID NO: 137 - DNA, 21 bp, Artificial sequence
  Single strand DNA oligonucleotide
cacccgaatccgcctccacca

SEQ ID NO: 138 - DNA, 21 bp, Artificial sequence
  Single strand DNA oligonucleotide
aaggcggaggagacagggagc

SEQ ID NO: 139 - DNA, 22 bp, Artificial sequence
  Single strand DNA oligonucleotide
gagagcgacgcgtcgagcacct

SEQ ID NO: 140 - DNA, 21 bp, Artificial sequence
  Single strand DNA oligonucleotide
gcccactggaccaagctcacc

SEQ ID NO: 141 - DNA, 24 bp, Artificial sequence
  Single strand DNA oligonucleotide
ctcagtgctgctcactgcctcact

SEQ ID NO: 142 - DNA, 23 bp, Artificial sequence
  Single strand DNA oligonucleotide
ccacctcgccaccaacctgcatc

SEQ ID NO: 143 - DNA, 19 bp, Artificial sequence
  Single strand DNA oligonucleotide
cgtcttgctgcttccagtc

SEQ ID NO: 144 - DNA, 24 bp, Artificial sequence
  Single strand DNA oligonucleotide
gcatcaactaaccgaacaaaattt

SEQ ID NO: 145 - DNA, 18 bp, Artificial sequence
  Single strand DNA oligonucleotide
atctcacagcccccagtc

SEQ ID NO: 146 - DNA, 19 bp, Artificial sequence
  Single strand DNA oligonucleotide
gccatggacgcacacagca

SEQ ID NO: 147 - DNA, 32 bp, Artificial sequence; identical to SEQ ID NO: 110
  Single strand DNA oligonucleotide

SEQ ID NO: 148 - DNA, 31 bp, Artificial sequence; identical to SEQ ID NO: 111
  Single strand DNA oligonucleotide

SEQ ID NO: 149 - DNA, 20 bp, Artificial sequence
  Single strand DNA oligonucleotide
tggaatcgttgtcgtcgcgt

SEQ ID NO: 150 - DNA, 20 bp, Artificial sequence
  Single strand DNA oligonucleotide
gagcagatgccagtgaaaga

SEQ ID NO: 151 - DNA, 20 bp, Artificial sequence
  Single strand DNA oligonucleotide
tgctccgcattggtcttgac

SEQ ID NO: 152 - DNA, 21 bp, Artificial sequence
  Single strand DNA oligonucleotide
ggagaaactcgagcttgtcga

(19) United States
(12) Patent Application Publication
(10) Pub. No.: US 2023/0309480 A1
(43) Pub. Date: Oct. 5, 2023

(54) METHODS OF INCREASING OUTCROSSING RATES IN GRAMINEAE

(71) Applicant: International Rice Research Institute, Los Baños (PH)

(72) Inventors: Sung-Ryul KIM, Ulsan (KR); G.D. PRAHALADA, Sira (IN); Kshirod K. JENA, Cuttack (IN)

(73) Assignee: International Rice Research Institute, Los Baños (PH)

(21) Appl. No.: 18/021,565

(22) PCT Filed: Aug. 18, 2021
(86) PCT No.: PCT/IB2021/057594

§ 371 Date: Feb. 16, 2023

(30) Foreign Application Priority Data
Aug. 18, 2020 (IN) 202021035587

(51) Int. Cl. A01H 1/04; A01H 6/46; A01H 5/10

(52) U.S. Cl. CPC A01H 1/045; A01H 6/4636; A01H 5/10

(57) ABSTRACT

A method of producing a Gramineae plant, the method comprising (a) expressing in a Gramineae plant or plant cell expression of a polynucleotide encoding OLLS1 as set forth in SEQ ID NO: 12 or 13 or a homolog thereof capable of increasing stigma length of the Gramineae plant, wherein when the expressing is by crossing the plant with another plant expressing the polypeptide, selecting for stigma length is performed using markers located between ST87 to ST99; and (b) growing or regenerating the plant.

### RELATED APPLICATION/S

This application claims priority from Indian Patent Application No. 202021035587 filed Aug. 18, 2020, which is incorporated herein by reference in its entirety.

### SEQUENCE LISTING STATEMENT

The ASCII file, entitled 87878 Sequence Listing.txt, created on 17 Aug. 2021, comprising 110,626 bytes, submitted concurrently with the filing of this application is incorporated herein by reference.

### FIELD AND BACKGROUND OF THE INVENTION

The present invention, in some embodiments thereof, relates to methods of increasing outcrossing rates in Gramineae.

Rice is the staple food of more than half the world's population, providing more than 20% of the daily caloric intake of over 3.5 billion people. It is estimated that an additional 116 million tons of rice will be needed by 2035 to feed the world's growing population.

Beginning in the 1940s and 1950s, increasing yields progressively replaced area expansion as the principal source of growth in world grain production. The Green Revolution occurring between the 1940s and late 1960s saw the development of new agricultural practices and technologies that significantly improved grain yield per acre, and is credited with saving millions from mass famine in India during the early 1960s. In particular, the rice variety IR8 was developed, which produced more grain per plant when grown with irrigation and fertilizers. Many additional high-yielding rice lines have been developed since IR8.

Green Revolution technologies, which spurred gains in annual rice yields of more than 3% are now generally considered almost exhausted of any further productivity gains, with annual yield gains falling to around 1.25% since 1990. Decreases in annual gains have lead to plateaus in rice yield in many small to medium-sized countries, including Japan and South Korea. Rice yields in larger countries such as India and China appear to be approaching their own glass ceilings.

Beginning in the early 1970s, significant research efforts have gone into developing hybrid rice, which has been shown to have yields of up to 20% greater than those of conventional Green Revolution high-yielding lines. It was during the early 1970s that Chinese researchers discovered a wild-abortive cytoplasmic male sterile (WA-CMS) rice plant on Hainan Island. This discovery led to development of three-line hybrid rice breeding in China, where hybrid rice has been grown commercially since 1976. This led to Chinese hybrid rice yield surpassing 6.0 t ha⁻¹.

Although hybrid rice has been commercialized on a large scale, particularly in China where hybrid rice covers more than 50% of the total rice-planted area and accounts for about two-thirds of the national production, transferring Chinese hybrid technology to other Asia countries has proven difficult. For hybrid rice commercialization to be successful, hybrid rice seeds must be affordable for farmers, as fresh hybrid seeds are required each season.

Cultivated rice is predominantly self-fertilizing due to the morphology of its flower, i.e., the anthers and stigma are shorter, and pollen is released shortly after the florets open. Outcrossing rates in cultivated rice varieties have diminished along with changes in the morphology of rice flowers during the process of domestication, giving outcrossing rates of about 0.01%. The low rate of outcrossing causes poor hybrid seed production (seed set of 5-20%), resulting in high costs for hybrid rice seeds. These two factors have been cited as major constraints for extending hybrid rice.

It would be beneficial to develop rice varieties and lines with improved outcrossing rates useful for increasing hybrid seed production.

Os08g37890 encoding OsEPFL1 protein was previously identified as GAD1 (GRAIN NUMBER, GRAIN LENGTH AND AWN DEVELOPMENT1) which is originated from O. rufipogon and is associated with grain number per panicle, grain length, and awn development (Jin et al., 2016) and also known as RAE2 (REGULATOR OF AWN ELONGATION 2) which is from African cultivated rice species, O. glaberrima and is involved in awn development (Bessho-Uehara et al., 2016).

Additional Background Art:
Marathi et al. 2014 Euphytica doi:10.1007/s10681-014-1213-2;
Sheeba et al. 2006 Indian J. Agric. Res. 40(4):272-276;
Liu et al. 2015 PLOS ONE|DOI:10.1371;
WO2016/193953;
WO2018/224861;
Bessho-Uehara et al. PNAS Aug. 9, 2016 113 (32) 8969-8974;
Jin et al. Plant Cell, 28, 2453-2463.

### SUMMARY OF THE INVENTION

According to an aspect of some embodiments of the present invention there is provided a method of producing a Gramineae plant, the method comprising:
(a) expressing in a Gramineae plant or plant cell a polynucleotide encoding OLLS1 as set forth in SEQ ID NO: 12 or 13 or a homolog thereof capable of increasing stigma length of the Gramineae plant, wherein when the expressing is by crossing the plant with another plant expressing the polypeptide, selecting for stigma length is performed using markers located between ST87 to ST99; and
(b) growing or regenerating the plant.

According to an aspect of some embodiments of the present invention there is provided a method of identifying a rice plant useful for crossing, the method comprising:
identifying in rice plants at least one marker located between ST87 to ST99 using marker assisted selection (MAS), wherein identification of the at least one marker is indicative of rice plant comprising a stigma length of interest.

According to an aspect of some embodiments of the present invention there is provided a method of producing a Gramineae plant, the method comprising:
(a) expressing in a Gramineae plant or plant cell a polynucleotide encoding OLLS1 as set forth in SEQ ID NO: 12 or 13 or a homolog thereof capable of increasing stigma length of the Gramineae plant; and
(b) growing or regenerating the plant.

According to some embodiments of the invention, the expressing is by genome editing of an endogenous nucleic acid sequence encoding the polypeptide or a cis-acting regulatory region of the nucleic acid sequence.

According to some embodiments of the invention, the expressing is by introducing to the plant a nucleic acid construct comprising a nucleic acid sequence encoding the polypeptide the nucleic acid sequence and/or a cis-acting regulatory element active in plant cells.

According to some embodiments of the invention, the cis-acting regulatory element is of the OLLS1 (SEQ ID NO: 1 or 2).

According to some embodiments of the invention, the cis-acting regulatory element of the OLLS1 is as set forth in SEQ ID NO: 10 or 11.

According to some embodiments of the invention, the marker is selected from the group consisting of ST97, ST87, ST89, ST90, ST91, ST92, ST113, ST93 and ST99.

According to some embodiments of the invention, the marker is ST92 or ST113.

According to some embodiments of the invention, the method further comprises determining stigma length of the plant following the expressing.

According to an aspect of some embodiments of the present invention there is provided a cultivated Gramineae plant being genetically modified to express a polypeptide encoding OLLS1 as set forth in SEQ ID NO: 12 or 13 or a homolog thereof capable of increasing stigma of the plant as compared to the stigma in a plant of same genetic background and developmental stage as the plant and not subjected to the genetic modification, wherein when the genetic modification is an introgression from Oryza longistaminata encoding the polypeptide, the length of the introgression is shorter than 350 or 300 Kb and comprising a marker selected from the group consisting of ST87, ST89, ST90, ST91, ST92, ST113, ST93 and ST99.

According to some embodiments of the invention, the marker is ST92 or ST113.

According to some embodiments of the invention, the marker is ST89.

According to some embodiments of the invention, the plant is cultivated rice.

According to some embodiments of the invention, the plant is cultivated wheat.

According to some embodiments of the invention, the polypeptide is at least 80% identical to an amino acid sequence as set forth in SEQ ID NO: 12 or 13 or wherein the nucleic acid encoding the polypeptide is as set forth in SEQ ID NO: 1 or 2.

According to an aspect of some embodiments of the present invention there is provided a cultivated rice plant comprising an introgression including at least one Oryza longistaminata quantitative trait locus (QTL) associated with stigma length positioned between markers ST87 to ST99 and the introgression being shorter than 350 or 300 Kb.

According to some embodiments of the invention, the introgression is shorter than 100 Kb.

According to some embodiments of the invention, the introgression is shorter than 80 Kb.

According to some embodiments of the invention, the introgression is shorter than 18 Kb.

According to some embodiments of the invention, the introgression is shorter than 10 Kb.

According to some embodiments of the invention, the plant is male sterile.

According to some embodiments of the invention, the plant is environment-sensitive genic male sterile.

According to some embodiments of the invention, the plant is a cytoplasmic male sterile line.

According to some embodiments of the invention, the plant is a maintainer line.

According to some embodiments of the invention, the plant has an out-crossing rate of at least 60%.

According to an aspect of some embodiments of the present invention there is provided a cultivated hybrid Gramineae plant having the plant as a parent or an ancestor.

According to an aspect of some embodiments of the present invention there is provided a processed product comprising DNA of the plant.

According to some embodiments of the invention, the processed product is selected from the group consisting of food feed construction material and paper products.

According to some embodiments of the invention, the processed product is a meal.

According to an aspect of some embodiments of the present invention there is provided an ovule of the plant.

According to an aspect of some embodiments of the present invention there is provided a protoplast produced from the plant.

According to an aspect of some embodiments of the present invention there is provided a tissue culture produced from protoplasts or cells from the cultivated plant, wherein the protoplasts or cells of the tissue culture are produced from a plant part selected from the group consisting of: leaves; pollen; embryos; cotyledon; hypocotyls; meristematic cells; roots; root tips; pistils; anthers; flowers; stems; glumes; and panicles.

According to an aspect of some embodiments of the present invention there is provided a cultivated Gramineae plant regenerated from the tissue culture. wherein the plant is a cytoplasmic male sterile plant having all the morphological and physiological characteristics of the plant.

According to an aspect of some embodiments of the present invention there is provided a method of producing a cytoplasmic male sterile Gremineae plant comprising a long stigma trait of Oryza longistaminata, the method comprising crossing the plant of the stable cytoplasmic male sterile line with a rice plant of a suitable maintainer line of claim 20.

According to an aspect of some embodiments of the present invention there is provided a method for increasing hybrid seed set in a Gramineae plant comprising:
providing a male sterile Gramineae plant comprising a long stigma trait of Oryza longistaminata; and
pollinating the cytoplasmic male sterile plant comprising a long stigma trait of Oryza longistaminata with pollen of a suitable Gramineae line.

According to some embodiments of the invention, the male sterile Gramineae plant is environment-sensitive genic male sterile.

According to some embodiments of the invention, the male sterile Gramineae plant is cytoplasmic genetic male sterile and the suitable Gramineae line is a restorer line.

According to an aspect of some embodiments of the present invention there is provided a method for producing hybrid rice seed comprising:
carrying out the method as described herein; and
collecting hybrid seed set on the cytoplasmic male sterile plant comprising the long stigma trait of Oryza longistaminata.

According to an aspect of some embodiments of the present invention there is provided a method of producing meal, the method comprising:
(a) growing and collecting seeds of the hybrid plant; and
(b) processing the seeds to meal.

According to some embodiments of the invention, the Gramineae plant is selected from the group consisting of cultivated rice, wheat and maize.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C show fine mapping of qSTGL8.0 using three different mapping populations. (a) Initial fine mapping of qSTGL8.0 using the IR64×OL (IRGC110404) cross-derived mapping population. (b) Fine mapping results by using the two additional populations derived from the IR68897B×NIL_107B-12 cross and the IR58025B×NIL_91B-42 cross. Genotypes and phenotypes of the key recombinant plants between PA08-62 and ST05 markers are presented. (c) The annotated genes within the fine-mapped region (˜142 kb) in the reference rice genome, MSU database (www(dot)ricedotplantbiology(dot)msudotedu/). Three candidate genes selected for further validation using transgenic approaches are highlighted by a rectangular.

FIGS. 2A-B show sequence analysis and stigma phenotyping from the CRISPR-Cas9 derived KO plants for the Os08g37890 (OsEPFL1) homologous gene of OL. The CRISPR-Cas9 construct pIRS1493 was transformed to the NIL 6i14-191 possessing qSTGL8.0-OL (IRGC110404) in IR64 background. (a) Sequencing chromatogram near the CRISPR-Cas9 target site of Os08g37890 homologous gene of OL from the two independent T₀ transgenic plants (IRS1493-042 and -001). Both plants possessed the frame-shifted KO alleles caused by ‘T ins/T ins’ in IRS1493-042 plant and ‘T ins/8 bp del’ in IRS1492-001 plant, respectively. The CRISPR-Cas9 target and PAM sequences are marked at the top of the control sequence. (b) Stigma phenotypes of the above KO plants and the control plants (NIL_6i14-191). For simultaneous comparisons, all the stigmas were placed on a single slide glass and scanned.

FIGS. 3A-B show atigma and panicle phenotypes from the complementation T₀ transgenic plants. (a) Stigma phenotype of complementation transgenic lines possessing the 4.4 kb of OLLS1 (IRGC92664) in IR64 background. Stigmas from a tissue-culture derived IR64 control plants (3 plants) and the pIRS1496-derived transgenic plants were scanned together on a single slide. (b) Panicle photos from two complementation test plants and the IR64 control plant. Red arrow: awn, blue arrow: exerted stigma.

FIGS. 4A-B show Phenotypes of awn and grains from the CRISPR-Cas9 derived plants and complementation test transgenic plants. (a) Awn phenotype from the control plant NIL-6i14-191 (left) and CRISPR-Cas9 derived OLLS1 KO plants (right) in T₁ generation. Five uppermost spikelets at the flowering stage were collected from each plant. (b) Grain images from the T₁ generation of complementation test transgenic plants. Presence of the transgene (4.4 kb OLLS1) in the segregating T₁ plants derived from three independent T₀ plants (IRS1496-095, -071, and -076) were identified by ST113 marker and HPT primer set (HPH-979-F/tCaMV-R). In each plant, five uppermost grains of the T₁ plants with transgene (right) and without transgene (left) were collected and were scanned together. The grains from the control plants (IR64) are presented at the bottom.

FIGS. 5A-B Amino acid structure and spatial-temporal gene expression analysis of OsEPFL1/OLLS1. (a) Amino acid structure of OsEPFL1/RAE2/OLLS1 composed of a signal peptide (blue), a propeptide (green), and a mature peptide (pink) based on the previous study (Bessho-Uehara et al., 2016). The cysteine (C) residues in the mature protein are highlighted by red. Signal peptide cleavage site and propeptide cleavage site are marked by arrow (1) and (2) respectively. (b) qRT-PCR analysis of OsEFPL1/OLLS1 from the two NILs (NIL_6i14-191 and NIL_107B-12) and their corresponding backgrounds (IR64 and IR68897B). OsAct1 was used as an internal control. The sequences described in this figure are provided in SEQ ID NOs: 131-136.

FIGS. 6A-C show sequence analysis and stigma phenotyping of a CRISPR-Cas9 derived KO plants for the Os08g37890 (OsEPFL1). (a) Partial CDS of EPFL1 including translation start codon (ATG) and the CRISPR-Cas9 target site of both IR64 and OL (IRGC110404). Sequence variations between two are highlighted by pink. (b) Sequence presentation of OsEPFL1/OLLS1 from the CRISPR-Cas9 derived T₀ plants possessing IR64/IR64 genotype background (IRS1493-121, -033, and -062) and OL/OL genotype background (IRS1493-041). (c) Stigma phenotype with the summary information for the above plants. The sequences described in this figure are provided in SEQ ID NOs: 122-130.

FIG. 7 shows multiple genomic sequences alignment of EPFL1 homologs. Each sequence is corresponding sequence of 4,397 bp of OLLS1 (NIL_107B-12, SEQ ID NO: 2) which was used for complementation test. Protein coding sequences of OLLS1 is underlined and the CRISPR-Cas9 target site with PAM is highlighted by red. The sequence variations among the accessions are highlighted by pink. The OL specific InDel and SNPs in the promoter region are highlighted by green. Nipponbare (SEQ ID NO: 9), IR64 (SEQ ID NO: 8), and O. glaberrima (IRGC96717) (SEQ ID NO: 7) have short stigma (Marathi et al., 2015) and NIL 107B-12 (SEQ ID NO: 2) and OL_IRGC110404 (SEQ ID NO: 1) have a long stigma. Stigma phenotype of the remaining accessions presented above is not available. Source of the sequences: Nipponbare (O. sativa ssp. japonica) from MSU database, IR64 (O. sativa ssp. indica) from Schatz lab (www(dot)schatzlab(dot)cshl(dot)edu/data/rice/) (Schatz et al. 2014), NIL 107B-12 from this study, O. longistaminata (IRGC110404) from the web site (www(dot)olinfres(dot)nig(dot)ac(dot)jp/) (Reuscher et al., 2018), and O. glaberrima (IRGC96717) (SEQ ID NO: 7), O. rufipogon (OR W1943) (SEQ ID NO: 6), O. nivara (IRGC100897) (SEQ ID NO: 5), O. barthii (IRGC105608) (SEQ ID NO: 4), and O. glumaepatula (GEN1233_2) (SEQ ID NO: 3) from the OMAP project (OMAP, www(dot)omap(dot)org/) (Jacquemin et al., 2013).

FIGS. 8A-C Development of a long-exserted stigma lines in the commercial hybrid parental backgrounds, IR68897A/B using OLLS1 gene. (a) The smallest introgression possessing OLLS1 were selected in IR68897B background by precision marker-based breeding and further the OLLS1 was transferred to IR68897A background. (b) Stigma phenotype of the LST972020-VIP02 (A line). (c) Stigma phenotype of the LST972020-VIP34 (A line).

### DESCRIPTION OF SPECIFIC EMBODIMENTS OF THE INVENTION

The present invention, in some embodiments thereof, relates to methods of increasing outcrossing rates in Gramineae.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst conceiving and reducing to practice embodiments of the invention, the present inventors identified a single dominant gene that controls stigma length in rice. This gene is termed OLLS1 after “as Oryza longistaminata long stigma 1”. The following B lines, NIL 91B-42 possessing the qSTGL8.0-OL (IRGC110404) and the NIL 107B-12 possessing the qSTGL8.0-OL (IRGC92664) were crossed with their corresponding recurrent (Re), IR58025B and IR68897B, respectively and the segregation patterns supported a single dominant allele. Fine mapping of the QTL uncovered a 142 kb region on Chromosome 8 between ST97 to ST99. Knock-out of the gene in the NIL-qSTGL8.0 background, using genome editing reverted to a short stigma phenotype. Conversely, horizontal transfer of OLLS1 to indica variety IR64 drastically increased stigma length, by complementation assay. Homologs of OLLS1 include RAE2/GAD1, however the pattern of expression of OLLS1 is unique in that it is strongly expressed in female organ including pistil and stigma. In addition, long-exerted stigma lines were developed by precise introgression of OLLS1 gene (230-350 kb sizes) in the commercial hybrid parental backgrounds, IR68897B/A lines. Taken together the present findings support the use of OLLS1 or small introgressions which comprise it to govern stigma length, which is critical to the development of hybrid seeds.

It will be appreciated that the present teachings contemplate the protection of cultivated Gramineae plant such as cultivated rice plant and will not in any way encompass wild Gramineae per se.

Applicant notes that all varieties designated IR*** (e.g., IR64) not modified according to the present teachings (i.e., so as to have elongated stigma) are not restricted for use.

### Definitions

So that the invention may be more readily understood, certain terms are first defined.

As used herein, the term “plant” refers to an entire plant, its organs (i.e., leaves, stems, roots, flowers etc.), seeds, plant cells, and progeny of the same. The term “plant cell” includes without limitation cells within seeds, suspension cultures, embryos, meristematic regions, callus tissue, leaves, shoots, gametophytes, sporophytes, pollen, and microspores. According to a specific embodiment, the plant is a plant line.

According to a specific embodiment the plant line is an elite line.

The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like. According to a specific embodiment, the plant part comprises the nucleic acid sequence conferring long stigma from Oryza longistaminata. According to a specific embodiment, the plant part is a seed. According to a specific embodiment, the plant part is a hybrid seed.

As used herein, the phrases “progeny plant” refers to any plant resulting as progeny from a vegetative or sexual reproduction from one or more parent plants or descendants thereof. For instance, a progeny plant can be obtained by cloning or selfing of a parent plant or by crossing two parental plants and include selfings as well as the F₁ or F₂ or still further generations. An F₁ is a first-generation progeny produced from parents at least one of which is used for the first time as donor of a trait, while progeny of second generation (F₂) or subsequent generations (F₃, F₄, and the like) are specimens produced from selfings, intercrosses, backcrosses, or other crosses of F₁s, F₂s, and the like. An F₁ can thus be (and in some embodiments is) a hybrid resulting from a cross between two true breeding parents (i.e., parents that are true-breeding are each homozygous for a trait of interest or an allele thereof, e.g., in this case male sterile having long stigma as described herein and a restorer line), while an F₂ can be (and in some embodiments is) a progeny resulting from self-pollination of the F₁ hybrids.

As used herein “cultivated” refers to a Gramineae plant species that has undergone a process of domestication and is therefore endowed with agriculturally desirable characteristics, e.g., higher yield, resistance to biotic/abiotic stress, reproducibility,

As used herein the term “Gramineae plant” refers to the cereal grass family, which cultivated species include but are not limited to wheat, rice, barley, and millet.

According to a specific embodiment the Gramineae plant is a cultivated plant.

As used herein the term “cultivated Oryza plant” refers to a cultivated grass species having a diploid genome, 2n=24 (AA genome). Examples of domesticated Oryza species include but are not limited to, Oryza sativa (Asian rice) or Oryza glaberrima (African rice). The term may be interchanged with the term rice.

Domesticated Oryza varieties contemplated herein according to exemplary embodiments refer to long grain, short grain, white, brown, red and black. These are all art terms known to the skilled artisan.

There are three main subspecies of Oryza sativa:

indica: The indica subspecies is long-grained and mostly grown in tropics and subtropics such as India, Philippines and Vietnam.

japonica: japonica rice is short-grained and high in amylopectin (thus becoming “sticky” when cooked), and is grown mainly in more temperate zone such as Japan and Korea.

javanica: javanica rice is broad-grained and grown in tropical climates.

Other major types include Aromatic and Glutinous.

According to a specific embodiment, the rice subspecies contemplated herein is indica.

According to a specific embodiment, the rice subspecies contemplated herein is japonica.

Within each subspecies and type, there are many cultivars, each favored for particular purposes or regions. Any genetic background of domesticated Oryza e.g., Oryza sativa, can be used. Other varieties and germplasms which can be used according to the present teachings are selected from the group consisting of: IR64; Nipponbare; PM-36, PS 36, Lemont, γS 27, Arkansas Fortuna, Sri Kuning, IR36, IR72, Gaisen Ibaraki 2, Ashoka 228, IR74, NERICA 4, PS 12, Bala, Moroberekan, IR42, Akihikari, IR20, IR56, IR66, NSIC Rc158, NSIC Rc222, and NSIC Rc238, Ciherang, MTU1010, BPT5204, Swarna, Zhenshan97, Minghui63, Irga427, Milyang23, Dongjin, Ilpum.

As used herein the term “wheat” is also interchangeably referred to as “Triticum L.” or “Triticum sub sp”.

As used herein the term “common wheat” is also interchangeably referred to as “Bread wheat” or “Triticum aestivum”.

As used herein the term “durum wheat” is also interchangeably referred to as “Macaroni wheat” or “Triticum durum Desf.” or “Triticum turgidum subsp. durum”.

Wheat is conventionally grown for human or animal food or beverages or as a source of raw materials, food supplements, chemicals or fuel. The common wheat plant is allohexaploid (6N=42) in nature, whereas the durum wheat is a tetraploid (4N=28).

Any genetic background of Triticum can be used. A number of commercial varieties are available including, but not limited to:
T. aestivum (95% of the wheat production. also known as common wheat. typically used for producing flour for baking)
T. aethiopicum (commonly known as Ethiopian wheat)
T. araraticum (commonly known as Armenian or Araratian wild emmer)
T. boeoticum (commonly known as Einkorn wheat)
T. carthhcum (commonly known as Persian wheat)
T. compactum (similar to common wheat)
T. dicoccoides (commonly known as Emmer wheat, Farro, Hulled wheat)
T. dicoccon (commonly known as Emmer wheat, Farro, Hulled wheat)
T. durum
T. ispahanicum (commonly known as Emmer wheat, Farro, Hulled wheat)
T. karamyschevii (commonly known as Emmer wheat, Farro, Hulled wheat)
T. macha
T. militinae
T. monococcum (commonly known as Einkorn wheat)
T. polonicum (commonly known as Polish wheat)
T. spelta (commonly known as Dinkel wheat)
T. timopheevii (commonly known as Zanduri wheat)
T. turanicum
T. urartu (commonly known as Einkorn wheat)
T. vavilovii
T. zhukovskyi

The term “crossed” or “cross” in the context of this invention means the fusion of gametes via pollination to produce progeny (i.e., cells, seeds or plants). The term encompasses both sexual crosses (the pollination of one plant by another) and selfing (self-pollination, i.e., when the pollen and ovule are from the same plant or from genetically identical plants).

“Backcrossing” is a process in which a breeder repeatedly crosses hybrid progeny back to one of the parents, for example, crossing a first generation hybrid F₁ with one of the parental genotypes of the F₁ hybrid. The parent to which the hybrid is backcrossed is the “recurrent parent.”

Marker assisted selection may be used to augment or replace the phenotypic selection (such as by the use of molecular markers of chromosome 8, e.g., ST97, ST87, ST89, ST90, ST91, ST92, ST113, ST93 and ST99).

Regardless of the selection method, following trait selection and backcrossing the genome of the cultivated Gramineae plant e.g., rice plant of the recurrent parent is recovered to at least 85%, at least 87%, at least 90%, at least 92%, at least 94%, at least 96%, or at least 98%. That is, the plant of the invention has a genome being at least 85%, e.g., 85-99.99999999% that of the recurrent parent e.g., Oryza sativa.

According to a specific embodiment, the genome of the recurrent plant (or transgenic plant) comprises no more than 5 genes, 4 genes, 2 genes, or even no more than 1 gene (i.e., OLLS1) of the donor plant e.g., exogenous gene sequences.

As used herein, “outcross” and “outcrossing” refers to cross-pollinations with a plant of differing genetic constitution, as opposed to self-pollination i.e., selfing. Preferably, the two plants are of a same species, sub-species, e.g., rice, e.g., cultivated rice e.g., O. sativa of the same subspecies e.g., japonica, indica etc. However, intercrossing between different Gramineae plant species is also contemplated.

“Outcrossing rate” refers to the rate that a particular plant pollinates or is pollinated by another plant. This is in contrast to self-pollination.

“Improved outcrossing rate” or “increased outcrossing rate” refers to at least 50%, 60%, 70%, 80%, 90%, 100% or even 120%, 130%, 150% 200%, 250%, 300% or even more increase in outcrossing rate as compared to that of a non-converted plant of the same genetic background and of the same developmental stage as growth conditions.

Thus, according to some embodiment of the invention, the cultivated Gramineae plant e.g., rice plant of the invention is endowed with an out-crossing rate which is more than 100% compared non-converted plant.

As used herein the term “heterosis” refers to hybrid vigor, or outbreeding enhancement, that is the improved or increased function of any biological quality in a hybrid offspring. An offspring exhibits heterosis if its traits are enhanced as a result of mixing the genetic contributions of its parents.

According to a specific embodiment, the increased outcrossing rate is manifested by an increase in maximum percent of seed set that can be selected from the group consisting of: a 1.5-fold increase, 2-fold increase, 2.5-fold increase; a 5-fold increase; a 10-fold increase; a 15-fold increase; a 20-fold increase; a 25-fold increase; a 30-fold increase; a 35-fold increase; a 40-fold increase; a 45-fold increase; a 50-fold increase; a 55-fold increase; a 60-fold increase; a 65-fold increase; a 70-fold increase; a 75-fold increase; an 80-fold increase; and an 85-fold increase.

“Yield” describes the amount of grain produced by a plant or a group, or crop, of plants. Yield can be measured in several ways, e.g. t ha⁻¹, and average grain yield per plant in grams.

The term “quantitative trait locus” or “QTL” refers to a polymorphic genetic locus with at least two alleles that reflect differential expression of a continuously distributed phenotypic trait.

As used herein, “introgression” means the movement of one or more genes, or a group of genes, from one plant variety into the gene complex of another as a result of breeding methods (e.g. outcrossing). Introgression also refers to movement of a trait encoded by one or more genes, or a group of genes, from one plant variety into the another.

“Converted” refers to a plant that has been introgressed with a trait of another plant. According to some embodiments, the term refers to a plant introgressed with the long stigma trait of Oryza longistaminata. Introgression of the trait may result from introgression of one or more QTLs associated with the trait. For example a “converted maintainer line” is a maintainer line introgressed with the long stigma trait of Oryza longistaminata.

A plant having “essentially all the physiological and morphological characteristics” of a specified plant refers to a plant having the same general physiological and morphological characteristics, except for those characteristics derived from a particular converted gene or group of genes (e.g., long stigma).

As used herein “stigma length” refers to ‘the total length consisting of brushy and non-brushy parts of the female reproductive organ which is pistil’ A QTL associated with stigma length is abbreviated as “qSTGL”. According to a specific embodiment, a long stigma is about 1.8-2.7 mm (average=2.2 mm)/O. sativa ssp. indica average: is about 1.3 mm/O. sativa ssp. japonica average: is about 0.9 mm/O. glaberrima average: is about 1.1 mm/O. longistaminata average: is about 2.6 mm.

Other QTLs are contemplated herein which can be associated with the improved stigma length. Some are detailed infra.

As used herein “stigma area” refers to ‘the length and breadth of stigma’. A QTL associated with stigma area is abbreviated as “qSTGA”.

As used herein “style length” refers to the length of the stalk (filament) of the bifid stigma. A QTL associated with style length is abbreviated as “qSTYL”.

As used herein “stigma breadth” refers to the distance or measurement from side to side of stigma (brushy) part’. A QTL associated with stigma breadth is abbreviated as “qSTGB”.

As used herein “pistil length” or “total pistil length” which are interchangeably used refers to the total stigma length and style length. Although the word pistil includes ovary, style and stigma, the ovary length is not significantly different between the normal lines and the converted lines, hence, total stigma and style length as pistil length. A QTL associated with pistil length is abbreviated as “qPSTL”.

The term “associated with” or “associated” in the context of this invention refers to, for example, a QTL and a phenotypic trait (e.g., long stigma), that are in linkage disequilibrium, i.e., the QTL and the trait are found together in progeny plants more often than if the nucleic acid and phenotype segregated independently.

The term “marker” or “molecular marker” or “genetic marker” refers to a genetic locus (a “marker locus”) used as a point of reference when identifying genetically linked loci such as a QTL.

A “probe” is an isolated nucleic acid to which is attached a conventional detectable label or reporter molecule, e.g., a radioactive isotope, ligand, chemiluminescent agent, or enzyme. Such a probe is complementary to a strand of a target nucleic acid, in the case of the present invention, to a strand of genomic DNA of the long stigma introgression from Oryza longistaminata, whether from a Gramineae plant e.g., rice plant or from a sample that includes DNA from the Gramineae plant e.g., rice plant (e.g., meal). Probes according to the present invention include not only deoxyribonucleic or ribonucleic acids but also polyamides and other probe materials that bind specifically to a target DNA sequence and can be used to detect the presence of that target DNA sequence.

“Primers” are isolated nucleic acids that are annealed to a complementary target DNA strand by nucleic acid hybridization to form a hybrid between the primer and the target DNA strand, then extended along the target DNA strand by a polymerase, e.g., a DNA polymerase. Primer pairs of the present invention refer to their use for amplification of a target nucleic acid sequence, e.g., by the polymerase chain reaction (PCR) or other conventional nucleic-acid amplification methods.

Probes and primers are generally 11 nucleotides or more in length, preferably 18 nucleotides or more, more preferably 24 nucleotides or more, and most preferably 30 nucleotides or more. Such probes and primers hybridize specifically to a target sequence under high stringency hybridization conditions. According to some embodiment, probes and primers according to the present invention have complete sequence similarity with the target sequence, although probes differing from the target sequence and that retain the ability to hybridize to target sequences may be designed by conventional methods.

Methods for preparing and using probes and primers are described, for example, in Molecular Cloning: A Laboratory Manual, 2nd ed., vol. 1-3, ed. Sambrook et al., Cold Spring Harbor Laboratory Press, Cold Spring Harbor, N.Y., 1989 (hereinafter, “Sambrook et al., 1989”); Current Protocols in Molecular Biology, ed. Ausubel et al., Greene Publishing and Wiley-Interscience, New York, 1992 (with periodic updates) (hereinafter, “Ausubel et al., 1992”); and Innis et al., PCR Protocols: A Guide to Methods and Applications, Academic Press: San Diego, 1990. PCR-primer pairs can be derived from a known sequence, for example, by using computer programs intended for that purpose such as Primer (Version 0.5, .COPYRGT. 1991, Whitehead Institute for Biomedical Research, Cambridge, Mass.).

Exemplary primers for detecting ST97, ST87, ST89, ST90, ST91, ST92, ST113, ST93 and ST99 and other markers are provided in Table 5 hereinbelow which is considered as an integral part of the embodiments of the invention.

The term “specific for (a target sequence)” indicates that a probe or primer hybridizes under stringent hybridization conditions only to the target sequence in a sample comprising the target sequence.

As used herein, “amplified DNA” or “amplicon” refers to the product of nucleic-acid amplification of a target nucleic acid sequence that is part of a nucleic acid template.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The term “isolated” refers to at least partially separated from the natural environment e.g., from a plant cell.

As used herein “homologous” or “orthologous” sequences refer to naturally occurring or synthetic nucleic acid sequences (or polypeptides encoded thereby) which comprise at least the functional portion of the polynucleotides/polypeptides of the invention e.g., OLLS1 of Oryza longistaminata, and are capable of imparting a plant with the long stigma trait.

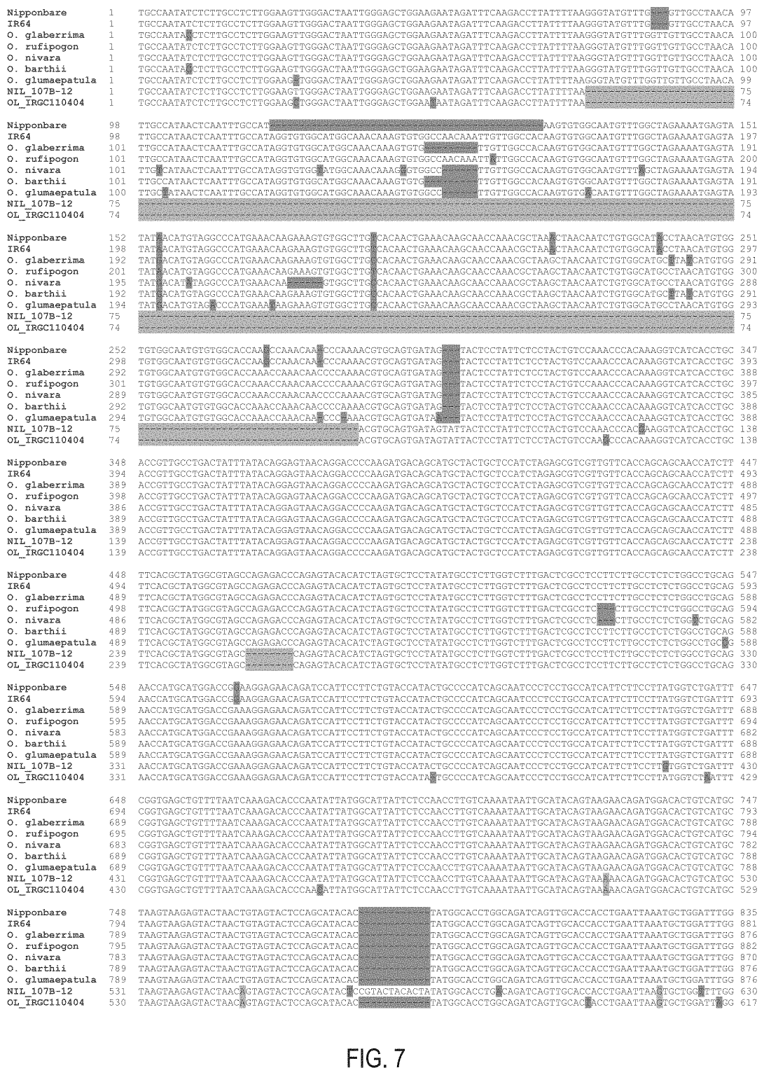

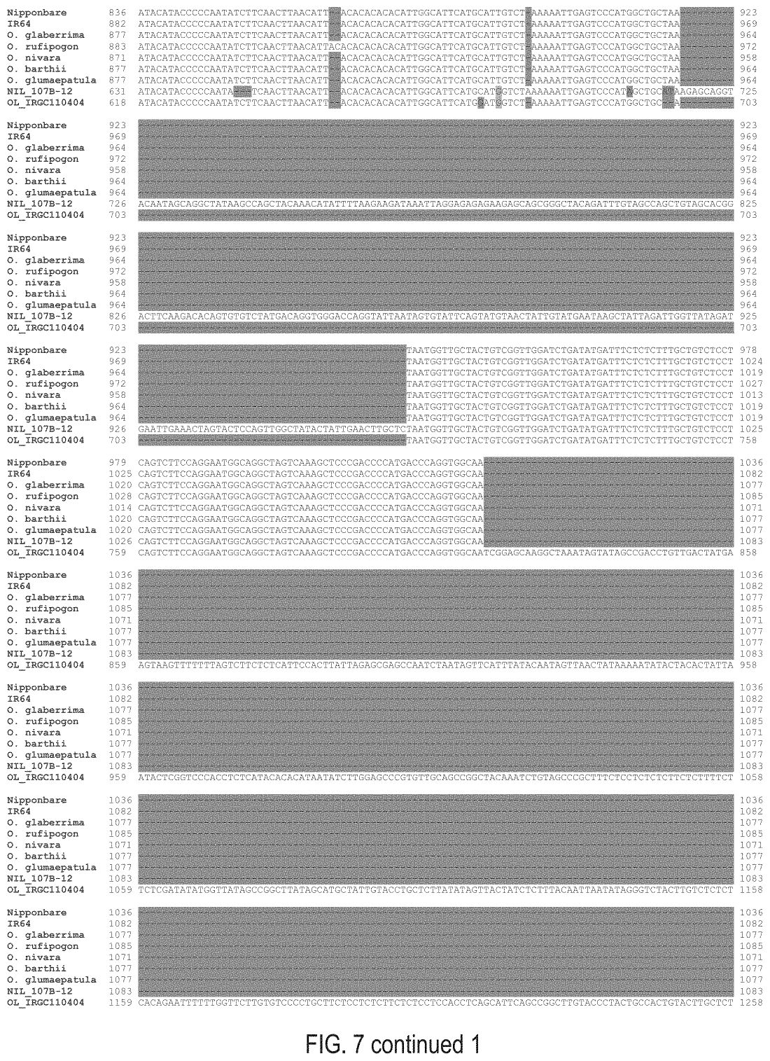

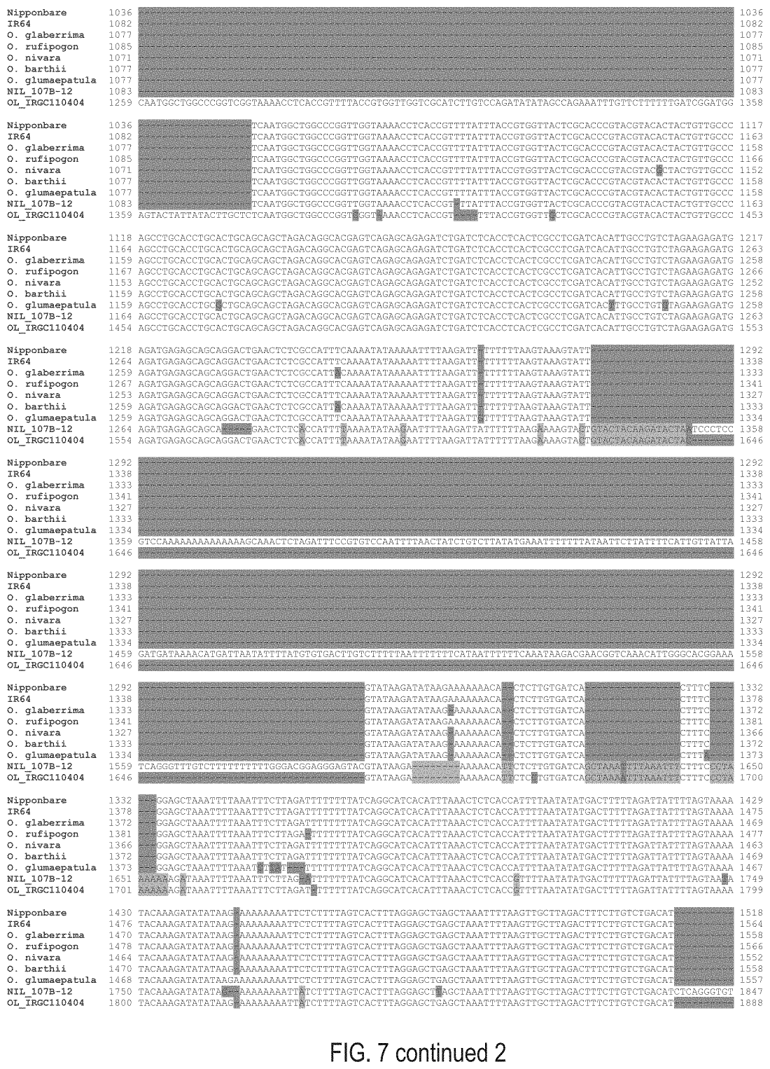

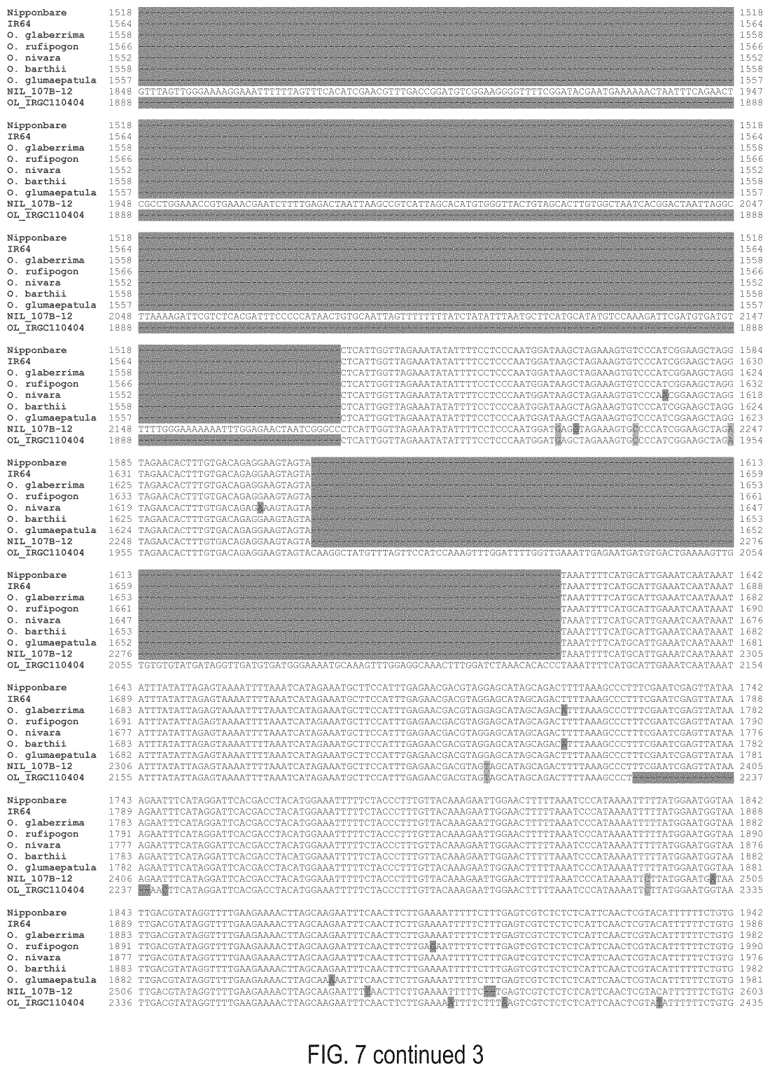

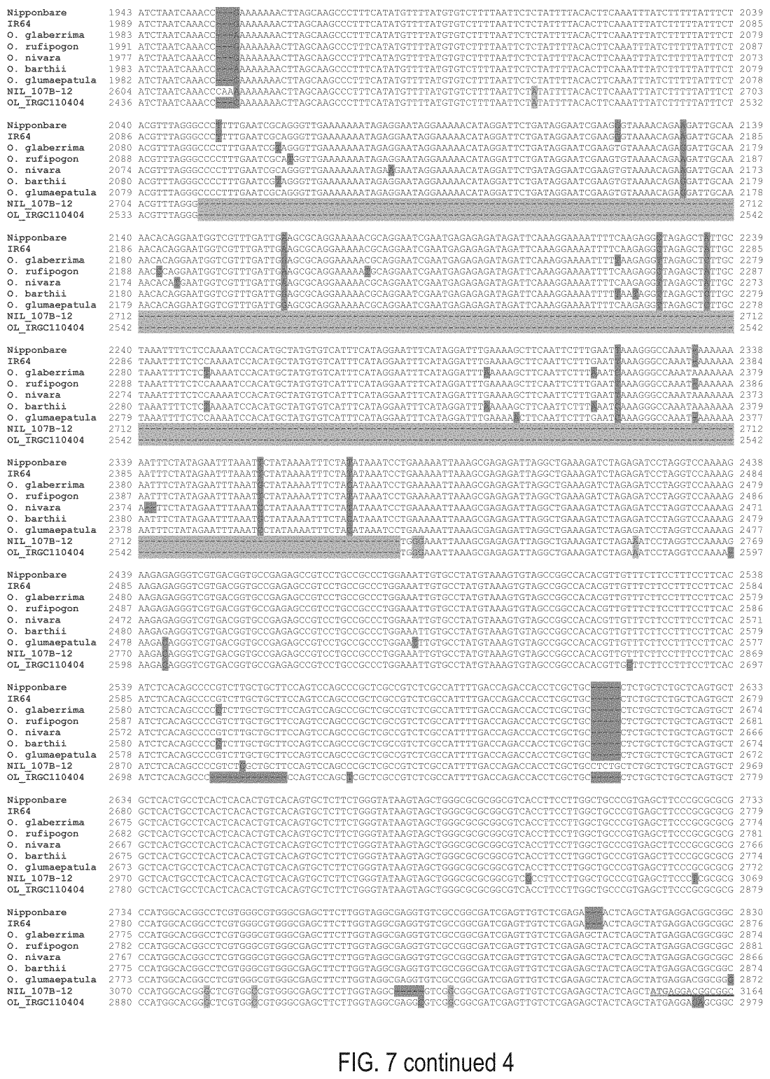

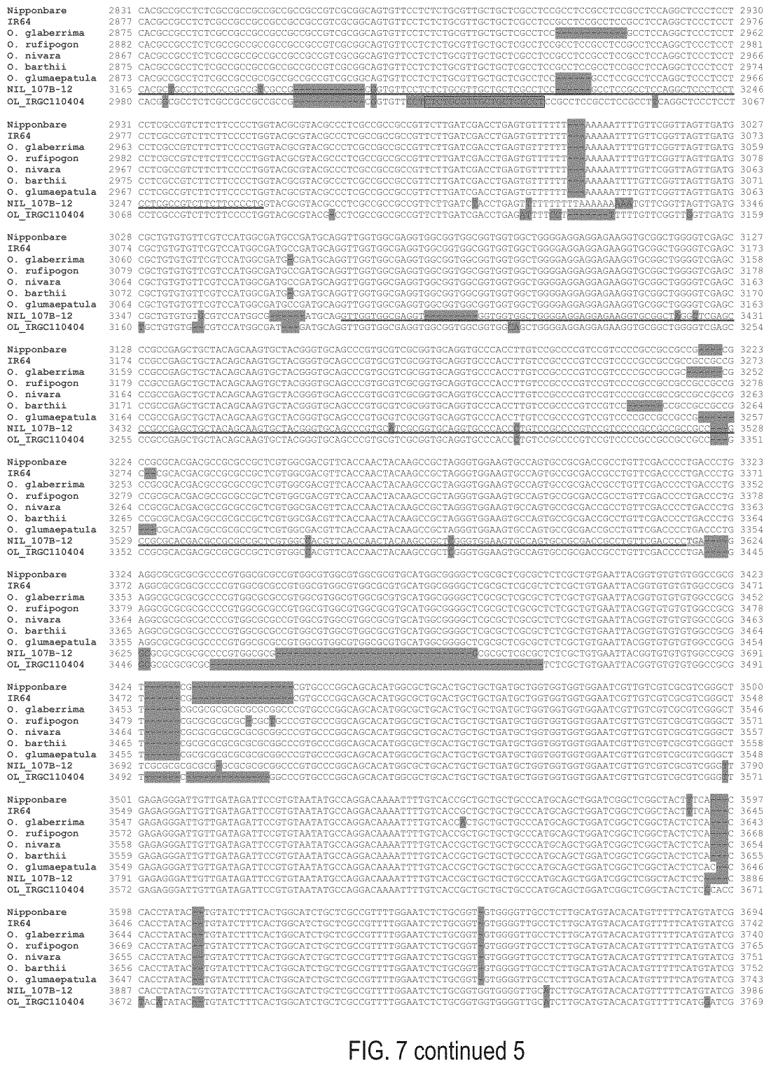

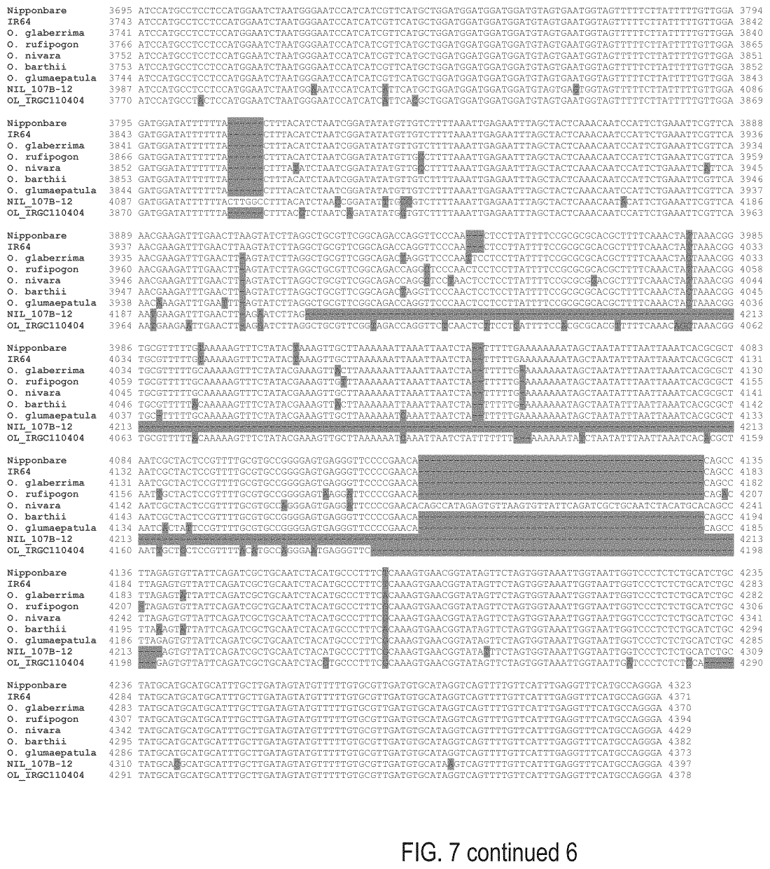

Such homologues or orthologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to SEQ ID NOs: 1-9 see FIG. 7), as determined using the BestFit software of the Wisconsin sequence analysis package, utilizing the Smith and Waterman algorithm and default parameters.

General Description

Heterosis (also called as hybrid vigour) is the phenomenon in which F₁ hybrids derived from diverse parents show superiority over their parents by displaying higher yield, higher levels of disease resistance, higher levels of pest resistance, increased vigor, higher number of spikelets per panicle, higher number of productive tillers, etc. Heterosis is available in the first generation only because of genotypical and phenotypical uniformity among F₁s. And while farmers tend to use a lower seed rate for hybrids than for conventional inbred varieties because of their better seed quality relative to non-hybrids, it is necessary to purchase fresh seeds every season. The added expense of hybrid seeds, especially the difficulty to produce hybrid seed (e.g., rice), often puts the seed out of reach of the farmers.

By way of example (however, this can be broadened to any Gramineae), hybrid rice is developed by exploiting the phenomenon of heterosis. Rice, being a strictly self-pollinated crop, requires the use of a male sterility system to develop commercial rice hybrids. Male sterility (genetic or nongenetic) makes the pollen of the plant unviable, so that rice spikelets are incapable of setting seeds through selfing. A male sterile line is used as a female parent, and grown next to a pollen donor parent in an isolated plot to produce a bulk quantity of hybrid seed resulting from cross pollination from the pollen donor parent. The seed set on the male sterile plants is the hybrid seed that is used to grow the commercial hybrid crop.

The three-line method of hybrid rice breeding is based on cytoplasmic male sterility (CMS) and the fertility restoration system, and involves three lines: the CMS line (A line); maintainer line (B line), and restorer (pollinator; R line).

Male sterility is controlled by the interaction of a genetic factor S present in the cytoplasm and nuclear gene(s). The male sterility factor S is located in the mitochondrial genome. The A line is male sterile when the male sterility-controlling factor S in the cytoplasm (mitochondria genome) and the non-functional recessive alleles (rf) of fertility-restoring genes are present in the nucleus genome. The maintainer line (B line) is iso-cytoplasmic to the CMS line since it has the same genotype of nuclear genome with A line but differs in cytoplasmic factor (N), which makes it self-fertile, so it has the capacity to maintain the sterility of the A line when crossed with it. A restorer (R line) possesses dominant fertility-restoring genes (Rf) and it is dissimilar to or diverse from the A line. Crossing a restorer line as a pollen parent with a CMS (A) line as a female parent restores the fertility in the derived F₁ hybrid, allowing plants grown from the hybrid seed to self pollinate and set seed.

Hybrid seed production using the CMS-based three-line method involves two basic steps: multiplication of the CMS line and production of hybrid seeds. Multiplication of the CMS line with its maintainer line by outcrossing by hand for a small quantity of seed, or in the field under isolation by space or time to produce bulk quantity of seed. For production of the CMS line, it is grown, for example, in six or eight rows interspersed by two rows of maintainer line in an alternating manner.

Because there usually small differences between the growth duration of A and B lines, their sowing dates can be adjusted to achieve good synchronization of their flowering. Several other techniques (including but not limited to flag-leaf clipping, gibberellic acid application, and supplementary pollination by rope pulling or shaking) are used to improve the outcrossing rate and seed yield of the CMS line.

The production of hybrid seeds involves the use of CMS lines with a selected restorer line (pollinator; R line) by growing them in a specific female:male ratio in the field under isolation by space or time. The sowing dates of A and R lines are preferably staggered to achieve synchronization of their flowering. As in the maintenance step, outcrossing rate and hybrid set may be increased by methods including but not limited to flag-leaf clipping, gibberellic acid application, and supplementary pollination by rope pulling or shaking.

Higher seed setting in CMS line is very crucial for cost-effective hybrid seed production. Basically the female organ of each spikelet from the CMS line (A line) must capture fertile pollen grains from the B or R line plants to set seed. A long-exerted stigma trait is considered as a priority target trait for this. The extent of outcrossing in the female parent (CMS line) is influenced by floral traits.

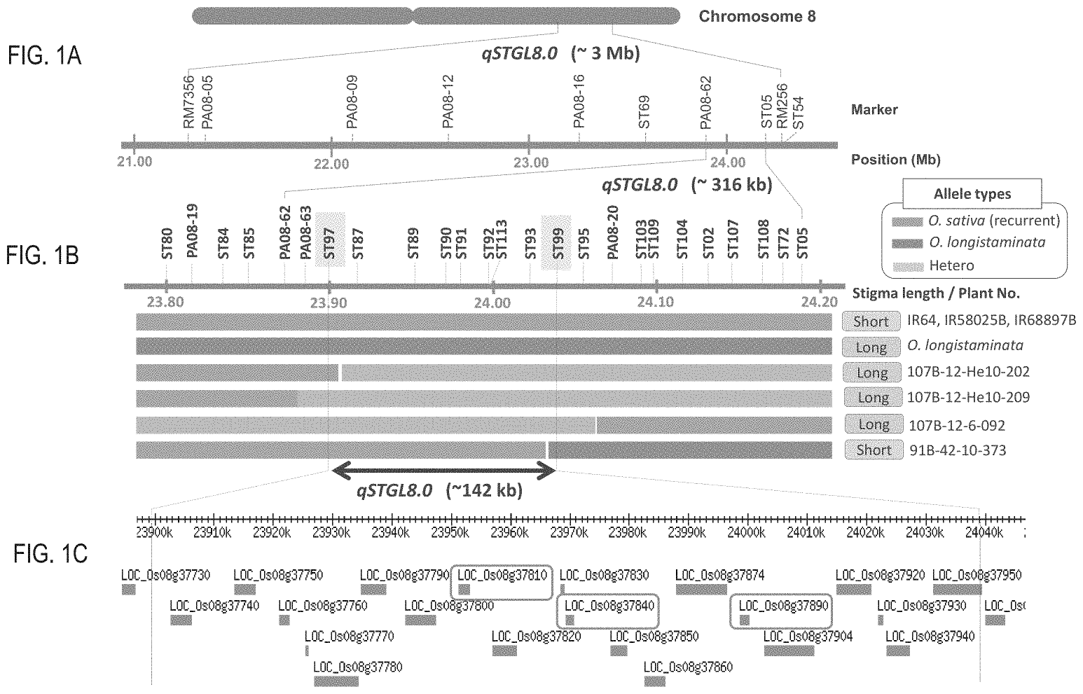

Oryza longistaminata (e.g., OF NIL 107B-12 or OL-IRGC110404) is first crossed with a maintainer line, thereby introgressing the long and exserted stigma trait into one or more plants of the maintainer line. Any maintainer line can be crossed with the NIL 107B-12 or Oryza longistaminata. In particular embodiments, the two popular indica maintainer lines IR58025B and IR68897B are crossed with Oryza longistaminata, thereby introgressing the long and exserted stigma trait into at least one plant of the maintainer line. Progeny are selected for long stigma in F₁, BC₁F₁, BC₂F₁, and their segregating generations. FIG. 1 (top panel) of WO2018/224861 depicts the general strategy for introgressing the long and wide stigma trait of Oryza longistaminata into a maintainer line.

In one embodiment, F₁ progeny are backcrossed with a rice plant of the maintainer line to produce a BC₁F₁ generation. Fertile BC₁F₁ with increased stigma length relative to rice plants of the maintainer line are selected for backcrossing. Backcrossing with the recurrent parent can be done 1 to 5 times, producing BC₂F₁ to BC₆F₁ progeny rice plants. Fertile progeny are again selected, where selected plants have all the physiological and morphological characteristics of the maintainer line, except for the desired trait of increased stigma length. Selected plants are intercrossed or selfed to produce F₂ or later generations, which are stable for the long stigma trait. Those skilled in the art will recognize that modifications to this general strategy may be made, but still result in a converted maintainer line. Such modifications are to be recognized as being within the scope of the present invention.

In certain embodiments, progeny plants of a cross between Oryza longistaminata and the maintainer line, or early backcross progeny, are produced via embryo rescue.

The long and exserted stigma trait is then introgressed into a cytoplasmic male sterile (CMS) line by crossing the CMS line with a corresponding maintainer line, wherein the corresponding maintainer line expresses the long and exserted stigma trait derived from Oryza longistaminata (i.e., converted). For example, CMS line IR58025A is crossed with selected IR58025B progeny from the cross with Oryza longistaminata, where the selected progeny express the long and exserted stigma trait. CMS line IR68897A is crossed with long and exserted stigma-introgressed maintainer line IR68897A. By using marker-assisted breeding, other CMS lines can be similarly crossed with selected plants of an appropriate maintainer line, where the selected plants express the long and exserted stigma trait of Oryza longistaminata. Progeny of the CMS x converted maintainer line are selected for long and exserted stigma. In certain embodiments, fertile F₁ progeny with long stigma are backcrossed with the CMS recurrent parent line, followed by backcrossing fertile BC₁F₁ progeny with long stigma with the CMS recurrent parent. Backcross progeny with complete male sterility and long stigma are selected. In some embodiments, backcross progeny with complete male sterility and long stigma are selected for generating a stable CMS line having long stigma. The stable CMS line is preferably generated by backcrossing. FIG. 1 (bottom panel) of WO2018/224861 depicts the general strategy for introgressing the long and exserted stigma trait of Oryza longistaminata, first introduce into the maintainer line, into a CMS line. Those skilled in the art will recognize that modifications to this general strategy may be made (e.g., additional backcrossing), but still result in a converted CMS line. Such modifications are to be recognized as being within the scope of the present invention.

In certain embodiments of the breeding methods described above, increased stigma length is selected when stigma length is at least 30% greater, at least 40% greater, at least 50% greater, or at least 60% greater than stigma length of rice plants of the maintainer line not introgressed with the long stigma trait of Oryza longistaminata. In a preferred embodiment, increased stigma length is selected when stigma length is at least 50% greater than stigma length of rice plants of the maintainer line not introgressed with the long stigma trait of Oryza longistaminata.

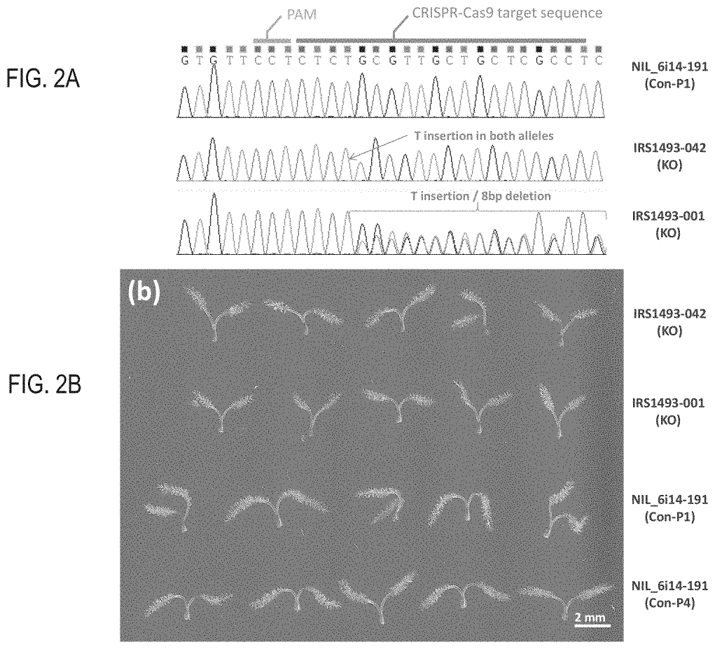

Converted CMS lines are then pollinated by a restorer line comprising a dominant fertility-restoring genes (FIG. 2 of WO2018/224861). Any restorer line capable of restoring fertility in the converted CMS can be used. In one embodiment, the restorer line is IR71604-4-4-4-2-2-2R. Hybrid seed resulting from the converted CMS x restorer cross is set on plants of the converted CMS line. The hybrid seed is then collected for future planting. In particular embodiments, the converted CMS line, restorer line, or both, comprise one or more desirable agronomic characteristics. Desirable agronomic characteristics include, but are not limited to semi-dwarf plant height, high yield, uniformity, bacterial leaf blight disease resistance, brown planthopper pest resistance, and/or drought tolerance. In a preferred embodiment, rice grown from hybrid seed set on converted CMS lines described herein outperforms its parents in at least one desirable agronomic characteristic. For example, hybrid seeds described herein can result in higher yield, higher uniformity, higher levels of disease resistance, higher levels of pest resistance, and/or improved drought tolerance.

It will be appreciated that the present teachings can be also implemented for producing hybrid Gramineae seeds (e.g., rice) using the two-line system, which utilizes photoperiod- and thermo-sensitive genic male-sterile lines (PGMS and TGMS, respectively) and male parental lines (thus named the “second-generation” hybrid rice). The first environment-sensitive genic male sterile (EGMS) rice mutant line was discovered by Prof Mingsong Shi [Cheng. J. Zhuang, Y Fan, J. Du, L. CaoProgress in research and development on hybrid rice: a super-domesticate in China Ann Bot, 100 (2007), pp. 959-966]. In commercial production, the fertility of PGMS and TGMS lines is “switched on” for self-propagation and “switched off” for hybrid seed production by changing the conditions (e.g., locations) where the plants are grown. Compared to the “three-line” hybrid system, the “two-line” hybrid system is easier to operate and more efficient in utilization of rice germplasm, and it produces hybrids of higher yields and better grain quality.

Other contemplated EGMS lines include, but are not limited to Reverese TGMS (rTGMS), PTGMS and rPGMS.

Thus, according to an aspect of the invention there is provided a method of producing a Gramineae plant, the method comprising:
(a) expressing in a Gramineae plant or plant cell a polynucleotide encoding OLLS1 as set forth in SEQ ID NO: 12 or 13 or a homolog thereof capable of increasing stigma length of the Gramineae plant, wherein when said expressing is by crossing the plant with another plant expressing said polypeptide, selecting for stigma length is performed using a marker located between ST87 to ST99; and
(b) growing or regenerating the plant.

According to an additional or an alternative aspect there is provided a method of identifying a rice plant useful for crossing, the method comprising:
identifying in rice plants at least one marker located between ST87 to ST99 using marker assisted selection (MAS), wherein identification of said at least one marker is indicative of rice plant comprising a stigma length of interest.

According to an additional or an alternative aspect there is provided a method of producing a Gramineae plant, the method comprising:
(a) expressing in a Gramineae plant or plant cell a polynucleotide encoding OLLS1 as set forth in SEQ ID NO: 12 or 13 or a homolog thereof capable of increasing stigma length of the Gramineae plant; and
(b) growing or regenerating the plant.

As used herein “OLLS1” refers to the gene and optionally product thereof which controls stigma length. The OLLS1 is encoded by SEQ ID NO: 1 or 2 and is associated with the molecular marker ST113 (in the promoter region of the gene). About 16 Kb apart is the molecular marker ST92.

Natural homologs of the gene are available e.g., GAD1 (GRAIN NUMBER, GRAIN LENGTH AND AWN DEVELOPMENT1) which is originated from O. rufipogon and is associated with grain number per panicle, grain length, and awn development (Jin et al., 2016) and RAE2 (REGULATOR OF AWN ELONGATION 2) which is from African cultivated rice species, O. glaberrima and is involved in awn development (Bessho-Uehara et al., 2016).

According to some embodiments, the gene comprises regulatory regions which control transcription in cis. These are also referred to as a “cis-acting regulatory region” which according to an example is a promoter or an enhancer or a combination of both.

The cis-acting regulatory region is preferably of the OLLS1.

According to a specific embodiment, the promoter region of OLLS1 is as set forth in SEQ ID NO: 10 and 11 (of the OL-IRGC110404 and NIL-107B-12, respectively).

As shown in FIG. 7, the promoter region of OLLS1 comprises deletions and insertions of a few hundreds base pairs and about 20 single nucleotide polymorphisms (SNPs) as compared to other homologs in the family which support a different mode of transcription.

Homologs and orthologs of the gene are provided in SEQ ID NOs: 22-28 (e.g., without the promoter region) and 14-20 (polypeptide sequences).

Without being bound by theory, it is suggested that the promoter region of OLLS1 is unique in that it imparts a spatial expression pattern which is active during stigma development and cell elongation in stigma and is specifically expressed in the pistil and stigma. The promoter region of other gene homologs such as RAE2 and GAD1 does not confer the same spatial expression pattern and hence even though it is expressed in the young panicle, both homologous genes predominantly expressed at awn primordium of lemma in a floret.

According to some embodiments, the expression is done by replacing the promoter to that of OLLS1 (e.g., African rice) and in other embodiments it is done by replacing both the promoter and open reading frame of the OLLS1 homolog (e.g., O. sativa).

According to an aspect there is provided a cultivated Gramineae plant being genetically modified to express a polypeptide encoding OLLS1 as set forth in SEQ ID NO: 12 or 13 or a homolog thereof capable of increasing stigma of the plant as compared to said stigma in a plant of same genetic background and developmental stage as the plant and not subjected to said genetic modification, wherein when said genetic modification is an introgression from Oryza longistaminata encoding said polypeptide, the length of the introgression is shorter than 350 or 300 Kb and comprising a marker selected from the group consisting of ST87, ST89, ST90, ST91, ST92, ST113, ST93 and ST99.

According to an additional or an alternative aspect there is provided a cultivated rice plant comprising an introgression including at least one Oryza longistaminata quantitative trait locus (QTL) associated with stigma length positioned between markers ST87 to ST99 and said introgression being shorter than 350 or 300 Kb.

According to a specific embodiment, the introgression is 250-350 Kb.

According to a specific embodiment, the introgression is shorter than 100 kb, 80 Kb, 50 Kb, 20 Kb, 18 Kb or 10 Kb.

According to a specific embodiment, the introgression is detectable with at least one marker for the QTL associated with stigma length.

According to some embodiments, the marker is selected from the group consisting of ST97, ST87, ST89, ST90, ST91, ST92, ST113, ST93 and ST99.

According to a specific embodiment, the marker is ST92 or ST113.

Specific primers for identification of the markers are provided in Table 5 in the Examples section, which follows (which is to be considered as part of the embodiments of the invention).

According to a specific embodiment, the rice plant comprises at least an additional introgression including at least one Oryza longistaminata QTL associated with stigma area, style length, stigma breadth or total pistil length.

In one particular embodiment, the rice plant comprises at least an additional introgression including at least one Oryza longistaminata QTL associated with stigma area, style length, stigma breadth or total pistil length.

In one particular embodiment, the at least one Oryza longistaminata QTL associated with stigma area, style length, stigma breadth and pistil length is selected from the group consisting of qSTGL2-1, qSTGL5-1, qSTGL11-1, qSTGL11-2; qSTGA8-2; qSTYL1-1, qSTYL5-2, qSTYL8-1; qSTGB1-1, qSTGB3-1; qPSTL1-1, qPSTL1-3 and qPSTL11-1.

In one particular embodiment, a marker set of the at least one additional QTL is selected from the group consisting of stigma area, RM80-RM502 (qSTGA8-2); style length, RM319-RM3640 (qSTYL1-1), RM7653-RM6360 (qSTYL5-2), RM404-RM1109 (qSTYL8-1); stigma breadth, RM4403-RM4319 (qSTGB1-1), RM43525-RM520 (qSTGB3-1); and pistil length, RM3604-RM48134 (qPSTL1-1); RM3640-RM48134 (qPSTL1-3); and RM5997-RM254 (qPSTL11-1).

In one particular embodiment, the rice plant is a line selected from the group consisting of IR68897A, IR68897B, IR58025A, IR58025B, IR127841A, IR127841B IR127842A and IR127842B.

In one particular embodiment, the Gramineae e.g., rice plant is a cytoplasmic male sterile line.

In one particular embodiment, the Gramineae e.g., rice plant is a maintainer line.

In one particular embodiment, the Gramineae e.g., rice plant has an out-crossing rate of at least 60% (or as described herein).

In an aspect of the invention there is provided a cultivated hybrid Gramineae e.g., rice plant having the Gramineae e.g., rice plant having the long stigma, as described herein, as a parent or an ancestor.

In an aspect of the invention there is provided a tissue culture produced from protoplasts or cells from the Gramineae e.g., rice plant having the long stigma, as described herein, wherein the protoplasts or cells of the tissue culture are produced from a plant part selected from the group consisting of: leaves; pollen; embryos; cotyledon; hypocotyls; meristematic cells; roots; root tips; pistils; anthers; flowers; stems; glumes; and panicles.

In an aspect of the invention there is provided a Gramineae plant e.g., rice plant regenerated from the tissue culture, wherein the Gramineae plant e.g., rice plant is a cytoplasmic male sterile Gramineae plant e.g., rice plant having all the morphological and physiological characteristics of the desired rice plant but lacking a functional male reproductive system, e.g., non-viable pollen or pollen which are unable to pollinate the plant (in this case reach the stigma).

In one particular embodiment, a CMS plant of line LST972020A is bred by the methods described herein to comprise the long stigma trait of Oryza longistaminata. The methods make use of at least one marker which is positioned between ST97 or ST87 to ST99. A suitable maintainer line for the converted CMS line LST972020A is line LST972020B.

In another aspect, the present invention provides regenerable cells for use in tissue culture of a CMS plant comprising the long stigma trait of Oryza longistaminata. The tissue culture will preferably be capable of regenerating plants having the physiological and morphological characteristics of the foregoing Gramineae plant e.g., rice plant, and of regenerating plants having substantially the same genotype. Preferably, the regenerable cells in such tissue cultures will be produced from embryo, protoplast, meristematic cell, callus, pollen, leaf, stem, petiole, root, root tip, fruit, seed, flower, anther, pistil or the like. Still further, the present invention provides converted CMS Gramineae plant e.g., rice plants regenerated from tissue cultures of the invention.

Marker Assisted Selection of Converted Maintainer Lines and CMS Lines

In another embodiment described herein, the development of converted maintenance and CMS lines is enhanced by marker assisted selection. Basic protocols for marker assisted selection are well known to one of ordinary skill in the art. Given the benefit of this disclosure, including the quantitative trait loci (QTLs) and markers described herein, one of skill in the art will be able to carry out the invention as described.

A genetic mapping population is generated according to Example 1 of the Examples section which follows. Markers associate with genomic regions controlling stigma length (e.g., QTLs) can then be identified via molecular mapping (see Example 2 and FIG. 1B). These markers are then used to aid in selecting Gramineae plant e.g., rice plants of maintainer or CMS lines successfully introgressed with the long stigma trait of Oryza longistaminata.

Marker-assisted selection (MAS) involves the use of one or more of the molecular markers for the identification and selection of those progeny plants that contain one or more of the genes that encode for the desired trait. In the present instance, such identification and selection is based on the long stigma trait of Oryza longistaminata, and QTLs of the present invention or markers associated therewith. Such are listed in Table 5 but generally they are framed by ST97 or ST87 and ST99. MAS can be used to select progeny plants having the desired trait during the development of the converted maintainer and/or CMS lines by identifying plants harboring the QTL(s) of interest, allowing for timely and accurate selection. Gramineae plant e.g., rice plants developed according to this embodiment can advantageously derive a majority of their traits from the recipient plant (i.e., plant of maintainer or CMS line), and derive the long stigma trait from the donor plant (Oryza longistaminata).

In certain embodiments, one or more markers in progeny plants during the development of converted maintainer lines, converted CMS lines, or both. Detection of one or more markers in a converted line, wherein the marker is linked to a QTL of Oryza longistaminata associated with stigma length and/or total length of stigma and style, is indicative of introgression of the target trait. The QTL can be any one of those QTLs of Table 5 associated with stigma length and/or total length of stigma, area, breadth and style.

According to a specific embodiment, the introgression of the long stigma trait can be detected or is detectable by using markers listed in Table 5, below, e.g., ST97, ST87, ST89, ST90, ST91, ST92, ST113, ST93 and ST99.

According to a specific embodiment, the marker is ST92 or ST113.

The present inventors were able to identify a gene associated with stigma length. The ability to identify the gene of Oryza longistaminata that is associated with the trait now allows for the first time to generate plants of any Gramineae plant using means that are not limited to crossing, but may also include complementation, transgenesis and genome editing.

Thus, according to an embodiment of the invention, expressing in a plant or plant cell the polypeptide is by genome editing of an endogenous nucleic acid sequence encoding the polypeptide or a cis-acting regulatory region of said nucleic acid sequence.

As used herein expressing” or “upregulating” refers to increasing expression at the polypeptide level to an amount exceeding that found in a (control) plant or part thereof (e.g., pistil) of the same genetic background and developmental stage in which said expression has not been attempted.

According to a specific embodiment, upregulating can be by at least 10%, 20%, 30%, 40%, 50%, 60%, 70%, 80%, 90% or even more say, 2 fold, 5 fold, 10 fold, 20 fold 50 fold, 100 fold higher as compared to expression of the corresponding endogenous polypeptide (e.g., SEQ ID NO: 14-20) in the absence of the upregulation treatment.

Thus, according to a specific embodiment expressing is by genome editing of an endogenous nucleic acid sequence encoding said polypeptide or regulatory region of said nucleic acid sequence.

Specifically, genome editing can be used to either reconstitute expression of a correct protein sequence that is able to impart the long stigma trait such as that of Oryza longistaminata (see sequence alignments in FIG. 7).

According to another specific embodiment, genome editing is performed to amend/replace a regulatory sequence within the target plant (e.g., cultivated Gramineae plant e.g., wheat, corm, rice) such as a cis-acting promoter sequence of the relevant genes in the target plant. For example to amend to comprise the regulatory sequence of SEQ ID NO: 10 or 11 or homologs thereof having at least 80%, 85%, 90%, 95%, 99% identity to each of SEQ ID NO: 10 or 11, as long as the modified sequences are able to impart transcription which is in the same spatial pattern and developmental pattern as that of SEQ ID NO: 10 or 11 (e.g., pistil expression).

According to a specific embodiment, the promoter is modified to exclude the sequence marked by green and is absent from the O. longistaminata sequences of FIG. 7.

The skilled artisan will be able to subject the endogenous sequence in the cultivated Gramineae plant to one or more genome editing events or even replacement of the whole gene e.g., regulatory regions of the gene (e.g., to have the genotype of SEQ ID NO: 10 or 11 or a sequence homologous to same as described herein i.e., which confers the pattern of expression of SEQ ID NO: 1 and 2) or only the open reading frame to that of Oryza longistaminata (or a homolog or ortholog thereof) and test the effect on stigma length as described herein, see e.g., Example 2 for the use of genome editing technique.

Following is a non-limiting description of genome editing technologies which can be used to upregulate expression according to some embodiments of the invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDS) and non-homologous end-joining (NHEJF). NHEJF directly joins the DNA ends in a double-stranded break, while HDR utilizes a homologous donor sequence as a template for regenerating the missing DNA sequence at the break point. In order to introduce specific nucleotide modifications to the genomic DNA, a donor DNA repair template containing the desired sequence must be present during HDR.

Genome editing cannot be performed using traditional restriction endonucleases since most restriction enzymes recognize a few base pairs on the DNA as their target and these sequences often will be found in many locations across the genome resulting in multiple cuts which are not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the non-homologous end-joining (NHEJ) pathway often results in small deletions or small sequence insertions. Since each repair made by NHEJ is unique, the use of a single nuclease pair can produce an allelic series with a range of different deletions at the target site.

The deletions typically range anywhere from a few base pairs to a few hundred base pairs in length, but larger deletions have been successfully generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

T-GEE system (TargetGene's Genome Editing Engine)—A programmable nucleoprotein molecular complex containing a polypeptide moiety and a specificity conferring nucleic acid (SCNA) which assembles in-vivo, in a target cell, and is capable of interacting with the predetermined target nucleic acid sequence is provided. The programmable nucleoprotein molecular complex is capable of specifically modifying and/or editing a target site within the target nucleic acid sequence and/or modifying the function of the target nucleic acid sequence. Nucleoprotein composition comprises (a) polynucleotide molecule encoding a chimeric polypeptide and comprising (i) a functional domain capable of modifying the target site, and (ii) a linking domain that is capable of interacting with a specificity conferring nucleic acid, and (b) specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base-pairing of specificity-conferring nucleic acid and a target nucleic acid. The composition is less genotoxic, modular in their assembly, utilize single platform without customization, practical for independent use outside of specialized core-facilities, and has shorter development time frame and reduced costs.

CRISPR-Cas system (also referred to herein as “CRISPR”)—Many bacteria and archaea contain endogenous RNA-based adaptive immune systems that can degrade nucleic acids of invading phages and plasmids. These systems consist of clustered regularly interspaced short palindromic repeat (CRISPR) nucleotide sequences that produce RNA components and CRISPR associated (Cas) genes that encode protein components. The CRISPR RNAs (crRNAs) contain short stretches of homology to the DNA of specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.).

It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded breaks produced by CRISPR/Cas can undergo homologous recombination or NHEJ and are susceptible to specific sequence modification during DNA repair.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs. This creates a system that can be readily modified to target modifications at different genomic sites and/or to target different modifications at the same site. Additionally, protocols have been established which enable simultaneous targeting of multiple genes. The majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH—, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of a gRNA that can be used in the present disclosure include those described in the Example section which follows.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene. Use of clustered regularly interspaced short palindromic repeats (CRISPR)-associated (Cas)-guide RNA technology and a Cas endonuclease for modifying plant genomes are also at least disclosed by Svitashev et al., 2015, Plant Physiology, 169 (2): 931-945; Kumar and Jain, 2015, J Exp Bot 66: 47-57; and in U.S. Patent Application Publication No. 20150082478, which is specifically incorporated herein by reference in its entirety.

This can be done by ‘gene/allele replacement’ to make a long stigma. The donor molecule (OLLSJ promoter or the full-length genomic sequence of OLLSJ containing promoter and CDS) can replace the endogenous alleles in the genome by using genome editing tools. For example—Gene replacements and insertions in rice by intron targeting using CRISPR-Cas9 (Li et al., 2016, Nat Plants 2:16139); Alternatively-Efficient allelic replacement in rice by gene editing: A case study of the NRT1.1B gene (Li et al., 2018, J Integr Plant Biol 60(7):536-540).

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

According to a specific embodiment, the DNA editing agent is CRISPR-Cas9.

According to another specific embodiment, expressing or upregulating is by introducing to the plant a nucleic acid construct comprising a nucleic acid sequence encoding the polypeptide, the nucleic acid sequence being operably linked to a cis-acting regulatory element active in plant cells. Plants generated accordingly are typically transgenic plants.

According to another embodiment, the cis-acting regulatory sequence of the gene is used (e.g., SEQ ID NO: 10 or 11 or homologs thereof as described above). Such as method of expression is also referred to herein as a specific way of transgenesis by complementation e.g., see Example 5 of the Examples section which follows.

Constructs useful in the methods according to some embodiments of the invention may be constructed using recombinant DNA technology well known to persons skilled in the art. The gene constructs may be inserted into vectors, which may be commercially available, suitable for transforming into plants and suitable for expression of the gene of interest in the transformed cells. The genetic construct can be an expression vector wherein said nucleic acid sequence is operably linked to one or more regulatory sequences allowing expression in the plant cells (e.g., SEQ ID NO: 10 or 11 or homologs thereof as described above).

In a particular embodiment of some embodiments of the invention the regulatory sequence is a plant-expressible promoter, heterologous to the gene (e.g., the ORF of O. longistaminata and a heterologous cis-acting regulatory element, e.g., promoter).

As used herein the phrase “plant-expressible” refers to a promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ. Examples of promoters useful for the methods and plants of some embodiments of the invention are described in WOWO2018/224861. These can be constitutively active promoters (e.g., 35S), developmental specific promoters and/or tissue specific promoters (e.g., SEQ ID NO: 10 or 11 or homologs thereof as described herein).

Nucleic acid sequences of the polypeptides of some embodiments of the invention may be optimized for plant expression. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified in order to utilize statistically-preferred or statistically-favored codons within the plant. The nucleotide sequence typically is examined at the DNA level and the coding region optimized for expression in the plant species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1 N [(Xn−Yn)/Yn]2/N, where Xn refers to the frequency of usage of codon n in highly expressed plant genes, where Yn to the frequency of usage of codon n in the gene of interest and N refers to the total number of codons in the gene of interest. A table of codon usage from highly expressed genes of dicotyledonous plants is compiled using the data of Murray et al. (1989, Nuc Acids Res. 17:477-498).

One method of optimizing the nucleic acid sequence in accordance with the preferred codon usage for a particular plant cell type is based on the direct use, without performing any extra statistical calculations, of codon optimization tables such as those provided on-line at the Codon Usage Database through the NIAS (National Institute of Agrobiological Sciences) DNA bank in Japan (www(dot)kazusa(dot)or(dot)jp/codon/). The Codon Usage Database contains codon usage tables for a number of different species, with each codon usage table having been statistically determined based on the data present in Genbank.

By using the above tables to determine the most preferred or most favored codons for each amino acid in a particular species (for example, rice), a naturally-occurring nucleotide sequence encoding a protein of interest can be codon optimized for that particular plant species. This is effected by replacing codons that may have a low statistical incidence in the particular species genome with corresponding codons, in regard to an amino acid, that are statistically more favored. However, one or more less-favored codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively effect mRNA stability or expression.

The naturally-occurring encoding nucleotide sequence may already, in advance of any modification, contain a number of codons that correspond to a statistically-favored codon in a particular plant species. Therefore, codon optimization of the native nucleotide sequence may comprise determining which codons, within the native nucleotide sequence, are not statistically-favored with regards to a particular plant, and modifying these codons in accordance with a codon usage table of the particular plant to produce a codon optimized derivative. A modified nucleotide sequence may be fully or partially optimized for plant codon usage provided that the protein encoded by the modified nucleotide sequence is produced at a level higher than the protein encoded by the corresponding naturally occurring or native gene. Construction of synthetic genes by altering the codon usage is described in for example PCT Patent Application 93/07278.

Thus, some embodiments of the invention encompasses nucleic acid sequences described hereinabove; fragments thereof, sequences hybridizable therewith, sequences homologous thereto, sequences orthologous thereto, sequences encoding similar polypeptides with different codon usage, altered sequences characterized by mutations, such as deletion, insertion or substitution of one or more nucleotides, either naturally occurring or man induced, either randomly or in a targeted fashion.

Plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

As mentioned, according to another embodiment of the invention, expressing or upregulating is by crossing the plant with another plant expressing said polypeptide and selecting for stigma length.

According to a specific embodiment, the method may further comprise determining stigma length of the plant following the upregulating, regardless of the method of expression that is employed.

Methods of determining stigma length are well known in the art and can involve simple measurement with stereomicroscope or a high-resolution scanner.

According to an aspect of the invention there is provided a method of producing a cytoplasmic male sterile Gremineae plant comprising a long stigma trait of Oryza longistaminata, the method comprising crossing the plant of a stable cytoplasmic male sterile line of claim 19 with a rice plant of a suitable maintainer line of claim 20.

According to an aspect of the invention there is provided a method for increasing hybrid seed set in a Gramineae plant comprising:
providing a cytoplasmic male sterile Gramineae plant comprising a long stigma trait of Oryza longistaminata as described herein; and
pollinating the cytoplasmic male sterile plant comprising a long stigma trait of Oryza longistaminata with pollen of a suitable restorer rice line.

According to an aspect of the invention there is provided a method for producing hybrid rice seed comprising:
collecting hybrid seed set on the cytoplasmic male sterile plant comprising the long stigma trait of Oryza longistaminata obtainable according to the methods described herein.

As mentioned, the selection of the long stigma phenotype is done preferably in combination or solely by MAS (also characterization of rice progeny of these methods or products made of such progeny). Thus, also contemplated are primers, probes, amplicons and/or kits comprising same which can be diagnostic of the introgression of the invention (long stigma from Oryza longistaminata).

The nucleic acid probes and primers of the present invention hybridize under stringent conditions to a target DNA sequence. Any conventional nucleic acid hybridization or amplification method can be used to identify the presence the long stigma introgression from Oryza longistaminata in a sample. Nucleic acid molecules or fragments thereof are capable of specifically hybridizing to other nucleic acid molecules under certain circumstances. As used herein, two nucleic acid molecules are capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., 1989, and by Haymes et al., In: Nucleic Acid Hybridization, A Practical Approach, IRL Press, Washington, D.C. (1985), Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

Regarding the amplification of a target nucleic acid sequence (e.g., by PCR) using a particular amplification primer pair, “stringent conditions” are conditions that permit the primer pair to hybridize only to the target nucleic-acid sequence to which a primer having the corresponding wild-type sequence (or its complement) would bind and preferably to produce a unique amplification product, the amplicon, in a DNA thermal amplification reaction.

For example, to determine whether the rice plant resulting from a sexual cross contains the long stigma introgression from Oryza longistaminata from the rice plant of the present invention, DNA extracted from a rice plant tissue sample (e.g., endosperm of a seed/meal/grain of a rice plant having long stigma as described herein e.g., of a hybrid plant) may be subjected to nucleic acid amplification method using a primer pair that includes a primer derived from flanking sequence in the genome of the plant adjacent to the insertion site of inserted heterologous DNA, and a second primer derived from the inserted heterologous DNA to produce an amplicon that is diagnostic for the presence of the long stigma introgression from Oryza longistaminata. The amplicon is of a length and has a sequence that is also diagnostic for the long stigma introgression from Oryza longistaminata. The amplicon may range in length from the combined length of the primer pairs plus one nucleotide base pair, preferably plus about fifty nucleotide base pairs, more preferably plus about two hundred-fifty nucleotide base pairs, and even more preferably plus about four hundred-fifty nucleotide base pairs. Alternatively, a primer pair can be derived from flanking sequence on both sides of the inserted DNA so as to produce an amplicon that includes the entire insert nucleotide sequence. A member of a primer pair derived from the plant genomic sequence may be located a distance from the inserted DNA molecule, this distance can range from one nucleotide base pair up to about twenty thousand nucleotide base pairs. The use of the term “amplicon” specifically excludes primer dimers that may be formed in the DNA thermal amplification reaction.

Nucleic-acid amplification can be accomplished by any of the various nucleic-acid amplification methods known in the art, including the polymerase chain reaction (PCR). A variety of amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. PCR amplification methods have been developed to amplify up to 22 kb of genomic DNA and up to 42 kb of bacteriophage DNA (Cheng et al., Proc. Natl. Acad. Sci. USA 91:5695-5699, 1994). These methods as well as other methods known in the art of DNA amplification may be used in the practice of the present invention. The sequence of the introgression or flanking sequence can be verified (and corrected if necessary) by amplifying such sequences from the long stigma introgression from Oryza longistaminata using primers derived from the sequences provided herein followed by standard DNA sequencing of the PCR amplicon or of the cloned DNA.

The amplicon produced by these methods may be detected by a plurality of techniques. One such method is Genetic Bit Analysis (Nikiforov, et al. Nucleic Acid Res. 22:4167-4175, 1994) where a DNA oligonucleotide is designed which overlaps both the adjacent flanking genomic DNA sequence and the inserted DNA sequence. The oligonucleotide is immobilized in wells of a microwell plate. Following PCR of the region of interest (using one primer in the inserted sequence and one in the adjacent flanking genomic sequence), a single-stranded PCR product can be hybridized to the immobilized oligonucleotide and serve as a template for a single base extension reaction using a DNA polymerase and labeled ddNTPs specific for the expected next base. Readout may be fluorescent or ELISA-based. A signal indicates presence of the insert/flanking sequence due to successful amplification, hybridization, and single base extension.

Another method is the pyrosequencing technique as described by Winge (Innov. Pharma. Tech. 00:18-24, 2000). In this method an oligonucleotide is designed that overlaps the adjacent genomic DNA and insert DNA junction. The oligonucleotide is hybridized to single-stranded PCR product from the region of interest (one primer in the inserted sequence and one in the flanking genomic sequence) and incubated in the presence of a DNA polymerase, ATP, sulfurylase, luciferase, apyrase, adenosine 5′ phosphosulfate and luciferin. dNTP's are added individually and the incorporation results in a light signal which is measured. A light signal indicates the presence of the long stigma introgression from Oryza longistaminata due to successful amplification, hybridization, and single or multi-base extension.

Fluorescence polarization as described by Chen, et al., (Genome Res. 9:492-498, 1999) is a method that can be used to detect the amplicon of the present invention. Using this method an oligonucleotide is designed which overlaps the genomic flanking and inserted DNA junction. The oligonucleotide is hybridized to single-stranded PCR product from the region of interest (one primer in the inserted DNA and one in the flanking genomic DNA sequence) and incubated in the presence of a DNA polymerase and a fluorescent-labeled ddNTP. Single base extension results in incorporation of the ddNTP. Incorporation can be measured as a change in polarization using a fluorimeter. A change in polarization indicates the presence of the long stigma introgression from Oryza longistaminata due to successful amplification, hybridization, and single base extension.

Taqman®. (PE Applied Biosystems, Foster City, Calif) is described as a method of detecting and quantifying the presence of a DNA sequence and is fully understood in the instructions provided by the manufacturer. Briefly, a FRET oligonucleotide probe is designed which overlaps the genomic flanking and insert DNA junction. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the long stigma introgression from Oryza longistaminata due to successful amplification and hybridization.

Molecular Beacons have been described for use in sequence detection as described in Tyangi, et al. (Nature Biotech. 14:303-308, 1996) Briefly, a FRET oligonucleotide probe is designed that overlaps the flanking genomic and insert DNA junction. The unique structure of the FRET probe results in it containing secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe to the target sequence results in the removal of the probe secondary structure and spatial separation of the fluorescent and quenching moieties that results in the production of a fluorescent signal. The fluorescent signal indicates the presence of the long stigma introgression from Oryza longistaminata due to successful amplification and hybridization.

Other described methods, such as, microfluidics (US Patent pub. 2006068398, U.S. Pat. No. 6,544,734) provide methods and devices to separate and amplify DNA samples. Optical dyes used to detect and quantitate specific DNA molecules (WO/05017181). Nanotube devices (WO/06024023) that comprise an electronic sensor for the detection of DNA molecules or nanobeads that bind specific DNA molecules and can then be detected.

DNA detection kits are provided using the compositions disclosed herein. The kits are useful for the identification of the long stigma introgression from Oryza longistaminata in a sample and can be applied at least to methods for breeding rice plants containing the appropriate introgressed DNA. The kits contain DNA primers and/or probes that are homologous or complementary to segments i.e., markers which are listed in Table 5 and specifically, those positioned between ST97 or ST87 and ST99. Primers for these sequences are listed in Table 5 and can be used in DNA amplification reactions or as probes in a DNA hybridization method for detecting the presence of polynucleotides diagnostic for the presence of the target DNA in a sample. The production of a predefined amplicon in a thermal amplification reaction is diagnostic for the presence of DNA corresponding to the long stigma introgression from Oryza longistaminata in the sample. If hybridization is selected, detecting hybridization of the probe to the biological sample is diagnostic for the presence of the long stigma introgression from Oryza longistaminata in the sample. Typically, the sample is rice, or rice products or by-products of the use of rice.

Also provided are processed rice products which are produced from the plants described herein and preferably contain the nucleic acid sequence conferring the improved out-crossing rate described herein. Also provided are methods of processing the rice (e.g., to produce meal) or other processed products.

Thus, for example, according to an aspect of the invention there is provided a method of producing meal, the method comprising:
(a) growing and collecting seeds of the hybrid plant as described herein; and
(b) processing said seeds to meal.

Food Characteristics:

Rice starch is a major source of carbohydrate in the human diet, particularly in Asia, and the grain of the invention and products derived from it can be used to prepare food. The food may be consumed by man or animals, for example in livestock production or in pet-food. The grain derived from the rice plant can readily be used in food processing procedures, and therefore the invention includes milled, ground, kibbled, cracked, rolled, boiled or parboiled grain, or products obtained from the processed or whole grain of the rice plant, including flour, brokers, rice bran and oil. The products may be precooked or quick-cooking rice, instant rice, granulated rice, gelatinized rice, canned rice or rice pudding. The grain or starch may be used in the production of processed rice products including noodles, rice cakes, rice paper or egg roll wrapper, or in fermented products such as fermented noodle or beverages such as sake. The grain or starch derived therefrom may also be used in, for example, breads, cakes, crackers, biscuits and the like, including where the rice flour is mixed with wheat or other flours, or food additives such as thickeners or binding agents, or to make drinks, noodles, pasta or quick soups. The rice products may be suitable for use in wheat-free diets. The grain or products derived from the grain of the invention may be used in breakfast cereals such as puffed rice, rice flakes or as extruded products.

Dietary Fiber:

Dietary fiber, in this specification, is the carbohydrate and carbohydrate digestion products that are not absorbed in the small intestine of healthy humans but enter the large bowel.

This includes resistant starch and other soluble and insoluble carbohydrate polymers. It is intended to comprise that portion of carbohydrates that are fermentable, at least partially, in the large bowel by the resident microflora.

Non-Food Applications:

Rice is widely used in non-food industries, including the film, paper, textile, corrugating and adhesive industries, for example as a sizing agent. Rice starch may be used as a substrate for the production of glucose syrups or for ethanol production.

Similar processed products are present for other Gramineae species.

Thus, there is provided any of the following products or uses, which constitute a non-limiting list. Wheat or maize flour, starch, gluten, meal and products thereof (e.g., bread), flour for leavened, flat and steamed breads, biscuits, cookies, cakes, breakfast cereal, pasta, noodles, couscous, fermentation to make beer, alcoholic beverages, biofuel, silage, building materials, canners/packers, chemicals. Condiments, confectionary, fats and oils, formulated dairy products, fuel alcohol, household needs, ice creams, frozen desserts, jams, jellies preserves, paper and related products, syrups and sweeteners, textile (clothing, carpeting, bedding).

The present invention also contemplates methods of producing the processed product or product.

For example there is provided a method of producing wheat or maize meal, the method comprising:
(a) harvesting grains of the plant of the invention; and
(b) processing the grains to produce the wheat meal.

Alternatively, there is provided a method of producing oil, the method comprising:
(a) harvesting grains of the plant of the invention; and
(b) extracting oil from the grains.

Also provided is the use of the seeds in oil and meal production.

Additionally or alternatively, there is provided a method of producing dry matter, the method comprises harvesting the dry matter of the plant which comprises the SV, as described herein and optionally further processing the dry matter. Generally, the dry matter comprises the leaves, husk, head, tillers and stem of wheat, left in the field after harvest or artificially dried.

DNA detection in the processed products can be performed using methods which are well known in the art and are described in some detail hereinabove.

Thus, the markers can be to any of the loci (e.g., ST97 or ST87 to ST99 and any marker inbetween) described herein which are associated with high out-cross rate.

It is expected that during the life of a patent maturing from this application many relevant markers will be developed and the scope of the term marker is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Plant Materials

Three different NILs were used which comprise the qSTGL8.0, specifically, NIL_6i14-191 from the IR64×OL (IRGC110404) cross, NIL_91B-42 from the IR58025B×OL (IRGC110404) cross, and NIL_107B-12 from the IR68879B×OL (IRGC92664) cross, developed by IRRI. The three recurrent background varieties, IR64, IR58025B, and IR68879B were used for backcross. NIL_6i14-191 and IR64 were used as a background material of rice transformation for CRISPR-Cas9 tool-based knock-out (KO) and complementation test, respectively.

DNA Preparation, PCR Genotyping, and Marker Development for Fine Mapping

About 500 seeds derived from each backcrossed F₁ plant were seeded. After two weeks, leaf samples were collected into 2 mL tubes containing two steel balls. Genomic DNA was prepared by using a modified simple DNA preparation method (Kim et al., 2016) which does not require phenol/chloroform extraction and isopropanol precipitation steps. Briefly, 500 μL of TPE buffer (100 mM Tris-HCl pH 9.5, 1 M KCl, 10 mM EDTA pH 8.0) was added to the 2 mL tube containing leaf tissue and two steel balls and the samples were homogenized using a 2010 Geno/Grinder (SPEX: www(dot)spexsampleprep(dot)com) without liquid nitrogen, and then the sample tubes were incubated at 65° C. for 30 min. After vigorous shaking by hands, the sample tubes were centrifuged at the maximum speed for 10 min. The supernatant was transferred into 96-well plate containing ddH₂O for dilution (supernatant:water=1:5 ratio) and the crude extracts was used as template DNA for PCR genotyping. PCR genotyping followed normal PCR conditions (annealing at 55° C., extension at 72° C. for 60s, 35 cycles) and the PCR products were analyzed in 3% agarose gel. While narrowing down the qSTGL8.0, the required InDel type markers were identified for the specific region using the public sequence information and our whole genome sequencing data of the NIL_107B-12.

Phenotyping

For stigma phenotyping, 10 opening spikelets per plant were collected and placed on wet paper towel in Petridis in the morning (09:00-12:OOAM). Spikelets were dissected under stereo microscope and stigmas were placed on a slide glass (five stigmas/plant×5 plants on one slide glass). The stigma images were obtained by using a scanner (Epson Perfection V700 Photo). The images of spikeltes and grains were also prepared in s similar manner. For phenotyping the major agronomic traits, more than 100 F₂ plants per population were genotyped by using the ST05 marker and 8-10 homozygous plants for each allele (Re/Re and OL/OL) were randomly selected and phenotyped. Mean value of each trait from each homozygous group was obtained and compared by Student's t-test.

Vector Construction and Rice Transformation

For knock-out of the candidate genes the pSR339 binary vector containing pUbi1-SpCas9-tNos::pOsU3-LacZ-sgRNA::p35S-HPT-t35S cassettes on the T-DNA region was used. The CRISPR-Cas9 target site (20-bp guide sequence) of each candidate gene was screened by using the RGEN Tools (www(dot)rgenome(dot)net/) and selected at the common sequence between IR64 and OL (IRGC110404). The 20-bp dsDNA molecules were prepared by the duplexed oligomers: 5-GGCAGCTCAAGGCGCAGCAGTGGG-3 (SEQ ID NO: 116) and 5-AAACCCCACTGCTGCGCCTTGAGC-3 (SEQ ID NO: 117) for Os08g37810, 5-GGCACACCACGGCCAGCTGCTCAC-3 (SEQ ID NO: 118) and 5-AAACGTGAGCAGCTGGCCGTGGTG-3 (SEQ ID NO: 119) for Os08g37840, 5-GGCAGGCGAGCAGCAACGCAGAG-3 (SEQ ID NO: 120) and 5-AAACCTCTGCGTTGCTGCTCGCC-3 (SEQ ID NO: 121) for Os08g37890 (20 nt-transcribed guide sequence by OsU3 promoter is underlined) to make KO of each corresponding gene of OL in the NIL_91B-42. Finally, the duplex DNA molecule was replaced with LacZ of pSR339 vector by type II AarI restriction enzyme digestion of pSR339, ligation with the duplex oligomer, and blue/white colony selection processes. Each CRISPR-Cas9 construct was named as pIRS1492 for Os08g37810, pIRS1493 for Os08g37890, and pIRS1494 for Os08g37840, respectively. The above constructs were transferred into Agrobacterium tumefaciens (LBA4404 strain). Immature segregating F₂ seeds from the backcrossed plants (IR64×NIL_6i14-191) were transformed by Agrobacterium transformation method for indica rice variety (Slamet-Loedin et al., 2014). For complementation test, ˜4.4 kb genomic segment of OLLS1 was cloned from NIL_107B-12 into PCR-subcloning plasmid by using the 37890-comp-F1/-R4 primer set and high fidelity Pfu DNA polymerase (BIOFACT: www(dot)bio-ft(dot)com/). The PCR-subcloned plasmid was sequenced by Macrogen (www(dot)macrogen(dot)com/) and finally the ˜4.4 kb fragment was transferred into binary vector, pSR360. The construct was transformed with IR64 variety by using the above Agrobacterium method. More than 120 independent primary T₀ transgenic plants were obtained for each construct. As control plants, a tissue culture (without Agrobacterium co-cultivation) derived plants from both transformation background materials (NIL_6i14-191 and IR64). All the transgenic plants and corresponding control plants were grown at CL4 of the IRRI transgenic facility.

Sequencing of CRISPR-Cas9 Target Sites

Firstly the genotypes of qSTGL8.0 (IR64/IR64, IR64/OL, and OL/OL) were identified from all the primary transgenic plants using the ST109 marker because the CRISPR-Cas9 construct was transformed to immature segregating F₂ seeds. To check the target sequences editing, the OL/OL homozygous plants were selected and the PCRs were performed with each primer set (37810-TS-seq-F1B/-R2 for Os08g37810, 37840-TS-seq-F2/-R4 for Os08g37840, and 37890-TS-seq-F2/-R1 for Os08g37890) for amplification of the CRISPR-Cas9 target site of each corresponding OL gene. The PCR products were directly sequenced by using Applied Biosystems AB3730 DNA analyzer at Macrogen. The sequencing results were opened using Chromas software and the edited sequences were manually analyzed. For sequence analysis of Os08g37890 target region from the heterozygote (IR64/OL), allele specific amplification was performed with 37890-a-NB-F1/-R1 for IR64 allele and 37890-a-404-F1/-R1 for OL (IRGC110404) allele, respectively and the PCR products were sequenced separately. The primers used for molecular analysis are presented in Table 1.

TABLE 1
The primers used for molecular analysis in this study

| Primer name | Sequence (5-3)/SEQ ID NO: | Purpose |
|---|---|---|
| 37810-TS-seq- | CACCCGAATCCGCCTCCACCA/137 | PCR & sequencing of the CRISPR-Cas9 target |
| F1B | | (Os08g37810 homolog) |
| 37810-TS-seq- | AAGGCGGAGGAGACAGGGAGC/138 | PCR & sequencing of the CRISPR-Cas9 target |
| R2 | | (Os08g37810 homolog) |
| 37840-TS-seq- | GAGAGCGACGCGTCGAGCACCT/139 | PCR & sequencing of the CRISPR-Cas9 target |
| F2 | | (Os08g37840 homolog) |
| 37840-TS-seq- | GCCCACTGGACCAAGCTCACC/140 | PCR & sequencing of the CRISPR-Cas9 target |
| R4 | | (Os08g37840 homolog) |
| 37890-TS-seq- | CTCAGTGCTGCTCACTGCCTCACT/141 | PCR & sequencing of the CRISPR-Cas9 target |
| F2 | | (Os08g37890 homolog) |
| 37890-TS-seq- | CCACCTCGCCACCAACCTGCATC/142 | PCR & sequencing of the CRISPR-Cas9 target |
| R1 | | (Os08g37890 homolog) |
| 37890-a-NB-F1 | CGTCTTGCTGCTTCCAGTC/143 | OsEPFL1-IR64 allele specific PCR |
| 37890-a-NB-R1 | GCATCAACTAACCGAACAAAATTT/144 | OsEPFL1-IR64 allele specific PCR |
| 37890-a-404-F1 | ATCTCACAGCCCCCAGTC/145 | OLLS1-OL(IRGC 110404) allele specific PCR |
| 37890-a-404-R1 | GCCATGGACGCACACAGCA/146 | OLLS1-OL(IRGC 110404) allele specific PCR |
| 37890-comp-F1 | gaagctTGCCAATATCTCTTGCCTCTTGGAAG/147 | Complementation test vector construction |
| 37890-comp-R4 | tgaatTCCCTGGCATGAAACCTCAAATGAAC/148 | Complementation test vector construction |
| 37890-F2 | TGGAATCGTTGTCGTCGCGT/149 | qRT-PCR of OsEPFLI/OLLSI |
| 37890-R2 | GAGCAGATGCCAGTGAAAGA/150 | qRT-PCR of OsEPFLI/OLLSI |
| HPH-979-F | TGCTCCGCATTGGTCTTGAC/151 | T-DNA detection through HPT gene PCR |
| tCaMV-R | GGAGAAACTCGAGCTTGTCGA/152 | T-DNA detection through HPT gene PCR |

The underlined sequences are the Hind III and Eco RI restriction sites respectively for cloning of the 4.4 kb OLLSI into binary vector pSR360.

Whole Genome Sequencing, De Novo Assembly, and Sequence Analysis

Genomic DNA was prepared from leaf tissue of the NIL_107B-12 possessing the qSTGL8.0-OL (IRGC92664) by the modified CTAB method. Whole genome sequencing was done by using Illumina HiSeq X-10 platform (350 bp insert library without PCR and 150 bp PE) through Macrogen and produced 58.6 Gb yield (˜154× of the reference genome). All the raw reads were imported into the CLC Genomics Workbench software (www(dot)qiagen(dot)com/) and were processed as follows: 15-bp trimming out of each 150 bp read (5′-5 bp and 3′-10 bp) to remove less accurate sequencing region and then de novo sequence assembly (word size: 64, minimum contig size: 1500 bp). To isolate the contigs covering the qSTGL8.0-OL, BLAST was conducted by using the 563 kb of the reference sequence (23.6-24.2 Mb) as a bait sequence with the database consisting of the above de novo assembly-derived contigs (18,669 contigs).

Finally, 14 contigs were isolated and manually assembled, resulting in 484 kb length of OL. The corresponding sequences of 563 kb bait sequence from IR8, IR64, Minghui63, and another accession of O. longistaminata (IRGC110404) were obtained from public databases. The above sequences were multiple-aligned by using a web-based VISTA tool (www(dot)genome(dot)lbl(dot)gov/vista/customAlignment. shtml) (Dubchak and Ryaboy 2006). The multiple sequence alignments data was used for DNA marker development and candidate gene selection.

Real-Time PCR Analysis

For RNA sample preparations, two NILs (NIL_6i14-191 and NIL-107B-12) and their corresponding backgrounds (IR64 and IR68897B) were seeded. Leaf and root tissues were collected from 8 days-old seedlings which were grown on ½ strength MS media. Developing panicles (1-2 cm, 4 cm, 10 cm in total length), spikelet at the spikelet opening time, pistil including stigma from the opening spikelet, and developing seeds (5 days after pollination) were collected from the plants grown in the paddy field. All the samples (three biological replications for each sample) were immediately frozen in liquid nitrogen and they were stored at −80° C. till all the samples are ready. Total RNA was extracted by using PureLink Plant RNA Reagent (ThermoFisher: www(dot) thermofisher(dot)com/) and cDNA was synthesized using an ImProm-II Reverse Transcription system (Promega: www(dot)worldwide(dot)promega(dot)com/). qRT-PCRs was conducted by using 37890-F2/R2 primers (Table 1) annealed to OsEPFL1/OLLS1 and the SYBR select master mix (ThermoFisher) in ABI7500 machine (ThermoFisher). The OsAct1 gene was used as an internal control and the relative expression level was calculated based on the ΔΔCt method.

Precision Marker-Based Breeding

To minimize presence of unwanted traits in the final breeding products caused by linkage drag and/or the target gene-unlinked donor introgressions, precision marker-based breeding was performed. The long-exserted stigma NIL in the commercial hybrid parental background IR68897B (Line: NIL_107B-12) was backcrossed with IR68897B, resulted in heterozygous at OLLS1 locus. In the following F₂ generation, we selected recombinants near the OLLS1 by genotyping with OLLS1 flanking markers (Table 5) to reduce linkage drag. After trimming of one side of the OLLS1-introgression, another round of recombinant selection was performed from the progenies of the selected recombinants to trim another side of the introgression. Finally we selected three lines which possessed the smallest sizes of OLLS1-introgression (230˜350 kb) and the lines were backcrossed again to increase the genome of recurrent. Then, three selected breeding lines were crossed with A line (IR68897A) to transfer the small OLLS1-containing segment to A line. In the following generation, OLLSJ heterozygous plants were selected by ST89 marker and the plants were crossed with the OLLSJ homozygous B line to obtain homozygous OLLSJ A lines. Eventually, we obtained three homozygous lines possessing the 230˜350 kb sizes of OLLSJ introgression in the IR68897B/A backgrounds (Selected lines: LST972020-VIP02, LST972020-VIP34, LST972020-VIP12).

### Example 1

### Long Stigma Phenotype Inherited by a Single Dominant Allele

In a previous study, the long stigma QTL, the qSTGL8.0 derived from the two different O. longistaminata (OL) accessions (IRGC110404 and IRGC92664) were successfully transferred to two sets of commercial hybrid parental lines IR58025A (A: cytoplasmic male sterile line)/IR58025B (B: maintainer line) and IR68897A/IR68897B respectively and the two sets of near-isogenic lines (NILs) possessing qSTGL8.0 exhibited long stigma and showed significantly higher out-crossing rate compared to those of the original AB combinations (Jena et al., 2016). To reveal the inheritance pattern of both the genotype (qSTGL8.0) and phenotype (long stigma), the newly developed B lines, the NIL_91B-42 possessing the qSTGL8.0-OL (IRGC110404) and the NIL_107B-12 possessing the qSTGL8.0-OL (IRGC92664) were crossed with their corresponding recurrent (Re), IR58025B and IR68897B, respectively and the segregation patterns were observed in the following generation F₂s. Four F₁ (IR58025B×NIL_91B-42) and seven F₁ (IR68897B×NIL_107B-12) plants in each background were selected and self-pollinated to produce F₂ plants. More than 300 F₂ plants from each F₁ plant were genotyped by using the PA08-62 marker located within the qSTGL8.0 (FIG. 1A) and the chi-square (χ²) test was applied to determine the allele segregation pattern at qSTGL8.0 locus. Out of 11 F₂ populations, nine showed a normal Mendelian segregation pattern (Re/Re:Re/OL:OL/OL=1:2:1 ratio) of qSTGL8.0 (Table 2), suggesting that the OL allele of qSTGL8.0 did not affect normal development of male and female gametophytes as well as zygotic embryo.

TABLE 2

| F₁ (female × scale) | | F₂ genotypes^{a} | | | Total F₂ | | df = 2, |
|---|---|---|---|---|---|---|---|
| plant number | | Re/Re | Re/OL | OL/OL | plant no. | x² | α = 0.05 |
| F₁ (IR58025B × NIL_91B-42)-#2 | Observed number (O) | 62 | 175 | 102 | 339 | | |
| | Expected number (E) | 84.75 | 169.50 | 84.75 | 339 | | |
| | (O-E) [symbol] | 6.11 | 0.18 | 3.51 | | 9.80 [symbol] | 5.99 |
| F₁ (IR58025B × NIL_91B-42)-#7 | Observed number (O) | 90 | 193 | 100 | 383 | | |
| | Expected number (E) | 95.75 | 191.50 | 95.75 | 383 | | |
| | (O-E) [symbol] | 0.35 | 0.01 | 0.19 | | 0.55 | 5.99 |
| F₁ (IR58025B × NIL_91B-42)-#8 | Observed number (O) | 79 | 192 | 110 | 381 | | |
| | Expected number (E) | 95.25 | 190.50 | 95.25 | 381 | | |
| | (O-E) [symbol] | 2.77 | 0.01 | 2.28 | | 5.07 | 5.99 |
| F₁ (IR58025B × NIL_91B-42)-#10 | Observed number (O) | 94 | 189 | 100 | 383 | | |
| | Expected number (E) | 95.75 | 191.50 | 95.75 | 383 | | |
| | (O-E) [symbol] | 0.03 | 0.03 | 0.19 | | 0.25 | 5.99 |
| F₁ (IR68897B × NIL_107B-12)- [symbol] | Observed number (O) | 100 | 185 | 87 | 373 | | |
| | Expected number (E) | 93.00 | 186.00 | 93.00 | 372 | | |
| | (O-E) [symbol] | 0.53 | 0.01 | 0.39 | | 0.92 | 5.99 |
| F₁ (IR68897B × NIL_107B-12)- [symbol] | Observed number (O) | 88 | 177 | 113 | 378 | | |
| | Expected number (E) | 94.50 | 189.00 | 94.50 | 378 | | |
| | (O-E) [symbol] | 0.45 | 0.76 | 3.62 | | 4.83 | 5.99 |
| F₁ (IR68897B × NIL_107B-12)- [symbol] | Observed number (O) | 91 | 194 | 94 | 381 | | |
| | Expected number (E) | 95.25 | 190.50 | 95.25 | 381 | | |
| | (O-E) [symbol] | 0.05 | 0.06 | 0.02 | | 0.13 | 5.99 |
| F₁ (IR68897B × NIL_107B-12)-#4 | Observed number (O) | 101 | 193 | 90 | 354 | | |
| | Expected number (E) | 96.00 | 192.00 | 96.00 | 354 | | |
| | (O-E) [symbol] | 0.26 | 0.01 | 0.38 | | 0.64 | 5.99 |
| F₁ (IR68897B × NIL_107B-12)-#6 | Observed number (O) | 99 | 156 | 95 | 353 | | |
| | Expected number (E) | 95.75 | 191.50 | 95.75 | 353 | | |
| | (O-E) [symbol] | 0.11 | 0.16 | 0.05 | | 0.32 | 5.99 |
| F₁ (IR68897B × NIL_107B-12)-#8 | Observed number (O) | 94 | 195 | 95 | 384 | | |
| | Expected number (E) | 96.00 | 192.00 | 96.00 | 384 | | |
| | (O-E) [symbol] | 0.04 | 0.05 | 0.01 | | 0.10 | 5.99 |
| F₁ (IR688978 × NIL_107B-12)-#9 | Observed number (O) | 75 | 208 | 82 | 365 | | |
| | Expected number (E) | 91.25 | 182.50 | 91.25 | 365 | | |
| | (O-E) [symbol] | 2.89 | 3.56 | 0.94 | | 7.39 [symbol] | 5.99 |

^{a}Genotypes were defined by the PA08-62 marker. Re: recurrent allele (IR58025B or IR68897B), OL: O. longistaminata allele (IRGC92664 or IRGC110404) present in the NILs.
*Except for the two F₂s populations derived from the F₁ (IR58025B × NIL_91B-42)-#2 and F₁ (IR68897B × NIL_107B-12)-#9, all F₂s showed the normal Mendelian segregation pattern (Re/Re:Re/OL:OL/OL = 1:2:1 ratio). [symbol]
indicates data missing or illegible when filed

For analysis of phenotype segregation patterns, about 100 F2 plants derived from one F₁ plant in each background (IR58025B and IR68897B) were randomly selected and stigma phenotyped. The segregation ratio of long:short stigma was around 3:1 in both F₂ populations based on the chi-square (χ²) test (Table 3), indicating that the long stigma phenotype is dominant trait and it is defined by a single dominant allele.

TABLE 3
Inheritance patterns of stigma phenotype in segregating F₂ progenies derived from the F₁ (recurrent × NIL-OL)

| F₁ (female × male) | | F₂ stigma phenotype^{a} | | Total F₂ | | df = 1, |
|---|---|---|---|---|---|---|
| plant number | | Short | Long | plant no. | χ² | α = 0.05 |
| F₁ (IR68897B × NIL_107B-12)-#9 | Observed number (O) | 23 | 81 | 104 | | |
| | Expected number (E) | 26 | 78 | 104 | | |
| | (O-E)²/E | 0.35 | 0.12 | | 0.46 | 3.84 |
| F₁ (IR58025B × NIL_91B-42)-#8 | Observed number (O) | 30 | 112 | 142 | | |
| | Expected number (E) | 35.5 | 106.5 | 142 | | |
| | (O-E)²/E | 0.85 | 0.28 | | 1.14 | 3.84 |

^{a}Five stigmas were collected from each F₂ plant and were phenotyped.

Furthermore, the above phenotyped 246 F2 plants were genotyped by using the ST05 marker linked to qSTGL8.0. The result showed that all long stigma plants had heterozygous (Re/OL) or homozygous (OL/OL) genotypes while all the short stigma plants comprised the homozygous recurrent alleles (Re/Re). These data concluded that the long stigma phenotype is governed by the single dominant qSTGL8.0 allele derived from OL.

In order to examine the genetic effect of qSTGL8.0-OL allele on major agronomic traits, several agronomic traits were collected including: plant height, tiller number, panicle length, panicle branching number, grain number per panicle, and spikelet fertility. Mean values of each trait were compared between two homozygote groups (Re/Re and OL/OL) from the four different segregating F2 populations. However, there was no consistent significant difference between two alleles for the all traits measured (Table 4), suggesting that the qSTGL8.0 is not associated with the traits tested except for the stigma length.

TABLE 4
Comparison of major agronomic traits between two genotypes (Re/Re
and OL/OL) at qSTGL8.0 from four different F₂ populations

| | Genotype of | | | | | | | |
|---|---|---|---|---|---|---|---|---|
| F₁ (female × male) | qSTGL8.0 | PH | | PL | | | | SF |
| plant number | in F₂ | (cm) | TN | (cm) | PBN | SBN | GNPP | (%) |
| F₁ (IR58025B × NIL_91B-42)-#8 | Re/Re (IR58025B) | 89.85 | 13.00 | 25.17 | 11.17 | 44.70 | 190.23 | 83.65 |
| | OL/OL (IRGC110404) | 89.75 | 11.70 | 23.68** | 12.73** | 47.60 | 200.17 | 82.85 |
| F₁ (IR58025B × NIL_91B-42)-#11 | Re/Re (IR58025B) | 98.88 | 14.63 | 27.58 | 12.88 | 61.71 | 273.21 | 80.20 |
| | OL/OL (IRGC110404) | 94.95 | 10.00* | 23.67** | 13.50 | 57.60 | 230.30 | 80.62 |
| F₁ (IR68897B × NIL_107B-12)-#7 | Re/Re (IR68897B) | 81.80 | 14.70 | 24.05 | 7.83 | 30.90 | 157.03 | 81.32 |
| | OL/OL (IRGC92664) | 79.33 | 13.33 | 22.94 | 8.04 | 29.04 | 144.48 | 85.76 |
| F₁ (IR68897B × NIL_107B-12)-#10 | Re/Re (IR68897B) | 92.45 | 17.90 | 25.03 | 9.30 | 36.00 | 186.33 | 78.22 |
| | OL/OL (IRGC92664) | 90.95 | 13.30* | 23.37 | 9.73 | 34.50 | 175.20 | 85.87** |

Asterisks represent significant difference between two alleles based on Student's t-test (*α = 0.05 and **α = 0.01). (n = 8 to 10 plants). Re: recurrent allele, OL: OL allele, PH: plant height, TN: tiller number, PL: panicle length, PBN: primary branching number of panicle, SBN: secondary branching number of panicle, GNPP: grain number per panicle, SF: spikelet fertility = (filled GN/total GN) × 100

### Example 2

### Fine Mapping of qSTGL8.0 Narrowed Down to an about 142 kb Region on Chromosome 8

The genetic locus of qSTGL8.0 was defined by two border markers, RM7356 and RM256 (˜3.0 Mb size), on chromosome 8 by using the mapping populations derived from the IR64×OL (IRGC110404) cross (WO2018/224861). Through an additional mapping of the same populations, the locus was further narrowed down to be bordered by ST69 and RM256 markers which is about 683 kb size on the rice reference genome (FIG. 1A). To expect an increased recombination rate at the qSTGL8.0 locus, two additional F2 populations were used for the above genotype/phenotype segregation analysis. In total 4,179 F2 plants were genotyped with four markers (ST69, PA08-62, ST05, and ST54) and the result located a ˜316 kb region defined by PA08-62 and ST05 markers (FIG. 1A). To dissect the ˜316 kb region, 3,120 F3 plants were additionally genotyped. About 30 markers were developed (Table 5) to locate the precise recombination site from the selected recombinant plants.

TABLE 5
The markers used for fine mapping

| Marker | Marker location | Forward primer (5′→3′)/ | Reverse primer (5′→3′)/ |
|---|---|---|---|
| name | (bp)* | SEQ ID NO: 30-64 | SEQ ID NO: 65-99 |
| RM7356 | 21,282,849 | CCAAGGACACATATGCATGC | GCAATTCATGGCGCTGTTC |
| PA08-05 | 21,363,486 | AATTGTTCCGGTGGACTCAT | TTAGAATGCACCCCATGTTCT |
| PA08-09 | 22,124,863 | ATGCGTCCACTCACGAAATGG | GCTAGTATATAGTTCGTACGCACG |
| PA08-12 | 22,598,723 | ACTCCACAAAAGGCAGTTGG | AATGGTCCAAGGTGTGCATT |
| PA08-16 | 23,264,842 | TGCCCATTTTTCAATTCTACG | ACTAAACCACCATGCCGTTG |
| ST69 | 23,590,777 | CGGAGAGAAAAGGACATGGA | GTTGGAGGAGCTCTAGAATTC |
| PA08-60 | 23,658,583 | AGGTGTGGTGGACCTACCTG | CCATTGCACAACCTTTTCCT |
| ST80 | 23,800,027 | ACTCCATCGCTTTAAGGCTG | CGTCAGAATTATGGAACTGAG |
| PA08-19 | 23,816,000 | GGTGTTGTAGGTTGCCGTTT | CTGGCAAGCTACTGTTTTAG |
| ST84 | 23,836,419 | CTTGGAGCTAATTCCTGTCTC | AAGGCTCATTCTGGGTCAAC |
| ST85 | 23,851,509 | TGAGCTGTTCTGCATCCTGT | TGTCTTAGCAGGTGTGCTTG |
| PA08-62 | 23,872,720 | TGGACCTAAATATCTGCAGCAC | GGCTAGTACATCTGCGTCACG |
| PA08-63 | 23,885,392 | AGCAACGACCATCATTTCGT | CTTTGTAATGTTGAATGGGAGG |
| ST97 | 23,897,269 | ATGTCAAGAAAATGAGTAGACG | CACACTCTGTTACCATTTTACAG |
| ST87 | 23,917,247 | ACGTACGGCAAAAGGCTGT | GACTTGGATACTACGGCAAG |
| ST89 | 23,952,818 | CAGGATGCATTCAGTAGCAG | CTGTGAAACACAAGCACAAGT |
| ST90 | 23,972,687 | CTACTATTGCTCCCACCATTC | CTCAGGCCTTATATGTGCATG |
| ST91 | 23,980,895 | TGATGCGTGTTTCATGACAAC | GGACCAGCCTAGAACAGCA |
| ST92 | 23,996,320 | TGCCAATATCTCTTGCCTCT | GGTGAACAACGACGCTCTAG |
| ST113 | 23,998,282 | ACTTAGCAAGCCCTTTCATATG | CAGCGAGGTGGTCTGGTCA |
| ST93 | 24,022,761 | GACAAATCTTCGTCGTGAGG | AGGTTTGGCATTGTGCCCAA |
| ST99 | 24,039,656 | ACGATACCATGTTTCTTCAGC | GTCAGGAGCTGGTAATGCCT |
| ST95 | 24,054,798 | GAACTGCAAGACCCTGCATC | CAGCGCTCTTTCAGATTTCG |
| PA08-20 | 24,072,874 | GATTGCATCTGCATCACTGC | CCACCTGACCAACCTGTTTT |
| ST103 | 24,090,925 | GTTAACTGAGCAATGAGGACT | CTTCGTTGCAAGGTCGGCTA |
| ST109 | 24,098,696 | CCAAACATCTGATTGGATTTGA | CTACTTTTCTCCGATACGGTC |
| ST104 | 24,117,957 | CTAGTGCAGAACAGAGGCTT | GAGTATCTCAGAACAATCTTGG |
| ST02 | 24,129,633 | GGTTCTCATTTCCTCGGTTC | GACACGATTTCATCAGTTCCA |
| ST107 | 24,146,093 | TCAAGATGCACCTGGTGTCT | CAAGCACAGTGCATATAGAGA |
| ST108 | 24,166,111 | CGGAGACGAAATCACGTCGA | GCCTCTGACTAGCAATCAGC |
| ST72 | 24,178,348 | GTCATGCAATTGTAGCTAAGC | GCTTAGCTTTCGCGACGACT |
| ST05 | 24,188,407 | CTCCATCAATCTCGAAGAATC | CATATGTATCCGCTGAACGA |
| RM256 | 24,273,349 | GACAGGGAGTGATTGAAGGC | GTTGATTTCGCCAAGGGC |
| ST54 | 24,274,701 | TGGGAAGAGGTGGTTTCGC | GCATTAGCATATCAAATGAACG |
| ST23 | 24,852,924 | CACAAGCTCGAATAAACTAGC | CGCACGATCGAGAGATCAG |

Finally, three recombinants were obtained from the IR68897B×NIL_107B-12 cross and one recombinant from the IR58025B×NIL_91B-42 cross, respectively. The fine-mapping result indicated that the genetic locus controlling stigma phenotype was located to about 142 kb region defined by ST97 and ST99 markers (FIG. 1B). The characteristics of allele dominance of qSTGL8.0 and the gene location within qSTGL8.0 locus governing long stigma phenotype were consistent between two OL alleles. This result suggests that the same gene sit on the qSTGL8.0 of each OL allele controls stigma phenotype.

### Example 3

### Knock-Out of OsEPFL1 Homologous Gene in the NIL-qSTGL8.0 Background Reverted to a Short Stigma Phenotype

In the fine-mapped 142 kb region, 20 genes were annotated in the rice reference genome database (FIG. 1C). Candidate genes were selected based on the protein functions, in silico gene expression analysis, and sequence comparisons to identify the corresponding gene(s) controlling the stigma size using transgenic approaches. Because of clear dominant inheritance patterns, the upstream genes like developmental process-related protein, transcription factors, and hormone-related protein were set as priority candidates rather than transposable elements-like genes and enzyme function proteins. Finally, three candidate genes Os08g37810 encoding transcription factor like protein, Os08g37840 encoding phosphate-induced protein 1, and Os08g37890 encoding rice epidermal patterning factor-like 1 (OsEPFL1) protein were selected. For gene validation tests, a CRISPR/Cas9 tool was applied to the NIL_6i14-191 possessing qSTGL8.0-OL in IR64 background for generation of knock-out (KO) of OL allele for each candidate gene, expecting reduced stigma length in the KO plants (see “Vector construction and rice transformation” in Material Method section). About 100 F₂ segregating embryos (IR64/IR64:IR64/OL:OL/OL=1:2:1 ratio) derived from the F₁s (IR64×NIL_6i14-191) were transformed with each CRISPR/Cas9 construct using Agrobacterium method. More than 120 To transgenic plants for each construct were obtained and were genotyped by using the ST109 marker (NIL_6i14-191 comprises an introgression including ST109 locus). Firstly, the homozygous (OL/OL) plants were selected for each construct and the CRISPR-Cas9 target region was sequenced by direct PCR products sequencing. More than seven KO plants were obtained for each candidate gene and stigma phenotyping was performed from more than 30 individual transgenic plants for each construct including all the KO plants. The KO plants for the Os08g37810 and Os08g37840 homologous genes did not alter stigma phenotype and all the phenotyped T₀ plants regardless of gene editing for the both genes showed long stigma phenotype, indicating that these two genes derived from the OL are not associated with stigma phenotype. However, all the KO plants for the Os08g37890 homologous gene exhibited short stigma phenotype compared to the control plants (FIGS. 2A-B). In addition, the long stigma phenotype was entirely dependent on the presence of the functional Os08g37890-OL allele from the CRISPR-Cas9 derived plants: Absence of the functional Os08g37890-OL allele by reading frame shift in a transgenic plants having IR64/OL or OL/OL background genotypes reverted to a short stigma phenotype, while the presence of the functional Os08g37890-OL allele because of no sequence change or in-frame deletion maintained a long stigma (Table 6).

TABLE 6
Correlation between the presence of the functional Os08g37890-OL allele and
long stigma phenotype from the CRISPR-Cas9 derived T₀ transgenic plants

| | | Sequence change | Functionality | |
|---|---|---|---|---|
| | Plant genotype^{a} | of Os08g37890 | of Os08g37890-OL | Stigma |
| Plant # | (Allele 1/Allele 2) | (Allele 1/Allele 2) | allele | phenotype |
| NIL_6i14-191_Con-P1 | OL/OL | | F/F | Long |
| IRS1493-001 | OL/OL | 1 bp ins/8 bp del | NF/NF | Short |
| IRS1493-008 | OL/OL | 1 bp del/1 bp ins | NF/NF | Short |
| IRS1493-009 | OL/OL | 4 bp del/1 bp ins | NF/NF | Short |
| IRS1493-010 | OL/OL | 4 bp del/8 bp del | NF/NF | Short |
| IRS1493-037 | OL/OL | 7 bp del/1 bp ins | NF/NF | Short |
| IRS1493-038 | OL/OL | 1 bp ins/3 bp del (inframe) | NF/F | Long |
| IRS1493-040 | OL/OL | 1 bp ins/1 bp ins | NF/NF | Short |
| IRS1493-041 | OL/OL | 7 bp del/1 bp ins | NF/NF | Short |
| IRS1493-042 | OL/OL | 1 bp ins/1 bp ins | NF/NF | Short |
| IRS1493-083 | OL/OL | 1 bp ins/1 bp ins | NF/NF | Short |
| IRS1493-115 | OL/OL | 4 bp del/1 bp ins | NF/NF | Short |
| IRS1493-116 | OL/OL | 4 bp del/1 bp ins | NF/NF | Short |
| IRS1493-118 | OL/OL | 5 bp del/1 bp ins | NF/NF | Short |
| IRS1493-119 | OL/OL | 4 bp del/1 bp ins | NF/NF | Short |
| IRS1493-120 | OL/OL | 1 bp ins/1 bp ins | NF/NF | Short |
| IRS1493-013 | IR64/OL | 1 bp ins/1 bp ins | —/NF | Short |
| IRS1493-017 | IR64/OL | 1 bp ins/1 bp ins | —/NF | Short |
| IRS1493-055 | IR64/OL | 67 bp del/3 bp del (inframe) | —/F | Long |
| IRS1493-063 | IR64/OL | 3 bp del (inframe)/NE | —/F | Long |
| IRS1493-099 | IR64/OL | 1 bp ins/2 bp del | —/NF | Short |
| IRS1493-033 | IR64/IR64 | 1 bp del/4 bp del | —/— | Short |
| IRS1493-062 | IR64/IR64 | 1 bp del/4 bp del | —/— | Short |
| IRS1493-086 | IR64/IR64 | 4 bp del/42 bp del | —/— | Short |
| IRS1493-121 | IR64/IR64 | NE/NE | —/— | Short |

^{a}Because immature F₂ embryos derived from the F₁s (IR64 × NIL_6i14-191) were used for rice transformation with pIRS1493 construct, the genotype of T₀ plants will be segregated as 1:2:1 (IR64/IR64:IR64/OL:OL/OL) ratio. Genotypes of each T₀ transgenic plant was identified by the ST109 marker and the CRISPR-Cas9 target region was sequenced. OL: O. longistaminata (IRGC 110404) allele, NE: not edited, NF: non-functional allele, F: functional allele

These results indicate that the Os08g37890 (OsEPFL1) homologous gene of the OL allele is responsible for long stigma phenotype in the NIL_6i14-191 possessing qSTGL8.0-OL (IRGC110404). In conclusion, the rice OsEPFL1 homologous gene of the OL located at qSTGL8.0 locus provided a long stigma phenotype and the gene was named as Oryza longistaminata long stigma 1 (OLLS1) in this study.

### Example 4

### Horizontal Transfer of OLLS1 to Indica Variety IR64 Drastically Increased Stigma Length

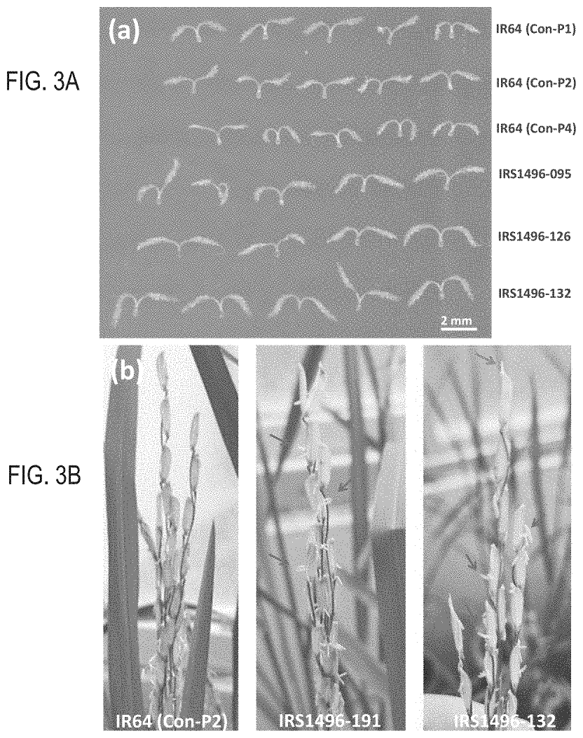

For further conformation, a complementation test was conducted using the OLLS1 allele from the OL (IRGC92664). The 4.4 kb genomic segment containing native promoter, CDS, and transcription terminator of OLLS1 was cloned from the NIL_107B-12 and it was transferred into an indica variety IR64 by using Agrobacterium method. Fifty T₀ transgenic plants and a tissue culture-derived control plants (IR64) were grown at a confined glasshouse. All the transgenic plants containing the 4.4 kb OLLS1 segment regardless of the T-DNA copy numbers showed drastically increased stigma length as well as high stigma exsertion rate (FIGS. 3A-B). This result concludes that the single dominant OLLS1 allele is responsible for a long-exerted stigma phenotype and is sufficient to make long-exserted stigma in short stigma rice varieties. Consequentially, both OLLS1-IRGC110404 and OLLS1-IRGC92664 genes which are located at the qSTGL8.0 (about 3 Mb) in the NIL_91B-42 and NIL_107B-12 respectively are corresponding genes for the long-exerted stigma phenotype and they have the same function in increasing stigma length.

### Example 5

### OLLS1 is Homolog to RAE2/GAD1 and OLLS1 is Strongly Expressed in Pistil

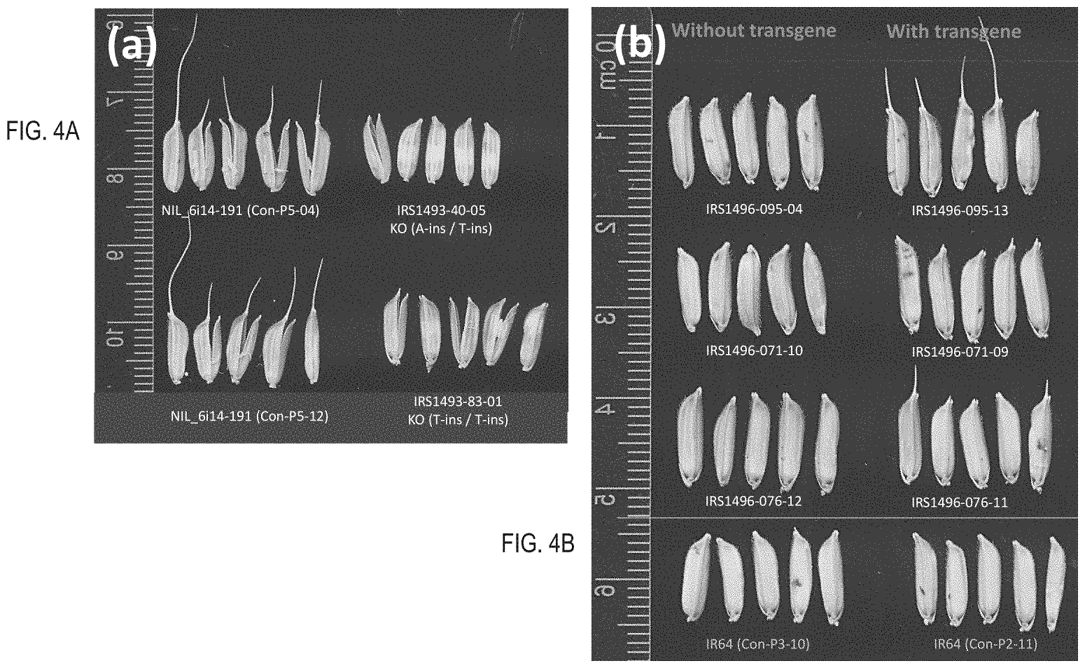

Os08g37890 encoding OsEPFL1 protein was previously identified as GAD1 (GRAIN NUMBER, GRAIN LENGTH AND AWN DEVELOPMENT1) which is originated from O. rufipogon and is associated with grain number per panicle, grain length, and awn development (Jin et al., 2016) and also known as RAE2 (REGULATOR OF AWN ELONGATION 2) which is from African cultivated rice species, O. glaberrima and is involved in awn development (Bessho-Uehara et al., 2016). The previously identified GAD1 and RAE2 alleles commonly control awn development. So awn phenotype was determined in both KO and complementation test transgenic plants. Awn phenotypes including awn presence/absence and awn length were variable at the levels of cropping seasons, plants in the same line, tillers in a plant, and spikelets on a panicle. However, awn in the NIL_6i14-191 disappeared when OLLS1 become null by CRISPR-Cas9 (FIG. 4A). In the complementation test lines, overall a short awn (less than 0.5 cm) was observed at the tip spikelet on each primary rachis from most of complementation transgenic plants while the background material IR64 has no awn (FIG. 3B). These results support that OLLS1 also has minor contribution in awn development. However, no significant genetic effect of OLLS1 on grain size was observed in the segregating T₁ plants derived from the To complementation test transgenic plants (FIG. 4B), suggesting that OLLS1 is not associated with grain size trait.

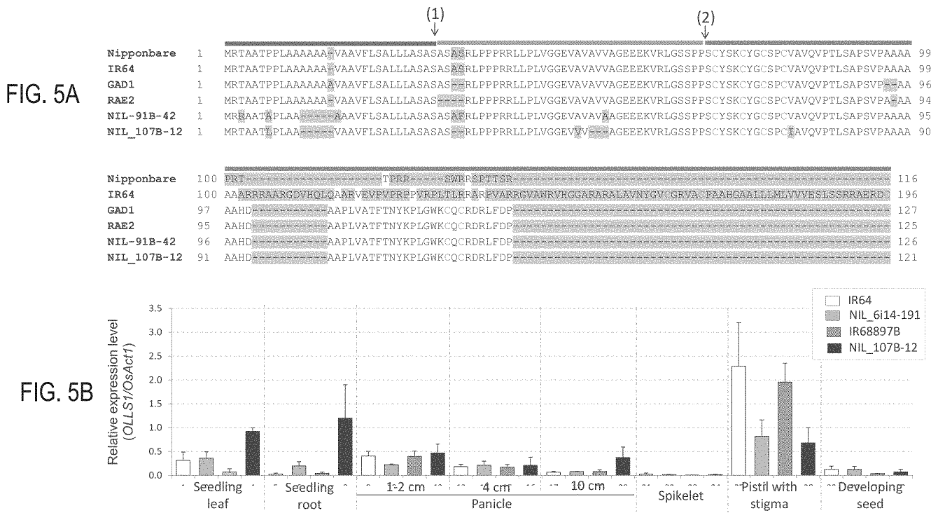

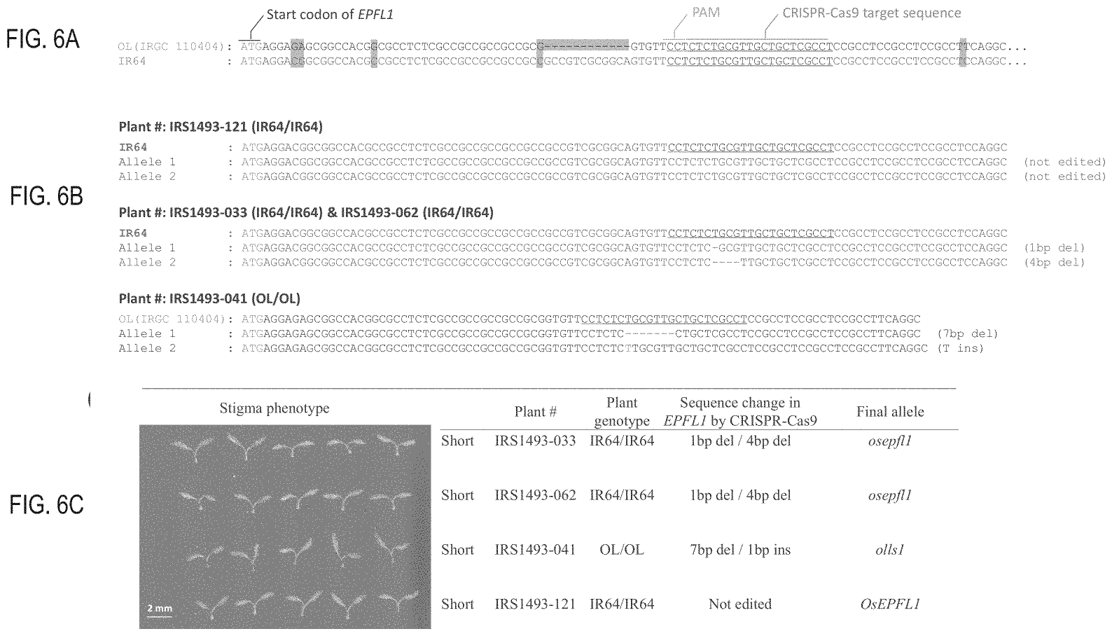

The protein coding sequences of OLLS1 from two different OL accessions were a bit different each other. The amino acid sequences among OLLS1 of two OL alleles, GAD1, RAE2, and OsEPFL1 of cultivated rice varieties. OLLS1, GAD1, and RAE2 comprise six conserved cysteine (C) residues which mediate proper formation of intramolecular disulfide bonds that are critical for peptide function, while the cultivated rice comprises putative non-functional EPFL1 protein consisting of 4 C in the reference Nipponbare and consisting of 7 C in IR64 (FIG. 5A). This result supports that both OLLS1 alleles encode functional EPFL1 protein like GAD1/RAE2 although they have several amino acid alterations. Through application of CRISPR-Cas9 tool to homozygous IR64 F₂s (IR64/IR64), KO of OsEPFL1-IR64 alleles was obtained (Table 6). There was no phenotypic difference between osepfl1-IR64 and the original OsEPFL1-IR64 alleles (FIG. 6), confirming that OsEPFL1 of IR64 is already non-functional allele.

To examine the spatial-temporal gene expression of OLLS1/OsEPFL1, qRT-PCR tests were performed from several tissues collected from the two NILs and their recurrent backgrounds. OLLS1/OsEPFL1 strongly expressed in pistil and mildly expressed in young panicles (FIG. 5B). This result supports that strong expression of OLLS1 alleles in female organ including stigma during spikelet development enlarged stigma size. However, expression of the nonfictional rice EPFL1 was higher than functional OLLS1 in pistil tissue. In root and leaf tissues, expression of OLLS1-IRGC92664 in the NIL_107B-12 was relatively higher than OLLS1-IRGC110404 in the NIL_91B-42. To compare the promoter sequences, we aligned the 4.4 kb genomic sequences of EPFL1 homologs from O. sativa (Nipponbare and IR64), O. glaberrima, O. rufipogon, O. nivara, O. barthii, O. glumaepatula, and O. longistaminata of AA genome Oryza species. The sequences of promoter regions of O. longistaminata are different to that of other species/accessions: two large InDel (>200 bp) and more than 20 SNPs are unique in the two OL accessions (FIG. 7). These OL specific nucleotide variations on the promoter region probably induced strong expression in stigma tissue, resulting in long stigma. In addition, three and two unique insertions (142 to 561 bp length) were found in the OL (IRGC92264) and OL (IRGC110404), respectively. This may be involved in expression difference between two OLs in seedling root and leaf.

### Example 6

### Transfer of OLLS1 Gene to the Commercial Hybrid Parental Lines, IR68897B/A by Precision Marker-Based Breeding Successfully Developed a Long-Exserted Stigma Lines

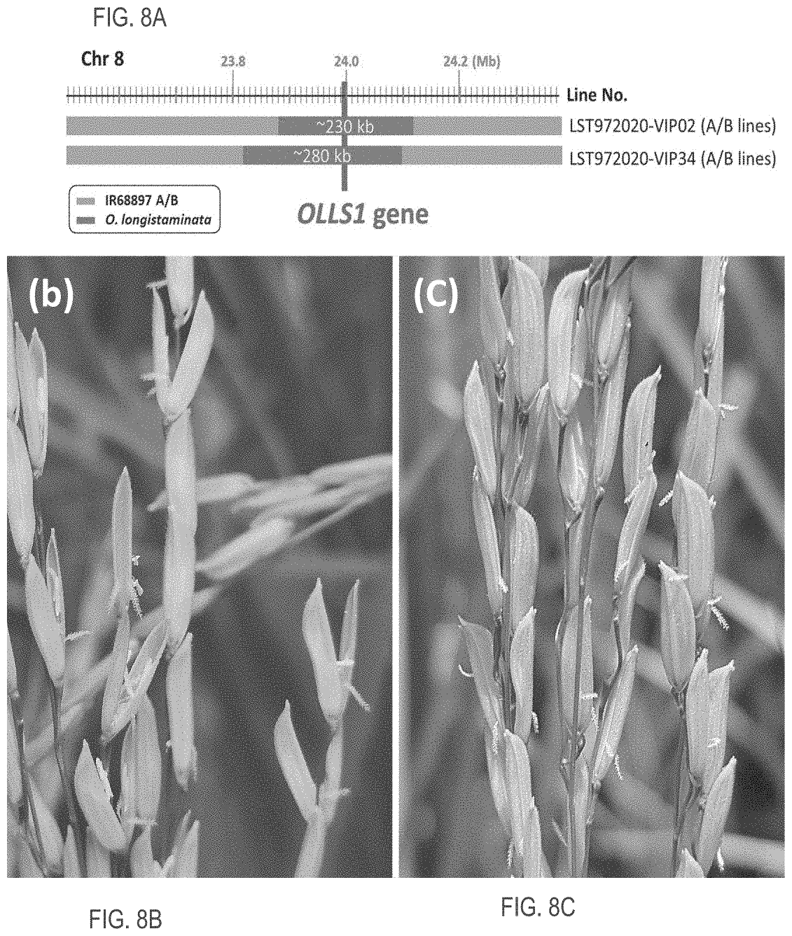

Precision marker-based breeding was conducted to increase stigma size and stigma exertion rate as well as to eliminate unexpected traits in the final breeding products which are caused by linkage drag and/or the gene-unlinked introgressions of the OLLS1 donor, O. longistaminata. The NIL_107B-12 possessing qSTGL8.0 (introgression size >3.0 Mb) was backcrossed with its recurrent parent IR68897B. To minimize linkage drag, the first round of recombinant selection (RS) was performed with 2,688 F2 plants with the OLLS1 flanking markers (Table 5), resulted in selection of eight F2 plants which had one side-trimmed introgression. The selected eight F₂ plants were backcrossed again. The backcrossed plants were genotyped by using ST89 marker and the heterozygous plants were selected and were self-pollinated for the second round of RS to trim another side. Through the 2^{nd} RS with ˜2,400 plants, finally three plants which had the smallest introgression of OLLS1 (230˜350 kb sizes) with different recombinant sites were selected (FIG. 8A) and these lines were experienced additional backcross (in total 3 times of backcross from the NIL_107B-12), resulted in elimination of the gene-unlinked donor introgressions. The final three lines in IR68897B background (LST972020-VIP02, LST972020-VIP34, LST972020-VIP12) were crossed with IR68897A line respectively to transfer the OLLS1 segment (230-350 kb) into A line. Finally the OLLS1 homozygous lines in B and A line backgrounds were obtained and the major agronomic traits were compared between the final breeding lines and their recurrent parental lines. As expected, stigma size and exsertion were drastically improved in the breeding lines possessing OLLS1 compared to the original parental lines (FIG. 8B-C). This result also supports that 230 kb-OLLS1 introgression within the qSTGL8.0 (˜3 Mb) is sufficient for causing a long-exserted stigma.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

Bae, S., Park, J. and Kim J.-S. (2014) Cas-OFFinder: A fast and versatile algorithm that searches for potential off-target sites of Cas9 RNA-guided endonucleases. Bioinformatics 30, 1473-1475.
Bessho-Uehara, K., Wang, D. R., Furuta, T., Minami, A., Nagai, K., Gamuyao, R., Asano K. et al. (2016) Loss of function at RAE2, a previously unidentified EPFL, is required for awnlessness in cultivated Asian rice. Proc. Natl Acad. Sci. USA, 113, 8969-8974.
Dubchak, I. and Ryaboy, D. V. (2006) VISTA family of computational tools for comparative analysis of DNA sequences and whole genomes. Methods Mol. Biol. 338, 69-89.
Jena, K. K., Marathi, B., Ramos, J., Diocton, IV. R., Vinarao, R., Prahalada, G. D. and Kim, S. R. (2016) Increasing hybrid seed production through higher outcrossing rate in cytoplasmic male sterile rice and related materials and methods. WO/2016/193953 (International Application No. PCT/IB2016/053294).
Jin, J., Hua, L., Zhu, Z., Tan, L., Zhao, X., Zhang, W., Liu, F. et al. (2016) GAD1 encodes a secreted peptide that regulates grain number, grain length, and awn development in rice domestication. Plant Cell, 28, 2453-2463.
Kim, S. R., Yang, J., An, G. and Jena, K. K. (2016) A simple DNA preparation method for high quality polymerase chain reaction in rice. Plant Breed. Biotechnol. 4, 99-106.
Slamet-Loedin, I. H., Chadha-Mohanty, P. and Torrizo, L. (2014) Agrobacterium-mediated transformation: rice transformation. Methods Mol. Biol. 1099, 261-271.

1. A method of producing a Gramineae plant, the method comprising:
(a) expressing in a Gramineae plant or plant cell a polynucleotide encoding OLLS1 as set forth in SEQ ID NO: 12 or 13 or a homolog thereof capable of increasing stigma length of the Gramineae plant, wherein when said expressing is by crossing the plant with another plant expressing said polypeptide, selecting for stigma length is performed using at least one marker located between ST87 to ST99; and
(b) growing or regenerating the plant.

2. A method of identifying a rice plant useful for crossing, the method comprising:
identifying in rice plants at least one marker located between ST87 to ST99 using marker assisted selection (MAS), wherein identification of said at least one marker is indicative of rice plant comprising a stigma length of interest.

3-7. (canceled)

8. The method of claim 1, wherein said marker is selected from the group consisting of ST97, ST87, ST89, ST90, ST91, ST92, ST113, ST93 and ST99.

9. The method of claim 1, wherein said marker is ST89.

10. The method of claim 8, wherein said marker is ST92 or ST113.

11. The method of claim 8, further comprising determining stigma length of the plant following said expressing.

12. A cultivated Gramineae plant being genetically modified to express a polypeptide encoding OLLS1 as set forth in SEQ ID NO: 12 or 13 or a homolog thereof capable of increasing stigma of the plant as compared to said stigma in a plant of same genetic background and developmental stage as the plant and not subjected to said genetic modification, wherein when said genetic modification is an introgression from Oryza longistaminata encoding said polypeptide, the length of the introgression is shorter than 350 or 300 Kb and comprising a marker selected from the group consisting of ST87, ST89, ST90, ST91, ST92, ST113, ST93 and ST99.

13-16. (canceled)

17. A cultivated rice plant comprising an introgression including at least one Oryza longistaminata quantitative trait locus (QTL) associated with stigma length positioned between markers ST87 to ST99 and said introgression being shorter than 350 or 300 Kb.

18. (canceled)

19. The plant of claim 17, wherein said introgression is shorter than 100 Kb.

20-22. (canceled)

23. The method of claim 1, wherein the plant is male sterile.

24. The method of claim 1, wherein the plant is environment-sensitive genic male sterile.

25. The method of claim 1, wherein the plant is a cytoplasmic male sterile line.

26. The method of claim 1, wherein the plant is a maintainer line.

27. The method of claim 1, wherein the plant has an out-crossing rate of at least 60%.

28. A cultivated hybrid Gramineae plant having the plant of claim 12 as a parent or an ancestor.

29-35. (canceled)

36. A method of producing a cytoplasmic male sterile Gremineae plant comprising a long stigma trait of Oryza longistaminata, the method comprising crossing the plant of a stable cytoplasmic male sterile line of claim 25 with a rice plant of a suitable maintainer line.

37. A method for increasing hybrid seed set in a Gramineae plant comprising:
providing a male sterile Gramineae plant comprising a long stigma trait of Oryza longistaminata according to claim 12; and
pollinating the cytoplasmic male sterile plant comprising a long stigma trait of Oryza longistaminata with pollen of a suitable Gramineae line.

38. The method of claim 37, wherein said male sterile Gramineae plant is environment-sensitive genic male sterile.

39. The method of claim 37, wherein said male sterile Gramineae plant is cytoplasmic genetic male sterile and said suitable Gramineae line is a restorer line.

40. A method for producing hybrid rice seed comprising:
carrying out the method of claim 37; and
collecting hybrid seed set on the cytoplasmic male sterile plant comprising the long stigma trait of Oryza longistaminata.

41. A method of producing meal, the method comprising:
(a) growing and collecting seeds of the hybrid plant of claim 28; and
(b) processing said seeds to meal.

42. The method of claim 2, wherein the Gramineae plant is selected from the group consisting of cultivated rice, wheat and maize.
